(19) United States
(12) Patent Application Publication
(10) Pub. No.: US 2025/0277034 A1
(43) Pub. Date: Sep. 4, 2025

(54) Caninized Antibodies

(71) Applicant: INTERVET INC., RAHWAY, NJ (US)

(72) Inventors: Mohamad Morsey, Omaha, NE (US); Yuanzheng Zhang, Somerset, NJ (US); Ian Tarpey, St. Ives (GB)

(73) Assignee: INTERVET INC., RAHWAY, NJ (US)

(21) Appl. No.: 19/051,538

(22) Filed: Feb. 12, 2025

Related U.S. Application Data
(63) Continuation of application No. 17/573,870, filed on Jan. 12, 2022, now Pat. No. 12,252,536.
Application No. 17/573,870 is a division of application No. 16/130,578, filed on Sep. 13, 2018, now Pat. No. 11,248,047.
Application No. 16/130,578 is a division of application No. 15/105,211, filed on Jun. 16, 2016, now Pat. No. 10,106,607.
(60) Provisional application No. 62/030,812, filed on Jul. 30, 2014.
(60) Provisional application No. 61/918,847, filed on Dec. 20, 2013.
(60) Provisional application No. 61/918,946, filed on Dec. 20, 2013.

(51) Int. Cl. C07K 16/28; A61K 39/00; A61K 39/395; A61P 37/04

(52) U.S. Cl. CPC C07K 16/2803; A61K 39/3955; A61P 37/04; C07K 16/2818; C07K 16/2896; A61K 39/00; A61K 2039/55516; C07K 2317/24; C07K 2317/34; C07K 2317/52; C07K 2317/565; C07K 2317/76; C07K 2317/92

(57) ABSTRACT

The present invention discloses caninized antibodies with specific properties. The present invention also discloses caninized murine antibodies against canine PD-1 that have a high binding affinity for canine PD-1. The invention further discloses the use of the caninized antibodies of the present invention in the treatment of cancer in dogs.

### CROSS-REFERENCE TO RELATED APPLICATIONS

This application is a continuation of U.S. patent application Ser. No. 17/573,870, filed Jan. 12, 2022, now allowed, which is a division of U.S. patent application Ser. No. 16/130,578, filed Sep. 13, 2018, now U.S. Pat. No. 11,248,047, which is a division of U.S. patent application Ser. No. 15/105,211 filed Jun. 16, 2016, now U.S. Pat. No. 10,106,607, which is a National Stage Entry Under 371 of PCT/EP2014/078653, filed Dec. 19, 2014, which claims benefit of U.S. Provisional Application Ser. No. 62/030,812, filed on Jul. 30, 2014, U.S. Provisional Application Ser. No. 61/918,847, filed on Dec. 20, 2013, and U.S. Provisional Application Ser. No. 61/918,946, filed on Dec. 20, 2013, the contents of all of which are hereby incoporated by reference in their entireties.

### REFERENCE TO SEQUENCE LISTING SUBMITTED ELECTRONICALLY

The contents of the electronic sequence listing (23799USCNT_SL.xml; Size: 308,735 bytes; and Date of Creation: Jan. 31, 2025) are herein incorporated by reference in its entirety.

### FIELD OF THE INVENTION

The present invention relates to caninized antibodies with specific properties. The present invention also relates to caninized antibodies against canine PD-1 that have specific sequences and a high binding affinity for canine PD-1. The invention further relates to use of the antibodies of the present invention in the treatment of dogs, including cancer treatment.

### BACKGROUND OF THE INVENTION

Canine antibodies (also referred to as immunoglobulin G or IgG) are large tetrameric proteins of about 150 Kd. Each IgG protein is composed of two identical light chains of about 25 Kd each, and two identical heavy chains of about 50 Kd each. There are four known IgG heavy chain subclasses of canine IgG and they are referred to as IgGA, IgGB, IgGC, and IgGD. There are two types of light chains; kappa and lambda chains. Each of the kappa or lambda light chains is composed of one variable domain (VL) and one constant domain (CL). Each of the two heavy chains consists of one variable domain (VH) and three constant domains referred to as CH-1, CH-2, and CH-3. The CH-1 domain is connected to the CH-2 domain via an amino acid sequence referred to as the “hinge” or alternatively as the “hinge region”. In humans, IgG exists in one of four subclasses referred to as IgG1, IgG2, IgG3, and IgG4. The subclass of IgG is determined largely by the sequence of the hinge region, which differs among the four subclasses of IgG. The two heavy chains are linked to each other by disulfide bonds and each heavy chain is linked to one of the light chains also through a disulfide bond.

Digestion of IgG antibodies with the enzyme papain breaks the antibody molecule in the hinge region and results in the formation of three fragments. Two of these fragments are identical and each consists of the light chain held together with the VH and CH1 domains of the heavy chain. These fragments are called the “Fab” fragments and they contain the antigen binding sites of the antibody. The third fragment that results from digestion with papain is called the “Fc” and it contains the remainder of the two heavy chains held together by disulfide bonds. The Fc thus contains a dimer consisting of the CH2 and CH3 domain of each of the two heavy chains. While the Fab enables the antibody to bind to its cognate epitope, the Fc enables the antibody to mediate immune effector functions such as antibody dependent cellular cytotoxicity (ADCC), antibody-dependent phagocytosis (ADCP) and complement dependent cytotoxicity (CDC).

It is well known in the art that IgG antibodies mediate effector functions such as ADCC and ADCP through binding of their Fc portion to a family of proteins known as Fc_{γ} receptors, whereas CDC is mediated through the binding of the Fc to the first component of complement, C1q. It is also well known in the art that different IgG sub-classes differ in their capacity to mediate these effector functions. For example, human IgG1 displays strong ADCC and CDC, whereas IgG4 displays a weak to no ADCC and CDC. In addition, methods for identification of which IgG sub-classes display or lack effector functions are well known in the art.

Approaches that rely on use of monoclonal antibodies for therapeutic purposes require the design of fit-for-purpose antibodies or antibody fragments to achieve the desired therapeutic response. For example, some therapeutic approaches for cancer require the therapeutic antibodies to have enhanced effector functions, while others require the effector functions to be significantly reduced or eliminated altogether. Enhancement or elimination of effector functions may be achieved through introduction of one or more amino acid mutations (substitutions) in the Fc portion of the antibody so as to enhance or reduce binding to FC_{γ} receptors and the first component of complement. There are numerous reports in the prior art describing amino acid substitutions that may be introduced into an antibody molecule in order to modulate its effector functions. For example, Shields et al., [J. of Biol. Chem., 276 (9): 6591-6604 (2001)] disclosed that an asparagine to alanine (N297A) substitution, which result in a non-glycosylated antibody, significantly reduced antibody binding to several Fc_{γ} receptors. Additionally, Shields et al., disclosed that an aspartic acid-to-alanine (D265A) substitution also significantly reduced binding of the antibody to Fc, receptors. Each of the N297A and D265A substitutions were also shown to significantly impair CDC. There are other similar reports identifying potential substitutions to reduce or eliminate effector function in antibodies [e.g., Sazinsky et al., Proc. Nat. Acad. Sci., 105:20167-20172 (2008), Alegre et al., Transplantation, 57:1537-1543 (1994), Hutchins et al., Proc. Nat. Acad. Sci. 92:11980-11984 (1994), McEarchem et al., Blood, 109:1185-1192 (2007)].

An immunoinhibitory receptor that is primarily expressed on activated T and B cells, Programmed Cell Death Receptor 1, also referred to as Programmed Death Receptor 1 (PD-1), is a member of the immunoglobulin superfamily related to CD28 and CTLA-4. PD-1 and like family members are type I transmembrane glycoproteins containing an extracellular Ig Variable-type (V-type) domain that binds its ligands and a cytoplasmic tail that binds signaling molecules. The cytoplasmic tail of PD-1 contains two tyrosine-based signaling motifs, an ITIM (immunoreceptor tyrosine-based inhibition motif) and an ITSM (immunoreceptor tyrosine-based switch motif).

PD-1 attenuates T-cell responses when bound to Programmed Cell Death Ligand 1, also referred to as Programmed Death Ligand 1 (PD-L1), and/or Programmed Cell Death Ligand 2, also referred to as Programmed Death Ligand 2 (PD-L2). The binding of either of these ligands to PD-1 negatively regulates antigen receptor signaling. Blocking the binding of PD-L1 to PD-1 enhances tumor-specific CD8⁺ T-cell immunity, while aiding the clearance of tumor cells by the immune system. The three-dimensional structure of murine PD-1, as well as the co-crystal structure of mouse PD-1 with human PD-L1 have been reported [Zhang et al., Immunity 20:337-347 (2004); Lin et al., Proc. Natl. Acad. Sci. USA 105:3011-3016 (2008)].

PD-L1 and PD-L2 are type I transmembrane ligands that contain both IgV- and IgC-like domains in the extracellular region along with short cytoplasmic regions with no known signaling motifs. Both PD-L1 and PD-L2 are either constitutively expressed or can be induced in a variety of cell types, including non-hematopoietic tissues as well as various tumor types. PD-L1 is not only expressed on B, T, myeloid and dendritic cells (DCs), but also on peripheral cells, such as microvascular endothelial cells and non-lymphoid organs e.g., heart or lung. In contrast, PD-L2 is only found on macrophages and DCs. The expression pattern of PD-1 ligands suggests that PD-1 plays a role in maintaining peripheral tolerance and may further serve to regulate self-reactive T- and B-cell responses in the periphery.

In any case, it is now abundantly clear that PD-1 plays a critical role in at least certain human cancers, presumably by mediating immune evasion. Accordingly, PD-L1 has been shown to be expressed on a number of mouse and human tumors and is inducible by IFN-γ in the majority of PD-L1 negative tumor cell lines [Iwai et al., Proc. Natl. Acad. Sci. U.S.A. 99:12293-12297 (2002); Strome et al., Cancer Res., 63:6501-6505 (2003)]. Furthermore, the expression of PD-1 on tumor infiltrating lymphocytes and/or PD-L1 on tumor cells has been identified in a number of primary human tumor biopsies. Such tumor tissues include cancers of the lung, liver, ovary, cervix, skin, colon, glioma, bladder, breast, kidney, esophagus, stomach, oral squamous cell, urothelial cell, and pancreas, as well as tumors of the head and neck [Brown et al., J. Immunol. 170:1257-1266 (2003); Dong et al., Nat. Med. 8:793-800 (2002); Wintterle et al., Cancer Res. 63:7462-7467 (2003); Strome et al., Cancer Res., 63:6501-6505 (2003); Thompson et al., Cancer Res. 66:3381-5 (2006); Thompson et al., Clin. Cancer Res. 13:1757-1761 (2007); Nomi et al., Clin. Cancer Res. 13:2151-2157. (2007)]. More strikingly, PD-ligand expression on tumor cells has been correlated to poor prognosis of human cancer patients across multiple tumor types [reviewed in Okazaki and Honjo, Int. Immunol. 19:813-824 (2007)].

Moreover, Nomi et al. [Clin. Cancer Res. 13:2151-2157 (2007)] demonstrated the therapeutic efficacy of blocking the binding of PD-L1 to PD-1 in a murine model of aggressive pancreatic cancer through administering either PD-1 or PD-L1 directed antibody. These antibodies effectively promoted tumor reactive CD8⁺ T cell infiltration into the tumor resulting in the up-regulation of anti-tumor effectors including IFN-γ, granzyme B, and perforin. Similarly, the use of antibodies to block the binding of PD-L1 and PD-1 significantly inhibited tumor growth in a model of mouse squamous cell carcinoma [Tsushima et al., Oral Oncol. 42:268-274 (2006)].

In other studies, transfection of a murine mastocytoma line with PD-L1 led to decreased lysis of the tumor cells when co-cultured with a tumor-specific CTL clone. Lysis was restored when anti-PD-L1 monoclonal antibody was added 12293-12297 (2002). In vivo, blocking the PD1/PD-L1 interaction was shown to increase the efficacy of adoptive T cell transfer therapy in a mouse tumor model Strome et al., Cancer Res., 63:6501-6505 (2003). Further evidence for the role of PD-1 in cancer treatment comes from experiments performed with PD-1 knockout mice in which PD-L1 expressing myeloma cells grew only in wild-type animals (resulting in tumor growth and associated animal death), but not in PD-1 deficient mice [Iwai Y. et al., Proc. Natl. Acad. Sci. U.S.A. 99:12293-12297 (2002)]. More recently, antibodies against PD-1 (including humanized murine monoclonal antibodies against human PD-1) have shown at least initial success in cancer therapy in humans [see e.g., U.S. Pat. No. 8,354,509 B2, U.S. Pat. No. 8,008,449 B2, and U.S. Pat. No. 7,595,048 B2].

Anti-PD-1 antibodies may also be useful in chronic viral infection. Memory CD8⁺ T cells generated after an acute viral infection are highly functional and constitute an important component of protective immunity. In contrast, chronic infections are often characterized by varying degrees of functional impairment (exhaustion) of virus-specific T-cell responses, and this defect is a principal reason for the inability of the host to eliminate the persisting pathogen. Although functional effector T cells are initially generated during the early stages of infection, they gradually lose function during the course of a chronic infection. Barber et al. [Nature 439:682-687 (2006)] showed that mice infected with a laboratory strain of LCMV developed chronic infection resulted in high levels of virus in the blood and other tissues. These mice initially developed a robust T cell response, but eventually succumbed to the infection upon T cell exhaustion. Barber et al. found that the decline in number and function of the effector T cells in chronically infected mice could be reversed by injecting an antibody that blocked the interaction between PD-1 and PD-L1.

The citation of any reference herein should not be construed as an admission that such reference is available as “prior art” to the instant application.

### SUMMARY OF THE INVENTION

The present invention provides a canine fragment crystallizable region (cFc region) of an antibody in which the cFc has been genetically modified to augment, decrease, or eliminate one or more effector functions. In one aspect of the present invention, the genetically modified cFc decreases or eliminates one or more effector functions. In another aspect of the invention the genetically modified cFc augments one or more effector function.

In certain embodiments, the genetically modified cFc region is a genetically modified canine IgGB Fc region. In another such embodiment, the genetically modified cFc region is a genetically modified canine IgGC Fc region. In a particular embodiment the effector function is antibody-dependent cytotoxicity (ADCC) that is augmented, decreased, or eliminated. In another embodiment the effector function is complement-dependent cytotoxicity (CDC) that is augmented, decreased, or eliminated. In yet another embodiment, the cFc region has been genetically modified to augment, decrease, or eliminate both the ADCC and the CDC.

The present invention further provides canine frames and/or full length heavy chains that comprise the genetically modified cFc regions. Accordingly, the present invention provides full length heavy chains of antibodies in which the full length heavy chains comprise the genetically modified cFc regions of the present invention. Such full length heavy chains can also be combined with corresponding canine light (kappa or lambda) chains to form a complete antibody. In particular embodiments of this type, the resulting antibody binds to a particular canine antigen with specificity. In certain such embodiments the canine antigen is canine PD-1. In yet other embodiments the canine antigen is canine PD-L1. In still other embodiments, the canine antigen is the IL-4 recepter alpha chain. In yet other embodiments the canine antigen is canine thymic stromal lymphopoietin protein (cTSLP) [see, U.S. Pat. No. 7,718,772 B2, the contents of which are hereby incorporated by reference in their entireties.]

In certain embodiments, the genetically modified cFc region comprises the amino acid sequence of SEQ ID NO: 130 (or SEQ ID NO: 132) in which one to seven of the following amino acid residues are replaced by another amino acid residue at the indicated positions: P4, D31, N63, G64, T65, A93, or P95. The amino acid substituting for P4, D31, N63, G64, T65, A93, and/or P95 are individually selected from one of the other 19 standard naturally occurring amino acids, as listed in Table 1 below. The present invention further provides variants of the genetically modified cFc regions that comprise an amino acid sequence that is 90%, 95%, 98%, or 99% identical to the amino acid sequence of such genetically modified cFc regions and retain at least 50%, 75%, 90%, 95%, or more of the augmentation, decrease, or elimination of the ADCC and/or the CDC as the genetically modified cFc regions comprising the amino acid sequence of SEQ ID NO: 130 (or SEQ ID NO: 132) in which one or more of the following amino acid residues were replaced: i.e., at P4, D31, N63, G64, T65, A93, or P95.

In other embodiments two to five of the following amino acid residues are replaced by another amino acid residue at the indicated positions: P4, D31, N63, G64, T65, A93, or P95. In particular embodiments of this type, the genetically modified cFc region comprises the amino acid sequence of SEQ ID NO: 130 or SEQ ID NO: 132 with the following substitutions: P4A, D31A, N63A, A93G, and P95A. In related embodiments, the genetically modified cFc region comprises the amino acid sequence of SEQ ID NO: 130 or SEQ ID NO: 132 with the following substitutions: P4A, D31A, N63A, and P95A. In other embodiments, the genetically modified cFc region comprises the amino acid sequence of SEQ ID NO: 130 or SEQ ID NO: 132 with substitutions at D31 and N63. In particular embodiments of this type, the aspartic acid residue at position 31 is replaced with a glutamic acid residue, an asparagine residue, or an alanine residue, whereas the asparagine residue at position 63 is replaced with a glutamine residue, a histidine residue, or an alanine residue. In a more particular embodiment of this type, the genetically modified cFc region comprises the amino acid sequence of SEQ ID NO: 130 or SEQ ID NO: 132 with the following substitutions: D31A and N63A. In particular embodiments, the genetically modified cFc region is encoded by the nucleotide sequence of SEQ ID NO: 129 or SEQ ID NO: 131 comprising nucleotide changes that correspond to the amino acid sequences that they encode.

In another embodiments, the genetically modified cFc region comprises the amino acid sequence of SEQ ID NO: 130 or SEQ ID NO: 132 with the substitution at A93. In a particular embodiment of this type, the substitution is A93G. In a related embodiment the the substitution is A93S. As shown below in Example 4, the substitution of A93G leads to an enhancement in complement C1q binding, which is indicative of increasing CDC activity.

In related embodiments the genetically modified cFc region further comprises a hinge region that comprises the amino acid sequence of SEQ ID NO: 109. In other embodiments the genetically modified Fc region further comprises a hinge region that comprises the amino acid sequence of SEQ ID NO: 110. In still other embodiments the genetically modified Fc region further comprises a hinge region that comprises the amino acid sequence of SEQ ID NO: 111. In yet other embodiments the genetically modified Fc region further comprises a genetically modified hinge region that comprises the amino acid sequence of SEQ ID NO: 112.

In alternative embodiments, the present invention provides a canine IgGD Fc region with a genetically modified hinge region from a canine IgGD antibody, a hinge region from a canine IgGA antibody, a hinge region from a canine IgGB antibody, or a hinge region from a canine IgGC antibody. Moreover, the present invention provides full length heavy chains of antibodies in which the full length heavy chains comprise the canine IgGD Fc region of the present invention with a genetically modified hinge region from a canine IgGD antibody, a hinge region from a canine IgGA antibody, a hinge region from a canine IgGB antibody, or a hinge region from a canine IgGC antibody. Such full length heavy chains also can be combined with corresponding canine light (kappa or lambda) chains to form a complete antibody.

Accordingly, the present invention provides a canine IgGD Fc region that further comprises a genetically modified hinge region from a canine IgGD antibody. In particular embodiments of this type the canine IgGD Fc region and genetically modified hinge region comprise the amino acid sequence of SEQ ID NO: 6 or an amino acid sequence that is 90%, 95%, 98%, or 99% identical to the amino acid sequence of SEQ ID NO: 6, which comprises a proline residue at position 10 (P10). In a more particular embodiment the canine IgGD Fc region and genetically modified hinge region is encoded by the nucleotide sequence of SEQ ID NO: 5. In other embodiments, the canine IgGD Fc region further comprises a hinge region from a canine IgGA antibody. In particular embodiments of this type the canine IgGD Fc region and hinge region comprise the amino acid sequence of SEQ ID NO: 8 or an amino acid sequence that is 90%, 95%, 98%, or 99% identical to the amino acid sequence of SEQ ID NO: 8. In a more particular embodiment the canine IgGD Fc region and hinge region is encoded by the nucleotide sequence of SEQ ID NO: 7. In still other embodiments, the canine IgGD Fc region further comprises a hinge region from a canine IgGB antibody. In particular embodiments of this type the canine IgGD Fc region and hinge region comprise the amino acid sequence of SEQ ID NO: 10 or an amino acid sequence that is 90%, 95%, 98%, or 99% identical to the amino acid sequence of SEQ ID NO: 10. In a more particular embodiment the canine IgGD Fc region and hinge region is encoded by the nucleotide sequence of SEQ ID NO: 9. In yet other embodiments, the canine IgGD Fc region further comprises a hinge region from a canine IgGC antibody. In particular embodiments of this type the canine IgGD cFc region and hinge region comprise the amino acid sequence of SEQ ID NO: 12 or an amino acid sequence that is 90%, 95%, 98%, or 99% identical to the amino acid sequence of SEQ ID NO: 12. In a more particular embodiment the canine IgGD cFc region and hinge region is encoded by the nucleotide sequence of SEQ ID NO: 11. The present invention further provides caninized antibodies that comprise these canine IgGD Fc regions and hinge regions. In a particular embodiment the caninized antibody or antigen binding fragment thereof binds canine Programmed Death Receptor 1 (canine PD-1) with specificity.

The present invention therefore provides caninized anti-canine PD-1 antibodies with specificity and/or that have a high binding affinity for canine PD-1. In particular embodiments, the caninized anti-canine PD-1 antibodies also have the ability to block the binding of canine PD-1 to canine PD-L1. In specific embodiments the caninized anti-canine PD-1 antibodies have a high binding affinity to canine PD-1, as well as have the ability to also block the binding of canine PD-1 to canine PD-L2. The caninized antibodies or antigen binding fragments thereof that specifically bind canine PD-1 can comprise a canine IgG heavy chain of the present invention and a canine kappa or lambda light chain. In particular embodiments the caninized anti-canine PD-1 antibodies are caninized murine anti-canine PD-1 antibodies. The present invention also relates to use of such caninized antibodies in the treatment of disease such as cancer and/or those due to infections.

In particular embodiments the caninized anti-canine PD-1 antibody comprises a genetically modified cFc region of the present invention. In alternative embodiments the caninized anti-canine PD-1 antibody comprises the canine IgGD Fc region with a genetically modified hinge region from a canine IgGD antibody, a hinge region from a canine IgGA antibody, a hinge region from a canine IgGB antibody, or a hinge region from a canine IgGC antibody. The present invention further provides such caninized anti-canine PD-1 antibodies comprising the canine frames of the present invention in combination with CDRs obtained from mouse anti-canine PD-1 antibodies, i.e., three light chain CDRs: CDR light 1 (CDRL1), CDR light 2 (CDRL2), and CDR light 3 (CDRL3) and three heavy chain CDRs CDR heavy 1 (CDRH1), CDR heavy 2 (CDRH2) and CDR heavy 3 (CDRH3).

In particular embodiments, the caninized murine anti-canine PD-1 antibodies comprise the genetically modified cFc region of IgGB or IgGC of the present invention or alternatively, the canine IgGD Fc region, together with a genetically modified hinge region from a canine IgGD antibody, a hinge region from a canine IgGA antibody, a hinge region from a canine IgGB antibody, or a hinge region from a canine IgGC antibody in combination with CDRs obtained from mouse anti-canine PD-1 antibodies. Moreover, the present invention not only provides caninized mouse anti-canine PD-1 antibodies with specific CDRs as detailed herein, but further provides caninized mouse anti-canine PD-1 antibodies comprising conservatively modified variants of those CDRs as well as variants that comprise (e.g., share) the same canonical structure.

Accordingly in particular embodiments the caninized anti-canine PD-1 antibody further comprises complementary determining regions (CDRs) in which the CDRs have canonical structures of: H1-1, H2-1, and H3-6, respectively for CDR1, CDR2, and CDR3 of the heavy chain, i.e., CDR1 of the heavy chain has the canonical structure class 1, CDR2 of the heavy chain has the canonical structure class 1, and CDR3 of the heavy chain has the canonical structure class 6. In even more particular embodiments, the CDRs for the corresponding light chains have canonical structures of: L1-3, L2-1, and L3-1, respectively for CDR1, CDR2, and CDR3 of the light chain. In other embodiments the caninized anti-canine PD-1 antibody further comprises complementary determining regions (CDRs) in which the CDRs have canonical structures of: H1-1, H2-1, and H3-11, respectively for CDR1, CDR2, and CDR3 of the heavy chain. In even more particular embodiments of this type, the CDRs for the corresponding light chains have canonical structures of: L1-2A, L2-1, and L3-1, respectively for CDR1, CDR2, and CDR3 of the light chain. In still other embodiments the caninized anti-canine PD-1 antibody further comprises complementary determining regions (CDRs) in which the CDRs have canonical structures of: H1-1, H2-2A, and H3-11, respectively for CDR1, CDR2, and CDR3 of the heavy chain. In even more particular embodiments of this type, the CDRs for the corresponding light chains have canonical structures of: L1-2A, L2-1, and L3-1, respectively for CDR1, CDR2, and CDR3 of the light chain. In yet other embodiments the caninized anti-canine PD-1 antibody further comprises complementary determining regions (CDRs) in which the CDRs have canonical structures of: H1-1, H2-2A, and H3-13, respectively for CDR1, CDR2, and CDR3 of the heavy chain. In even more particular embodiments of this type, the CDRs for the corresponding light chains have canonical structures of: L1-4, L2-1, and L3-1, respectively for CDR1, CDR2, and CDR3 of the light chain.

In more particular embodiments, the caninized antibody of the present invention or antigen binding fragment thereof comprises one or more of the heavy chain complementary determining region 1 (VH CDR1) with an amino acid sequence of SEQ ID NO: 27, SEQ ID NO: 28, SEQ ID NO: 29, or SEQ ID NO: 30. In another embodiment, the heavy chain complementary determining region 2 (VH CDR2) comprises an amino acid sequence of SEQ ID NO: 31, SEQ ID NO: 32, SEQ ID NO: 33, SEQ ID NO: 34, or SEQ ID NO: 35. In still another embodiment the heavy chain complementary determining region 3 (VH CDR3) comprises an amino acid sequence of SEQ ID NO: 36, SEQ ID NO: 37, SEQ ID NO: 38, or SEQ ID NO: 146. In a particular embodiment of this type, the caninized antibody or antigen binding fragment comprises both a VH CDR1 comprising an amino acid sequence of SEQ ID NO: 27, SEQ ID NO: 28, SEQ ID NO: 29, or SEQ ID NO: 30 and a VH CDR2 comprising an amino acid sequence of SEQ ID NO: 31, SEQ ID NO: 32, SEQ ID NO: 33, SEQ ID NO: 34, or SEQ ID NO: 35. In another such embodiment, the caninized antibody or antigen binding fragment comprises both a VH CDR1 comprising an amino acid sequence of SEQ ID NO: 27, SEQ ID NO: 28, SEQ ID NO: 29, or SEQ ID NO: 30 and a VH CDR3 comprising an amino acid sequence of SEQ ID NO: 36, SEQ ID NO: 37, SEQ ID NO: 38, or SEQ ID NO: 146. In yet another such embodiment, the caninized antibody or antigen binding fragment comprises both a VH CDR2 comprising an amino acid sequence of SEQ ID NO: 31, SEQ ID NO: 32, SEQ ID NO: 33, SEQ ID NO: 34, or SEQ ID NO: 35 and a VH CDR3 comprising an amino acid sequence of SEQ ID NO: 36, SEQ ID NO: 37, SEQ ID NO: 38, or SEQ ID NO: 146. In still another such embodiment, the caninized antibody or antigen binding fragment comprises a VH CDR1 comprising an amino acid sequence of SEQ ID NO: 27, SEQ ID NO: 28, SEQ ID NO: 29, or SEQ ID NO: 30, a VH CDR2 comprising an amino acid sequence of SEQ ID NO: 31, SEQ ID NO: 32, SEQ ID NO: 33, SEQ ID NO: 34, or SEQ ID NO: 35, and a VH CDR3 comprising an amino acid sequence of SEQ ID NO: 36, SEQ ID NO: 37, SEQ ID NO: 38, or SEQ ID NO: 146.

In particular embodiments, the caninized antibody or antigen binding fragment also comprises a light chain complementary determining region 1 (VL CDR1) comprising an amino acid sequence of SEQ ID NO: 13, SEQ ID NO: 14, or SEQ ID NO: 15. In related embodiments the light chain complementary determining region 2 (VL CDR2) comprises an amino acid sequence of SEQ ID NO: 16, SEQ ID NO: 17, SEQ ID NO: 18, SEQ ID NO: 19, SEQ ID NO: 20, or SEQ ID NO: 21. In still another embodiment the light chain complementary determining region 3 (VL CDR3) comprises an amino acid sequence of SEQ ID NO: 22, SEQ ID NO: 23, SEQ ID NO: 24, SEQ ID NO: 25, or SEQ ID NO: 26. In a particular embodiment of this type, the caninized antibody or antigen binding fragment comprises both a VL CDR1 comprising an amino acid sequence of SEQ ID NO: 13, SEQ ID NO: 14, or SEQ ID NO: 15 and a VL CDR2 comprising an amino acid sequence of SEQ ID NO: 16, SEQ ID NO: 17, SEQ ID NO: 18, SEQ ID NO: 19, SEQ ID NO: 20, or SEQ ID NO: 21.

In another such embodiment, the caninized antibody or antigen binding fragment comprises both a VL CDR1 comprising an amino acid sequence of SEQ ID NO: 13, SEQ ID NO: 14, or SEQ ID NO: 15 and a VL CDR3 comprising an amino acid sequence of SEQ ID NO: 22, SEQ ID NO: 23, SEQ ID NO: 24, SEQ ID NO: 25, or SEQ ID NO: 26. In yet another such embodiment, the caninized antibody or antigen binding fragment comprises both a VL CDR2 comprising an amino acid sequence of SEQ ID NO: 16, SEQ ID NO: 17, SEQ ID NO: 18, SEQ ID NO: 19, SEQ ID NO: 20, or SEQ ID NO: 21 and a VL CDR3 comprising an amino acid sequence of SEQ ID NO: 22, SEQ ID NO: 23, SEQ ID NO: 24, SEQ ID NO: 25, or SEQ ID NO: 26. In still another such embodiment, the caninized antibody or antigen binding fragment comprises a VL CDR1 comprising an amino acid sequence of SEQ ID NO: 13, SEQ ID NO: 14, or SEQ ID NO: 15, a VL CDR2 comprising an amino acid sequence of SEQ ID NO: 16, SEQ ID NO: 17, SEQ ID NO: 18, SEQ ID NO: 19, SEQ ID NO: 20, or SEQ ID NO: 21, and a VL CDR3 comprising an amino acid sequence of SEQ ID NO: 22, SEQ ID NO: 23, SEQ ID NO: 24, SEQ ID NO: 25, or SEQ ID NO: 26.

The present invention further provides caninized antibodies that comprise the amino acid sequence of SEQ ID NO: 40 or that is 90%, 95%, 98%, or 99% identical to the amino acid sequence of SEQ ID NO: 40, SEQ ID NO: 42 or that is 90%, 95%, 98%, or 99% identical to the amino acid sequence of SEQ ID NO: 42, SEQ ID NO: 44 or that is 90%, 95%, 98%, or 99% identical to the amino acid sequence of SEQ ID NO: 44, SEQ ID NO: 46 or that is 90%, 95%, 98%, or 99% identical to the amino acid sequence of SEQ ID NO: 46, SEQ ID NO: 48 or that is 90%, 95%, 98%, or 99% identical to the amino acid sequence of SEQ ID NO: 48, SEQ ID NO: 50 or that is 90%, 95%, 98%, or 99% identical to the amino acid sequence of SEQ ID NO: 50, SEQ ID NO: 52 or that is 90%, 95%, 98%, or 99% identical to the amino acid sequence of SEQ ID NO: 52, SEQ ID NO: 54 or that is 90%, 95%, 98%, or 99% identical to the amino acid sequence of SEQ ID NO: 54, SEQ ID NO: 56 or that is 90%, 95%, 98%, or 99% identical to the amino acid sequence of SEQ ID NO: 56, SEQ ID NO: 58 or that is 90%, 95%, 98%, or 99% identical to the amino acid sequence of SEQ ID NO: 58, SEQ ID NO: 60 or that is 90%, 95%, 98%, or 99% identical to the amino acid sequence of SEQ ID NO: 60, SEQ ID NO: 62 or that is 90%, 95%, 98%, or 99% identical to the amino acid sequence of SEQ ID NO: 62, SEQ ID NO: 64 or that is 90%, 95%, 98%, or 99% identical to the amino acid sequence of SEQ ID NO: 64, or SEQ ID NO: 66 or that is 90%, 95%, 98%, or 99% identical to the amino acid sequence of SEQ ID NO: 66, or antigen binding fragments of these caninized antibodies.

In particular embodiments, the heavy chain of an antibody comprises the amino acid sequence of SEQ ID NO: 40, 52, 56, or 64 (or 90%, 95%, 98%, or 99% identical to SEQ ID NO: 40, 52, 56, or 64) that comprises (i) P, A, G, or S at position 239, (ii) A, G, or S at position 266, (iii) A, G, or S at position 298, (iv) G, P, or A at position 299, (v) T, A, G, or S at position 300, (vi) A, G, or S at position 328, and (vii) P, A, G, or S at position 330. In other embodiments, the heavy chain of an antibody comprises the amino acid sequence of SEQ ID NO: 42, 54, 58, or 66 (or 90%, 95%, 98%, or 99% identical to SEQ ID NO: 42, 54, 58, or 66) that comprises (i) P, A, G, or S at position 237, (ii) A, G, or S at position 264, (iii) A, G, or S at position 296, (iv) G, P, or A at position 297, (v) T, A, G, or S at position 298, (vi) A, G, or S at position 326, and (vii) P, A, G, or S at position 328. In yet other embodiments, the heavy chain of an antibody comprises the amino acid sequence of SEQ ID NO: 44, 50, or 60 (or 90%, 95%, 98%, or 99% identical to SEQ ID NO: 44, 50, or 60) that comprises (i) P, A, G, or S at position 244, (ii) A, G, or S at position 271, (iii) A, G, or S at position 303, (iv) G, P, or A at position 304, (v) T, A, G, or S at position 305, (vi) A, G, or S at position 333, and (vii) P, A, G, or S at position 335. In still other embodiments, the heavy chain of an antibody comprises the amino acid sequence of SEQ ID NO: 46 or 62 (or 90%, 95%, 98%, or 99% identical to SEQ ID NO: 46 or 62) that comprises (i) P, A, G, or S at position 242, (ii) A, G, or S at position 269, (iii) A, G, or S at position 301, (iv) G, P, or A at position 302, (v) T, A, G, or S at position 303, (vi) A, G, or S at position 331, and (vii) P, A, G, or S at position 333. In yet other embodiments, the heavy chain of an antibody comprises the amino acid sequence of SEQ ID NO: 48 (or 90%, 95%, 98%, or 99% identical to SEQ ID NO: 48) that comprises (i) P, A, G, or S at position 246, (ii) A, G, or S at position 273, (iii) A, G, or S at position 305, (iv) G, P, or A at position 306, (v) T, A, G, or S at position 307, (vi) A, G, or S at position 335, and (vii) P, A, G, or S at position 337.

In still other embodiments, the heavy chain of an antibody comprises the amino acid sequence of SEQ ID NO: 40, 52, 56, or 64 (or 90%, 95%, 98%, or 99% identical to SEQ ID NO: 40, 52, 56, or 64) that comprises (i) P, A, G, or S at position 239, (ii) A at position 266, (iii) A at position 298, (iv) G, P, or A at position 299, (v) T, A, G, or S at position 300, (vi) A, G, or S at position 328, and (vii) P, A, G, or S at position 330. In other embodiments, the heavy chain of an antibody comprises the amino acid sequence of SEQ ID NO: 42, 54, 58, or 66 (or 90%, 95%, 98%, or 99% identical to SEQ ID NO: 42, 54, 58, or 66) that comprises (i) P, A, G, or S at position 237, (ii) A at position 264, (iii) A at position 296, (iv) G, P, or A at position 297, (v) T, A, G, or S at position 298, (vi) A, G, or S at position 326, and (vii) P, A, G, or S at position 328. In yet other embodiments, the heavy chain of an antibody comprises the amino acid sequence of SEQ ID NO: 44, 50, or 60 (or 90%, 95%, 98%, or 99% identical to SEQ ID NO: 44, 50, or 60) that comprises (i) P, A, G, or S at position 244, (ii) A at position 271, (iii) A at position 303, (iv) G, P, or A at position 304, (v) T, A, G, or S at position 305, (vi) A, G, or S at position 333, and (vii) P, A, G, or S at position 335. In still other embodiments, the heavy chain of an antibody comprises the amino acid sequence of SEQ ID NO: 46 or 62 (or 90%, 95%, 98%, or 99% identical to SEQ ID NO: 46 or 62) that comprises (i) P, A, G, or S at position 242, (ii) A at position 269, (iii) A at position 301, (iv) G, P, or A at position 302, (v) T, A, G, or S at position 303, (vi) A, G, or S at position 331, and (vii) P, A, G, or S at position 333. In yet other embodiments, the heavy chain of an antibody comprises the amino acid sequence of SEQ ID NO: 48 (or 90%, 95%, 98%, or 99% identical to SEQ ID NO: 48) that comprises (i) P, A, G, or S at position 246, (ii) A at position 273, (iii) A at position 305, (iv) G, P, or A at position 306, (v) T, A, G, or S at position 307, (vi) A, G, or S at position 335, and (vii) P, A, G, or S at position 337.

In still other embodiments, the heavy chain of an antibody comprises the amino acid sequence of SEQ ID NO: 40, 52, 56, or 64 (or 90%, 95%, 98%, or 99% identical to SEQ ID NO: 40, 52, 56, or 64) that comprises (i) A at position 239, (ii) A at position 266, (iii) A at position 298, (iv) P at position 299, (v) A at position 300, (vi) G, at position 328, and (vii) A, at position 330. In other embodiments, the heavy chain of an antibody comprises the amino acid sequence of SEQ ID NO: 42, 54, 58, or 66 (or 90%, 95%, 98%, or 99% identical to SEQ ID NO: 42, 54, 58, or 66) that comprises (i) A at position 237, (ii) A at position 264, (iii) A at position 296, (iv) P at position 297, (v) A at position 298, (vi) G at position 326, and (vii) A at position 328. In yet other embodiments, the heavy chain of an antibody comprises the amino acid sequence of SEQ ID NO: 44, 50, or 60 (or 90%, 95%, 98%, or 99% identical to SEQ ID NO: 44, 50, or 60) that comprises (i) A at position 244, (ii) A at position 271, (iii) A at position 303, (iv) P at position 304, (v) A at position 305, (vi) G at position 333, and (vii) A at position 335. In still other embodiments, the heavy chain of an antibody comprises the amino acid sequence of SEQ ID NO: 46 or 62 (or 90%, 95%, 98%, or 99% identical to SEQ ID NO: 46 or 62) that comprises (i) A at position 242, (ii) A at position 269, (iii) A at position 301, (iv) P at position 302, (v) A at position 303, (vi) G at position 331, and (vii) A at position 333. In yet other embodiments, the heavy chain of an antibody comprises the amino acid sequence of SEQ ID NO: 48 (or 90%, 95%, 98%, or 99% identical to SEQ ID NO: 48) that comprises (i) A at position 246, (ii) A at position 273, (iii) A at position 305, (iv) P at position 306, (v) A at position 307, (vi) G at position 335, and (vii) A at position 337.

In yet other embodiments, the heavy chain of an antibody comprises the amino acid sequence of SEQ ID NO: 40, 52, 56, or 64 (or 90%, 95%, 98%, or 99% identical to SEQ ID NO: 40, 52, 56, or 64) that comprises (i) P at position 239, (ii) A, G, or S at position 266, (iii) A, G, or S at position 298, (iv) G at position 299, (v) T at position 300, (vi) A at position 328, and (vii) P at position 330. In other embodiments, the heavy chain of an antibody comprises the amino acid sequence of SEQ ID NO: 42, 54, 58, or 66 (or 90%, 95%, 98%, or 99% identical to SEQ ID NO: 42, 54, 58, or 66) that comprises (i) P at position 237, (ii) A, G, or S at position 264, (iii) A, G, or S at position 296, (iv) G at position 297, (v) T at position 298, (vi) A at position 326, and (vii) P at position 328. In yet other embodiments, the heavy chain of an antibody comprises the amino acid sequence of SEQ ID NO: 44, 50, or 60 (or 90%, 95%, 98%, or 99% identical to SEQ ID NO: 44, 50, or 60) that comprises (i) P at position 244, (ii) A, G, or S at position 271, (iii) A, G, or S at position 303, (iv) G at position 304, (v) T at position 305, (vi) A at position 333, and (vii) P at position 335. In still other embodiments, the heavy chain of an antibody comprises the amino acid sequence of SEQ ID NO: 46 or 62 (or 90%, 95%, 98%, or 99% identical to SEQ ID NO: 46 or 62) that comprises (i) P at position 242, (ii) A, G, or S at position 269, (iii) A, G, or S at position 301, (iv) G at position 302, (v) T at position 303, (vi) A at position 331, and (vii) P at position 333. In yet other embodiments, the heavy chain of an antibody comprises the amino acid sequence of SEQ ID NO: 48 (or 90%, 95%, 98%, or 99% identical to SEQ ID NO: 48) that comprises (i) P at position 246, (ii) A, G, or S at position 273, (iii) A, G, or S at position 305, (iv) G at position 306, (v) T at position 307, (vi) A at position 335, and (vii) P at position 337.

In still other embodiments, the heavy chain of an antibody comprises the amino acid sequence of SEQ ID NO: 40, 52, 56, or 64 (or 90%, 95%, 98%, or 99% identical to SEQ ID NO: 40, 52, 56, or 64) that comprises (i) P at position 239, (ii) A at position 266, (iii) A at position 298, (iv) G at position 299, (v) T at position 300, (vi) A at position 328, and (vii) P at position 330. In other embodiments, the heavy chain of an antibody comprises the amino acid sequence of SEQ ID NO: 42, 54, 58, or 66 (or 90%, 95%, 98%, or 99% identical to SEQ ID NO: 42, 54, 58, or 66) that comprises (i) P at position 237, (ii) A at position 264, (iii) A at position 296, (iv) G at position 297, (v) T at position 298, (vi) A at position 326, and (vii) P at position 328. In yet other embodiments, the heavy chain of an antibody comprises the amino acid sequence of SEQ ID NO: 44, 50, or 60 (or 90%, 95%, 98%, or 99% identical to SEQ ID NO: 44, 50, or 60) that comprises (i) P at position 244, (ii) A at position 271, (iii) A at position 303, (iv) G at position 304, (v) T at position 305, (vi) A at position 333, and (vii) P at position 335. In still other embodiments, the heavy chain of an antibody comprises the amino acid sequence of SEQ ID NO: 46 or 62 (or 90%, 95%, 98%, or 99% identical to SEQ ID NO: 46 or 62) that comprises (i) P at position 242, (ii) A at position 269, (iii) A at position 301, (iv) G at position 302, (v) T at position 303, (vi) A at position 331, and (vii) P at position 333. In yet other embodiments, the heavy chain of an antibody comprises the amino acid sequence of SEQ ID NO: 48 (or 90%, 95%, 98%, or 99% identical to SEQ ID NO: 48) that comprises (i) P at position 246, (ii) A at position 273, (iii) A at position 305, (iv) G at position 306, (v) T at position 307, (vi) A at position 335, and (vii) P at position 337.

In other embodiments, the heavy chain of an antibody comprises the amino acid sequence of SEQ ID NO: 40, 52, 56, or 64 (or 90%, 95%, 98%, or 99% identical to SEQ ID NO: 40, 52, 56, or 64) that comprises (i) P, A, G, or S at position 239, (ii) A, G, or S at position 266, (iii) A, G, or S at position 298, (iv) G at position 299, (v) T at position 300, (vi) A, G, or S at position 328, and (vii) P, A, G, or S at position 330. In other such embodiments, the heavy chain of an antibody comprises the amino acid sequence of SEQ ID NO: 42, 54, 58, or 66 (or 90%, 95%, 98%, or 99% identical to SEQ ID NO: 42, 54, 58, or 66) that comprises (i) P, A, G, or S at position 237, (ii) A, G, or S at position 264, (iii) A, G, or S at position 296, (iv) G at position 297, (v) T at position 298, (vi) A, G, or S at position 326, and (vii) P, A, G, or S at position 328. In yet other embodiments, the heavy chain of an antibody comprises the amino acid sequence of SEQ ID NO: 44, 50, or 60 (or 90%, 95%, 98%, or 99% identical to SEQ ID NO: 44, 50, or 60) that comprises (i) P, A, G, or S at position 244, (ii) A, G, or S at position 271, (iii) A, G, or S at position 303, (iv) G at position 304, (v) T at position 305, (vi) A, G, or S at position 333, and (vii) P, A, G, or S at position 335. In still other embodiments, the heavy chain of an antibody comprises the amino acid sequence of SEQ ID NO: 46 or 62 (or 90%, 95%, 98%, or 99% identical to SEQ ID NO: 46 or 62) that comprises (i) P, A, G, or S at position 242, (ii) A, G, or S at position 269, (iii) A, G, or S at position 301, (iv) G at position 302, (v) T at position 303, (vi) A, G, or S at position 331, and (vii) P, A, G, or S at position 333. In yet other embodiments, the heavy chain of an antibody comprises the amino acid sequence of SEQ ID NO: 48 (or 90%, 95%, 98%, or 99% identical to SEQ ID NO: 48) that comprises (i) P, A, G, or S at position 246, (ii) A, G, or S at position 273, (iii) A, G, or S at position 305, (iv) G at position 306, (v) T at position 307, (vi) A, G, or S at position 335, and (vii) P, A, G, or S at position 337.

In yet other embodiments, the heavy chain of an antibody comprises the amino acid sequence of SEQ ID NO: 40, 52, 56, or 64 (or 90%, 95%, 98%, or 99% identical to SEQ ID NO: 40, 52, 56, or 64) that comprises (i) P, A, G, or S at position 239, (ii) A at position 266, (iii) A at position 298, (iv) G at position 299, (v) T at position 300, (vi) A, G, or S at position 328, and (vii) P, A, G, or S at position 330. In other such embodiments, the heavy chain of an antibody comprises the amino acid sequence of SEQ ID NO: 42, 54, 58, or 66 (or 90%, 95%, 98%, or 99% identical to SEQ ID NO: 42, 54, 58, or 66) that comprises (i) P, A, G, or S at position 237, (ii) A at position 264, (iii) A at position 296, (iv) G at position 297, (v) T at position 298, (vi) A, G, or S at position 326, and (vii) P, A, G, or S at position 328. In yet other embodiments, the heavy chain of an antibody comprises the amino acid sequence of SEQ ID NO: 44, 50, or 60 (or 90%, 95%, 98%, or 99% identical to SEQ ID NO: 44, 50, or 60) that comprises (i) P, A, G, or S at position 244, (ii) A at position 271, (iii) A at position 303, (iv) G at position 304, (v) T at position 305, (vi) A, G, or S at position 333, and (vii) P, A, G, or S at position 335. In still other embodiments, the heavy chain of an antibody comprises the amino acid sequence of SEQ ID NO: 46 or 62 (or 90%, 95%, 98%, or 99% identical to SEQ ID NO: 46 or 62) that comprises (i) P, A, G, or S at position 242, (ii) A at position 269, (iii) A at position 301, (iv) G at position 302, (v) T at position 303, (vi) A, G, or S at position 331, and (vii) P, A, G, or S at position 333. In yet other embodiments, the heavy chain of an antibody comprises the amino acid sequence of SEQ ID NO: 48 (or 90%, 95%, 98%, or 99% identical to SEQ ID NO: 48) that comprises (i) P, A, G, or S at position 246, (ii) A at position 273, (iii) A at position 305, (iv) G at position 306, (v) T at position 307, (vi) A, G, or S at position 335, and (vii) P, A, G, or S at position 337.

In yet other embodiments, the heavy chain of an antibody comprises the amino acid sequence of SEQ ID NO: 40, 52, 56, or 64 (or 90%, 95%, 98%, or 99% identical to SEQ ID NO: 40, 52, 56, or 64) that comprises (i) A at position 239, (ii) A at position 266, (iii) A at position 298, (iv) G at position 299, (v) T at position 300, (vi) G at position 328, and (vii) A at position 330. In other such embodiments, the heavy chain of an antibody comprises the amino acid sequence of SEQ ID NO: 42, 54, 58, or 66 (or 90%, 95%, 98%, or 99% identical to SEQ ID NO: 42, 54, 58, or 66) that comprises (i) A at position 237, (ii) A at position 264, (iii) A at position 296, (iv) G at position 297, (v) T at position 298, (vi) G at position 326, and (vii) A at position 328. In yet other embodiments, the heavy chain of an antibody comprises the amino acid sequence of SEQ ID NO: 44, 50, or 60 (or 90%, 95%, 98%, or 99% identical to SEQ ID NO: 44, 50, or 60) that comprises (i) A at position 244, (ii) A at position 271, (iii) A at position 303, (iv) G at position 304, (v) T at position 305, (vi) G at position 333, and (vii) A at position 335. In still other embodiments, the heavy chain of an antibody comprises the amino acid sequence of SEQ ID NO: 46 or 62 (or 90%, 95%, 98%, or 99% identical to SEQ ID NO: 46 or 62) that comprises (i) A at position 242, (ii) A at position 269, (iii) A at position 301, (iv) G at position 302, (v) T at position 303, (vi) G at position 331, and (vii) A at position 333. In yet other embodiments, the heavy chain of an antibody comprises the amino acid sequence of SEQ ID NO: 48 (or 90%, 95%, 98%, or 99% identical to SEQ ID NO: 48) that comprises (i) A at position 246, (ii) A at position 273, (iii) A at position 305, (iv) G at position 306, (v) T at position 307, (vi) G at position 335, and (vii) A at position 337.

In addition, the present invention provides caninized antibody or antigen binding fragment thereof that further comprise a canine light chain that comprises the amino acid sequence of SEQ ID NO: 72, SEQ ID NO: 78, SEQ ID NO: 84, SEQ ID NO: 90, SEQ ID NO: 96, SEQ ID NO: 102, or SEQ ID NO: 108.

Accordingly, the present invention further provides a caninized antibody or antigen binding fragment thereof that comprises a heavy chain comprising the amino acid sequence of SEQ ID NO: 68 and a light chain comprising the amino acid sequence of SEQ ID NO: 72. In a related embodiment, the caninized antibody or antigen binding fragment thereof comprises a heavy chain comprising the amino acid sequence of SEQ ID NO: 70 and a light chain comprising the amino acid sequence of SEQ ID NO: 72. In another embodiment, the caninized antibody or antigen binding fragment thereof comprises a heavy chain comprising the amino acid sequence of SEQ ID NO: 74 and a light chain comprising the amino acid sequence of SEQ ID NO: 78. In a related embodiment, the caninized antibody or antigen binding fragment thereof comprises a heavy chain comprising the amino acid sequence of SEQ ID NO: 76 and a light chain comprising the amino acid sequence of SEQ ID NO: 78. In yet another embodiment, the caninized antibody or antigen binding fragment thereof comprises a heavy chain comprising the amino acid sequence of SEQ ID NO: 80 and a light chain comprising the amino acid sequence of SEQ ID NO: 84. In a related embodiment, the caninized antibody or antigen binding fragment thereof comprises a heavy chain comprising the amino acid sequence of SEQ ID NO: 82 and a light chain comprising the amino acid sequence of SEQ ID NO: 84. In still another embodiment, the caninized antibody or antigen binding fragment thereof comprises a heavy chain comprising the amino acid sequence of SEQ ID NO: 86 and a light chain comprising the amino acid sequence of SEQ ID NO: 90. In a related embodiment, the caninized antibody or antigen binding fragment thereof comprises a heavy chain comprising the amino acid sequence of SEQ ID NO: 88 and a light chain comprising the amino acid sequence of SEQ ID NO: 90. In yet another embodiment, the caninized antibody or antigen binding fragment thereof comprises a heavy chain comprising the amino acid sequence of SEQ ID NO: 92 and a light chain comprising the amino acid sequence of SEQ ID NO: 96. In a related embodiment, the caninized antibody or antigen binding fragment thereof comprises a heavy chain comprising the amino acid sequence of SEQ ID NO: 94 and a light chain comprising the amino acid sequence of SEQ ID NO: 96. In still another embodiment, the caninized antibody or antigen binding fragment thereof comprises a heavy chain comprising the amino acid sequence of SEQ ID NO: 98 and a light chain comprising the amino acid sequence of SEQ ID NO: 102. In a related embodiment, the caninized antibody or antigen binding fragment thereof comprises a heavy chain comprising the amino acid sequence of SEQ ID NO: 100 and a light chain comprising the amino acid sequence of SEQ ID NO: 102. In yet another embodiment, the caninized antibody or antigen binding fragment thereof comprises a heavy chain comprising the amino acid sequence of SEQ ID NO: 104 and a light chain comprising the amino acid sequence of SEQ ID NO: 108. In a related embodiment, the caninized antibody or antigen binding fragment thereof comprises a heavy chain comprising the amino acid sequence of SEQ ID NO: 106 and a light chain comprising the amino acid sequence of SEQ ID NO: 108.

The present invention further provides a caninized antibody or antigen binding fragment thereof that comprises a heavy chain comprising the amino acid sequence of SEQ ID NO: 40 and a light chain comprising the amino acid sequence of SEQ ID NO: 72. In a related embodiment, the caninized antibody or antigen binding fragment thereof comprises a heavy chain comprising the amino acid sequence of SEQ ID NO: 42 and a light chain comprising the amino acid sequence of SEQ ID NO: 72. In another embodiment, the caninized antibody or antigen binding fragment thereof comprises a heavy chain comprising the amino acid sequence of SEQ ID NO: 44 and a light chain comprising the amino acid sequence of SEQ ID NO: 78. In a related embodiment, the caninized antibody or antigen binding fragment thereof comprises a heavy chain comprising the amino acid sequence of SEQ ID NO: 46 and a light chain comprising the amino acid sequence of SEQ ID NO: 78. In yet another embodiment, the caninized antibody or antigen binding fragment thereof comprises a heavy chain comprising the amino acid sequence of SEQ ID NO: 48 and a light chain comprising the amino acid sequence of SEQ ID NO: 84. In a related embodiment, the caninized antibody or antigen binding fragment thereof comprises a heavy chain comprising the amino acid sequence of SEQ ID NO: 50 and a light chain comprising the amino acid sequence of SEQ ID NO: 84. In still another embodiment, the caninized antibody or antigen binding fragment thereof comprises a heavy chain comprising the amino acid sequence of SEQ ID NO: 52 and a light chain comprising the amino acid sequence of SEQ ID NO: 90. In a related embodiment, the caninized antibody or antigen binding fragment thereof comprises a heavy chain comprising the amino acid sequence of SEQ ID NO: 54 and a light chain comprising the amino acid sequence of SEQ ID NO: 90. In yet another embodiment, the caninized antibody or antigen binding fragment thereof comprises a heavy chain comprising the amino acid sequence of SEQ ID NO: 56 and a light chain comprising the amino acid sequence of SEQ ID NO: 96. In a related embodiment, the caninized antibody or antigen binding fragment thereof comprises a heavy chain comprising the amino acid sequence of SEQ ID NO: 58 and a light chain comprising the amino acid sequence of SEQ ID NO: 96.

In still another embodiment, the caninized antibody or antigen binding fragment thereof comprises a heavy chain comprising the amino acid sequence of SEQ ID NO: 60 and a light chain comprising the amino acid sequence of SEQ ID NO: 102. In a related embodiment, the caninized antibody or antigen binding fragment thereof comprises a heavy chain comprising the amino acid sequence of SEQ ID NO: 62 and a light chain comprising the amino acid sequence of SEQ ID NO: 102. In yet another embodiment, the caninized antibody or antigen binding fragment thereof comprises a heavy chain comprising the amino acid sequence of SEQ ID NO: 64 and a light chain comprising the amino acid sequence of SEQ ID NO: 108. In a related embodiment, the caninized antibody or antigen binding fragment thereof comprises a heavy chain comprising the amino acid sequence of SEQ ID NO: 66 and a light chain comprising the amino acid sequence of SEQ ID NO: 108.

The present invention further provides nucleic acids that encode any of the amino acid sequences of the present invention including the CDRs, cFc regions, the cFc regions with the hinge regions, and the heavy chains, and the light chains of the caninized antibodies of the present invention. The present invention further provides expression vectors that comprise one or more of the nucleic acids of the present invention. The present invention further provides host cells that comprise one or more expression vectors of the present invention and methods for expressing the CDRs, and/or cFc regions, and/or the cFc regions with the hinge regions, and/or the heavy chains, and/or the light chains of the caninized antibodies of the present invention using such host cells. The present invention also provides host cells that have been genetically engineered to express the CDRs, and/or cFc regions, and/or the cFc regions with the hinge regions, and/or the heavy chains, and/or the light chains of the caninized antibodies of the present invention in the absence of such vectors. In particular embodiments, these nucleic acids, expression vectors, polypeptides, or host cells of the invention are useful in methods of making an antibody.

In particular embodiments, the antibody is a recombinant antibody or an antigen binding fragment thereof. In related embodiments, the variable heavy chain domain and variable light chain domain are connected by a flexible linker to form a single-chain antibody.

In particular embodiments, the antibody or antigen binding fragment is a Fab fragment.

In other embodiments, the antibody or antigen binding fragment is a Fab′ fragment. In other embodiments, the antibody or antigen binding fragment is a (Fab′)₂ fragment. In still other embodiments, the antibody or antigen binding fragment is a diabody. In particular embodiments, the antibody or antigen binding fragment is a domain antibody. In particular embodiments, the antibody or antigen binding fragment is a camelized single domain antibody.

In particular embodiments, a caninized murine anti-canine PD-1 antibody or antigen binding fragment increases the immune response of the canine subject being treated.

In certain embodiments when bound to canine PD-1, the caninized antibody or antigen binding fragment thereof binds to at least one amino acid residue within one or more amino acid sequences of the following: SEQ ID NO: 138, SEQ ID NO: 139, SEQ ID NO: 140, SEQ ID NO: 141, of SEQ ID NO: 142, SEQ ID NO: 143, SEQ ID NO: 144, and/or SEQ ID NO: 145.

Furthermore, the present invention provides caninized antibodies to canine PD-1, that comprise variants of the CDRs of the present invention that have the corresponding canonical structures provided herein and/or that bind to the amino acid sequence of SEQ ID NO: 144. In particular embodiments of this type, the dissociation constant (Kd) for caninized antibody-canine PD-1 binding is 1×10⁻⁵ to 1×10⁻¹² M. In more particular embodiments the caninized antibodies to canine PD-1, comprise variants of the CDRs of the present invention that have the corresponding canonical structures provided herein and bind to the amino acid sequence of SEQ ID NO: 145. The present invention therefore includes caninized antibodies and antigen binding fragments thereof that bind canine PD-1 with specificity, that when they are bound to canine PD-1, the antibody binds to at least one amino acid residue within SEQ ID NO: 144. In particular embodiments of this type, the antibodies and antigen binding fragments thereof bind canine PD-1 and block the binding of canine PD-1 to canine Programmed Death Ligand 1 (PD-L1).

Accordingly, in particular embodiments when bound to canine PD-1, the caninized antibody (including the antibodies with one or more variant CDR, e.g., a variant including a conservatively modified variant and/or a variant that comprises a defined canonical structure class) binds to at least one amino acid residue within one or more amino acid sequences of the following: SEQ ID NO: 138, SEQ ID NO: 139, SEQ ID NO: 140, SEQ ID NO: 141, of SEQ ID NO: 142, SEQ ID NO: 143, and/or SEQ ID NO: 145. In even more particular embodiments when bound to canine PD-1, the caninized antibodies or antigen binding fragments thereof bind to one or more amino acid residues of the following arginine residues: R₆₂, R₆₉, R₇₂, R₇₅, and R₉₀ of SEQ ID NO: 114. In specific embodiments when bound to canine PD-1, the caninized antibodies or antigen binding fragments thereof bind to at least one amino acid residue within SEQ ID NO: 145. In more specific embodiments when bound to canine PD-1, the antibodies or antigen binding fragments thereof bind to one or more amino acid residues of the following arginine residues: R₆₂, R₆₉, R₇₂, and R₇₅ of SEQ ID NO: 114. In even more specific embodiments when bound to canine PD-1, the antibodies or antigen binding fragments thereof bind to R₇₅ of SEQ ID NO: 114.

The present invention further provides caninized antibodies or antigen binding fragments thereof that bind to canine PD-1 with a dissociation constant (Kd) that is lower (e.g., 1×10⁻¹³ M, or lower) than 1×10⁻¹² M. In particular embodiments the caninized antibodies or antigen binding fragments thereof bind to canine PD-1 with a dissociation constant of 1×10⁻⁵ M to 1×10⁻¹² M. In more particular embodiments the caninized antibodies or antigen binding fragments thereof bind to canine PD-1 with a dissociation constant of 1×10⁻⁷ M to 1×10⁻¹¹ M. In still more particular embodiments the caninized antibodies or antigen binding fragments thereof bind to canine PD-1 with a dissociation constant of 1×10⁻⁸ M to 1×10⁻¹¹ M. In yet more particular embodiments the caninized antibodies or antigen binding fragments thereof bind to canine PD-1 with a dissociation constant of 1×10⁻⁸ M to 1×10⁻¹⁰ M.

The present invention also provides caninized antibodies or antigen binding fragments thereof that bind to canine PD-1 with an on rate (kₒₙ) that is greater than 1×10⁷ M⁻¹s⁻¹. In particular embodiments the caninized antibodies or antigen binding fragments thereof bind to canine PD-1 with an on rate of 1×10² M⁻¹s⁻¹ to 1×10⁷ M⁻¹s⁻¹. In more particular embodiments the caninized antibodies or antigen binding fragments thereof bind to canine PD-1 with an on rate of 1×10³ M⁻¹s⁻¹ to 1×10⁶ M⁻¹s⁻¹. In still more particular embodiments the caninized antibodies or antigen binding fragments thereof bind to canine PD-1 with an on rate of 1×10³ M⁻¹s⁻¹ to 1×10⁵ M⁻¹s⁻¹. In yet more particular embodiments the caninized antibodies or antigen binding fragments thereof bind to canine PD-1 on rate of 1×10⁴ M⁻¹s⁻¹ to 1×10⁵ M⁻¹s⁻¹.

The present invention further provides caninized antibodies or antigen binding fragments thereof that bind to canine PD-1 with an off rate (k_{off}) slower than 1×10⁻⁷ s⁻¹. In particular embodiments the caninized antibodies or antigen binding fragments thereof bind to canine PD-1 with an off rate of 1×10⁻³ s⁻¹ to 1×10⁻⁸ s⁻¹. In more particular embodiments the caninized antibodies or antigen binding fragments thereof bind to canine PD-1 with an off rate of 1×10⁻⁴ s⁻¹ to 1×10⁻⁷ s⁻¹. In still more particular embodiments the caninized antibodies or antigen binding fragments thereof bind to canine PD-1 with an off rate of 1×10⁻⁵ s⁻¹ to 1×10⁻⁷ s⁻¹.

In related embodiments, the caninized antibodies or antigen binding fragments thereof stimulate antigen-specific memory responses to a tumor or pathogen. In particular embodiments, the caninized antibodies or antigen binding fragments thereof stimulate an antibody response in vivo. In other particular embodiments, the caninized antibodies or antigen binding fragments thereof stimulate an immune response in an animal subject. In more specific embodiments the animal subject is a canine. In a related embodiment, the animal subject is a feline.

Accordingly, any of the caninized antibodies of the present invention can exhibit one, two, three, four, five, or all these properties, i.e., the aforesaid dissociation constants with canine PD-1, the aforesaid on rates for binding with canine PD-1, the aforesaid off rates for dissociating from from the caninized antibody-canine PD-1 binding complex, stimulating an antigen-specific memory responses to a tumor or pathogen, stimulating an antibody response in vivo, and/or stimulating an immune response in an animal subject.

In more particular embodiments the caninized antibodies and antigen binding fragments thereof of the present invention bind canine PD-1 and also block the binding of canine PD-1 to PD-L1. In even more particular embodiments the caninized antibodies and antigen binding fragments thereof of the present invention bind canine PD-1, block the binding of canine PD-1 to PD-L1, and also block the binding of canine PD-1 to PD-L2.

The present invention further provides nucleic acids that encode the caninized murine anti-canine PD-1 antibodies or portions thereof of the present invention. In related embodiments such antibodies or antigen binding fragments can be used for the preparation of a medicament to treat cancer in a canine subject. Alternatively, or in conjunction, the present invention provides for the use of any of the antibodies or antibody fragments of the present invention for diagnostic use. In yet additional embodiments, a kit is provided comprising any of the caninized antibodies or antigen binding fragments disclosed herein.

The present invention further includes pharmaceutical compositions comprising an anti-canine antigen antibody or binding fragment thereof (e.g., an anti-canine PD-1 antibody or antigen binding fragment thereof) together with a pharmaceutically acceptable carrier or diluent. The present invention also provides a method of increasing the activity of an immune cell, comprising administering to a subject (e.g., a canine) in need thereof a therapeutically effective amount of the pharmaceutical composition of the present invention. In certain embodiments the method is used for the treatment of cancer. In other embodiments, the method is used in the treatment of an infection or infectious disease. In still other embodiments, a caninized antibody of the present invention or antigen binding fragment thereof is used as a vaccine adjuvant. In yet another embodiment, a caninized anti-TSLP antibody is administered to a canine to treat atopic dermatitis.

These and other aspects of the present invention will be better appreciated by reference to the following Brief Description of the Drawings and the Detailed Description.

### BRIEF DESCRIPTION OF THE DRAWINGS

FIG. 1 shows the reactivity of caninized monoclonal antibodies (mAbs) against extracellular domain of canine PD-1, as a function of OD 650/490 versus the log mAb (nM). Various caninized mAbs were tested for their binding to extracellular domain of canine PD-1 by ELISA. The four mAbs tested were designated as: 2H9 VH4 IgGB/VL3, 3B6 VH3 IgGB/VL3, 2H9 VH4 IgGB (YZZ1062)/VL3, and 2H9 VH4 IgGB (YZZ1068)/VL3.

FIG. 2 shows the reactivity of caninized mAbs against cell surface-expressed canine PD-1. Various mouse mAbs were tested for their binding to canine PD-1 expressed on CHO cells by CELISA as a function of OD 450/540 versus the log mAb (nM). The six mAbs tested were designated as: 3B6 VH3/VL4, 3B6 VH3/VL1, 3B6 VH3/VL3, 3B6 VH3/VL2, 3B6 VH1/VL1, and 3B6 m-c Chimera.

FIG. 3 shows ligand blockade with caninized mAbs against canine PD-1. Various caninized mAbs were tested for their ability to inhibit binding of PD-1 expressed on CHO cells to PD-L1 as a function of OD 450/540 versus the log mAb (nM). The six mAbs tested were designated as: 3B6 VH3/VL4, 3B6 VH3/VL1, 3B6 VH3/VL3, 3B6 VH3/VL2, 3B6 VH1/VL1, and 3B6 m-c Chimera.

FIG. 4 shows the cytokine secretion induced by caninized mAbs against canine PD-1. Various caninized mAbs and their variants were tested for their ability to induce cytokine secretion from PBMC from healthy dogs.

FIGS. 5A and 5B show the binding of caninized mAbs and their variants (beginning at 1 μg/ml) to Fc_{γ}RI. Various mAbs were tested for their ability to bind to FcRI. Antibodies are designated as: can 2H9 ADCC (1062) VH4/VL3, can 2H9 ADCC mut 1 VH4/VL3, can 2H9 ADCC mut 2 VH4/VL3, can 2H9 IgGD VH4/VL3, can 2H9 VH4/VL3, and can 3B6 VH4/VL4 in FIG. 5A; and can 2H9 ADCC (1059) VH4/VL3, can 2H9 ADCC (1060) VH4/VL3, can 2H9 ADCC (1061) VH4/VL3, can 2H9 IgGB ADCC (1068) VH4/VL3, can 2H9 VH4/VL3, and can 3B6 VH4/VL4 in FIG. 5B.

FIGS. 6A and 6B show the binding of caninized mAbs and their variants (beginning at 1 μg/ml) to C1Q. Various mAbs were tested for their ability to bind to CIQ. Antibodies are designated as: can 2H9 VH4 IgGB ADCC (1062)/VL3, can 2H9 VH4 IgGB ADCC (mut 1)/VL3, can 2H9 VH4 IgGB ADCC (mut 2)/VL3, can 2H9 VH4 IgGD/VL3, can 2H9 VH4/VL3, and can 3B6 VH4/VL4 IgGB in FIG. 6A; and can 2H9 VH4 IgGB ADCC (1059)/VL3, can 2H9 VH4 IgGB ADCC (1060)/VL3, can 2H9 VH4 IgGB ADCC (1061)/VL3, can 2H9 VH4 IgGB ADCC (1068)/VL3, can 2H9 VH4/VL3 IgGB, and can 3B6 VH4/VL4 IgGB in FIG. 6B.

FIG. 7A shows the characterization of the interface between canine PD-1 and the caninized antibody 2G9. The amino acid positions are with respect to the PD-1 amino acid sequence without the signal sequence, i.e., SEQ ID NO: 114. The determination was performed by chemical cross-linking, High-Mass MALDI mass spectrometry and nLC-Orbitrap mass spectrometry.

FIG. 7B shows the characterization of the interface between canine PD-1 and the caninized antibody 3B6. The amino acid positions are with respect to the PD-1 amino acid sequence without the signal sequence, i.e., SEQ ID NO: 114. The determination was performed by chemical cross-linking, High-Mass MALDI mass spectrometry and nLC-Orbitrap mass spectrometry.

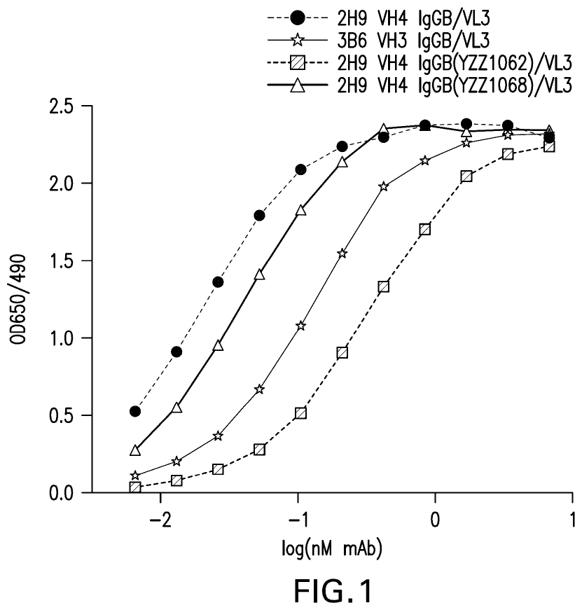

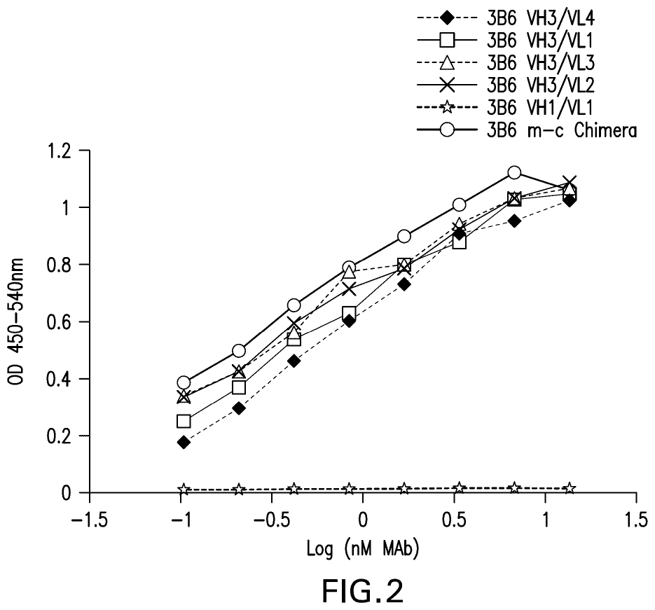

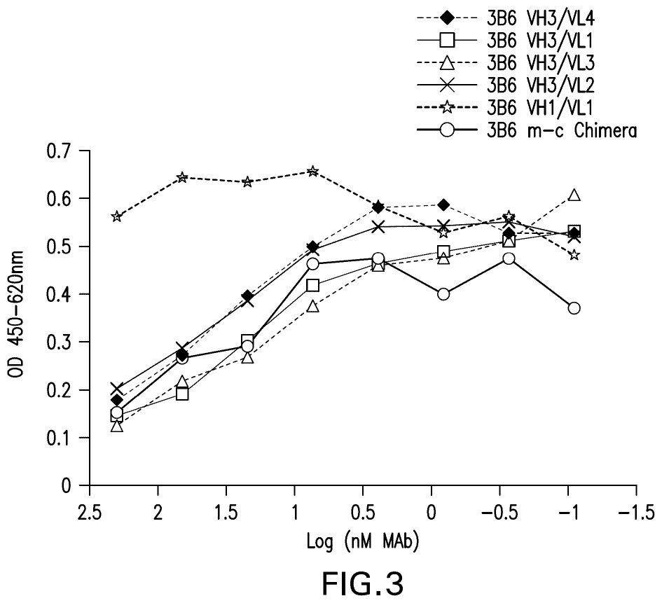

### DETAILED DESCRIPTION

### Abbreviations

Throughout the detailed description and examples of the invention the following abbreviations will be used:
ADCC Antibody-dependent cellular cytotoxicity
CDC Complement-dependent cyotoxicity
CDR Complementarity determining region in the immunoglobulin variable regions, defined for human antibodies using the Kabat numbering system
CHO Chinese hamster ovary
EC50 concentration resulting in 50% efficacy or binding
ELISA Enzyme-linked immunosorbant assay
FR Antibody framework region: the immunoglobulin variable regions excluding the CDR regions.
HRP Horseradish peroxidase
IFN interferon
IC50 concentration resulting in 50% inhibition
IgG Immunoglobulin G
Kabat An immunoglobulin alignment and numbering system for humam antibodies pioneered by Elvin A. Kabat [Sequences of Proteins of Immunological Interest, 5th Ed. Public Health Service, National Institutes of Health, Bethesda, Md. (1991)]
mAb Monoclonal antibody (also Mab or MAb)
MES 2-(N-morpholino) ethanesulfonic acid
MOA Mechanism of action
NHS Normal human serum
PCR Polymerase chain reaction
PK Pharmacokinetics
SEB Staphylococcus Enterotoxin B
TT Tetanus toxoid
V region The segment of human IgG chains which is variable in sequence between different antibodies. It extends to Kabat residue 109 in the light chain and 113 in the heavy chain.
VH Immunoglobulin heavy chain variable region
VK Immunoglobulin kappa light chain variable region

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Activation” as it applies to cells or to receptors refers to the activation or treatment of a cell or receptor with a ligand, unless indicated otherwise by the context or explicitly. “Ligand” encompasses natural and synthetic ligands, e.g., cytokines, cytokine variants, analogues, muteins, and binding compounds derived from antibodies. “Ligand” also encompasses small molecules, e.g., peptide mimetics of cytokines and peptide mimetics of antibodies. “Activation” can refer to cell activation as regulated by internal mechanisms as well as by external or environmental factors.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Activity” may refer to modulation of components of the innate or the adaptive immune systems.

“Administration” and “treatment,” as it applies to an animal, e.g., a canine experimental subject, cell, tissue, organ, or biological fluid, refers to contact of an exogenous pharmaceutical, therapeutic, diagnostic agent, or composition to the animal e.g., a canine subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell.

The term “subject” includes any organism, preferably an animal, more preferably a mammal (e.g., canine, feline, or human) and most preferably a canine.

As used herein, the term “feline” refers to any member of the Felidae family. Members of this family include wild, zoo, and domestic members, such as any member of the subfamilies Felinae, e.g., cats, lions, tigers, pumas, jaguars, leopards, snow leopards, panthers, North American mountain lions, cheetahs, lynx, bobcats, caracals or any cross breeds thereof. Cats also include domestic cats, pure-bred and/or mongrel companion cats, show cats, laboratory cats, cloned cats, and wild or feral cats.

As used herein, a “substitution of an amino acid residue” with another amino acid residue in an amino acid sequence is equivalent to “replacing an amino acid residue” with another amino acid residue and denotes that a particular amino acid residue at a specific position in the amino acid sequence has been replaced by (or substituted for) by a different amino acid. For example, one such substitution (replacement) is denoted as P4A of an Fc region of an IgGB or IgGC amino acid sequence, in which case, the proline residue at amino acid position 4 of the amino acid sequence of the Fc region of an IgGB or the Fc region of an IgGC has been substituted for (replaced) by an alanine residue.

Accordingly, such amino acid substitutions can be particularly designed i.e., purposefully replacing an alanine with a serine at a specific position in the amino acid sequence by e.g., recombinant DNA technology. Alternatively, a particular amino acid residue or string of amino acid residues of an antibody can be replaced by one or more amino acid residues through more natural selection processes e.g., based on the ability of the antibody produced by a cell to bind to a given region on that antigen, e.g., one containing an epitope or a portion thereof, and/or for the antibody to comprise a particular CDR that retains the same canonical structure as the CDR it is replacing. Such substitutions/replacements can lead to “variant” CDRs and/or antibodies.

“Treat” or “treating” means to administer a therapeutic agent, such as a composition containing any of the antibodies or antigen binding fragments of the present invention, internally or externally to a canine subject or patient having one or more disease symptoms, or being suspected of having a disease, for which the agent has therapeutic activity.

Typically, the agent is administered in an amount effective to alleviate and/or ameliorate one or more disease symptom in the treated subject or population, whether by inducing the regression of or inhibiting the progression of such symptom(s) by any clinically measurable degree. The amount of a therapeutic agent that is effective to alleviate any particular disease symptom (also referred to as the “therapeutically effective amount”) may vary according to factors such as the disease state, age, and weight of the subject (e.g., canine), and the ability of the pharmaceutical composition to elicit a desired response in the subject. Whether a disease symptom has been alleviated or ameliorated can be assessed by any clinical measurement typically used by veteranarians or other skilled healthcare providers to assess the severity or progression status of that symptom. While an embodiment of the present invention (e.g., a treatment method or article of manufacture) may not be effective in alleviating the target disease symptom(s) in every subject, it should alleviate the target disease symptom(s) in a statistically significant number of subjects as determined by any statistical test known in the art such as the Student's t-test, the chi2-test, the U-test according to Mann and Whitney, the Kruskal-Wallis test (H-test), Jonckheere-Terpstra-test and the Wilcoxon-test.

“Treatment,” as it applies to a human, veterinary (e.g., canine) or research subject, refers to therapeutic treatment, as well as research and diagnostic applications. “Treatment” as it applies to a human, veterinary (e.g., canine), or research subject, or cell, tissue, or organ, encompasses contact of the caninized antibodies or antigen binding fragments of the present invention to a canine or other animal subject, a cell, tissue, physiological compartment, or physiological fluid.

Canine PD-1 has been found to comprise the amino acid sequence of SEQ ID NO: 114 [U.S. provisional application No. 61/918,946, filed on Dec. 20, 2013, the contents of which are hereby incorporated herein in their entireties]. In a specific embodiment canine PD-1 is encoded by a nucleic acid that comprises the nucleotide sequence of SEQ ID NO: 113.

Canine PD-L1 has been found to comprise the amino acid sequence of SEQ ID NO: 120 [U.S. provisional application No. 61/918,946, filed on Dec. 20, 2013, supra]. In a specific embodiment canine PD-L1 is encoded by a nucleotide sequence comprising SEQ ID NO: 119.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the mammalian body (e.g., canine body) of cancerous cells, cells or tissues infected with pathogens, or invading pathogens.

### Caninized Anti-Canine Antigen Antibodies

As used herein, the term “canine” includes all domestic dogs, Canis lupus familiaris or Canis familiaris, unless otherwise indicated.

As used herein, an antibody is said to bind specifically to a polypeptide comprising a given antigen sequence (in this case a portion of the amino acid sequence of a canine antigen, e.g., canine PD-1) if it binds to polypeptides comprising that portion of the amino acid sequence of the canine antigen, e.g., canine PD-1, but does not bind to other canine proteins lacking that portion of the sequence of the canine antigen, e.g., canine PD-1. For example, an antibody that specifically binds to a polypeptide comprising canine PD-1 may bind to a FLAG®-tagged form of canine PD-1, but will not bind to other FLAG®-tagged canine proteins with specificity. An antibody, or binding compound derived from the antigen-binding site of an antibody, binds to its canine antigen, or a variant or mutein thereof, “with specificity” when it has an affinity for that canine antigen or a variant or mutein thereof which is at least ten-times greater, more preferably at least 20-times greater, and even more preferably at least 100-times greater than its affinity for any other canine antigen tested.

As used herein, the term “antibody” refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), caninized antibodies, fully canine antibodies, chimeric antibodies and camelized single domain antibodies. “Parental antibodies” are antibodies obtained by exposure of an immune system to an antigen prior to modification of the antibodies for an intended use, such as caninization of an antibody for use as a canine therapeutic antibody.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antigen binding fragments include, but are not limited to, Fab, Fab′, F(ab′)2, and Fv fragments; diabodies; linear antibodies; single-chain antibody molecules, e.g., sc-Fv; nanobodies and multispecific antibodies formed from antibody fragments.

A “Fab fragment” is comprised of one light chain and the CH1 and variable regions of one heavy chain. The heavy chain of a Fab molecule cannot form a disulfide bond with another heavy chain molecule. A “Fab fragment” can be the product of papain cleavage of an antibody.

A “fragment crystallizable” (“Fc”) region contains two heavy chain fragments (i.e., two identical polypeptides) comprising the CH2 and CH3 domains of an antibody. The two heavy chain fragments are held together by two or more disulfide bonds and by hydrophobic interactions of the CH3 domains. In the present invention, the amino acid sequence for each of the four canine IgG Fc fragments is based on the identified boundary of CH1 and CH2 domains as determined by Tang et al. [Vet. Immunol. Immunopathol. 80:259-270 (2001)].

A “Fab′ fragment” contains one light chain and a portion or fragment of one heavy chain that contains the VH domain and the CH1 domain and also the region between the CH1 and CH2 domains, such that an interchain disulfide bond can be formed between the two heavy chains of two Fab′ fragments to form a F(ab′)2 molecule.

A “F(ab′)2 fragment” contains two light chains and two heavy chains containing a portion of the constant region between the CH1 and CH2 domains, such that an interchain disulfide bond is formed between the two heavy chains. A F(ab′)2 fragment thus is composed of two Fab′ fragments that are held together by a disulfide bond between the two heavy chains. An “F(ab′)2 fragment” can be the product of pepsin cleavage of an antibody.

The “Fv region” comprises the variable regions from both the heavy and light chains, but lacks the constant regions.

The term “single-chain Fv” or “scFv” antibody refers to antibody fragments comprising the VH and VL domains of an antibody, wherein these domains are present in a single polypeptide chain. Generally, the Fv polypeptide further comprises a polypeptide linker between the VH and VL domains which enables the scFv to form the desired structure for antigen binding. [See, Pluckthun, The Pharmacology of Monoclonal Antibodies, vol. 113 Rosenburg and Moore eds., Springer-Verlag, New York, pp. 269-315 (1994); WO 88/01649; and U.S. Pat. Nos. 4,946,778 and 5,260,203.]

As used herein, the term “canonical structure” refers to the local conformation that can be adopted by each of the hypervariable regions of the heavy and light chain of an antibody within the framework that they reside. For each hypervariable region, there are a small number of canonical structures (generally denoted by simple integers such as 1 or 2 etc.), which can be predicted with great accuracy from the amino acid sequences of the corresponding hypervariable region (particularly within the context of the amino acid sequence of its framework, as provided below for the corresponding caninized murine anti-canine PD-1 variable domains). These canonical structures can be determinative regarding whether a modification of the amino acid sequence of a given CDR will result in the retention or loss of the ability to bind to its antigen binding partner [See, Chothia and Lesk, Canonical Structures for the hypervariable regions of immunoglobulins, J. Mol. Biol. 196:901-917 (1987); Chothia et al., Conformation of immunoglobulin hypervaribale regions, Nature, 34:877-883 (1989); and Al-Lazikani et al., Standard Conformations for the canonical structures of immunoglobulins, J. Mol. Biol. 273:927-948 (1997)].

A “domain antibody” is an immunologically functional immunoglobulin fragment containing only the variable region of a heavy chain or the variable region of a light chain. In some instances, two or more VH regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific (see below).

In certain embodiments, monoclonal antibodies herein also include camelized single domain antibodies. [See, e.g., Muyldermans et al., Trends Biochem. Sci. 26:230 (2001); Reichmann et al., J. Immunol. Methods 231:25 (1999); WO 94/04678; WO 94/25591; U.S. Pat. No. 6,005,079]. In one embodiment, the present invention provides single domain antibodies comprising two VH domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. [See, EP 0 404 097 B1; WO 93/11161; and Holliger et al., Proc. Natl. Acad. Sci. USA 90:6444-6448 (1993)]. For a review of engineered antibody variants [generally see Holliger and Hudson Nat. Biotechnol. 23:1126-1136 (2005)].

Typically, an antibody or antigen binding fragment of the invention retains at least 10% of its canine PD-1 binding activity (when compared to the parental antibody) when that activity is expressed on a molar basis. Preferably, an antibody or antigen binding fragment of the invention retains at least 20%, 50%, 70%, 80%, 90%, 95% or 100% or more of the canine antigen, (e.g., PD-1) binding affinity as the parental antibody. It is also intended that a caninized antibody or antigen binding fragment of the invention can include conservative or non-conservative amino acid substitutions (referred to as “conservative variants” or “function conserved variants” of the antibody) that do not substantially alter its biologic activity.

“Isolated antibody” refers to the purification status and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

As used herein, a “chimeric antibody” is an antibody having the variable domain from a first antibody and the constant domain from a second antibody, where the first and second antibodies are from different species. [U.S. Pat. No. 4,816,567; and Morrison et al., Proc. Natl. Acad. Sci. USA 81:6851-6855 (1984)]. Typically the variable domains are obtained from an antibody from an experimental animal (the “parental antibody”), such as a rodent, and the constant domain sequences are obtained from the animal subject antibodies, e.g., canine, so that the resulting chimeric antibody will be less likely to elicit an adverse immune response in a canine subject, than the parental (e.g., rodent) antibody.

As used herein, the term “caninized antibody” refers to forms of antibodies that contain sequences from both canine and non-canine (e.g., murine) antibodies. In general, the caninized antibody will comprise substantially all of at least one, and typically two, variable domains, in which all or substantially all of the hypervariable loops correspond to those of a non-canine immunoglobulin (e.g., comprising 6 murine anti-canine PD-1 CDRs as exemplified below), and all or substantially all of the canine frame.

The term “fully canine antibody” refers to an antibody that comprises canine immunoglobulin protein sequences only. A fully canine antibody may contain murine carbohydrate chains if produced in a mouse, in a mouse cell, or in a hybridoma derived from a mouse cell. Similarly, “mouse antibody” refers to an antibody that comprises mouse immunoglobulin sequences only. Alternatively, a fully canine antibody may contain rat carbohydrate chains if produced in a rat, in a rat cell, or in a hybridoma derived from a rat cell. Similarly, “rat antibody” refers to an antibody that comprises rat immunoglobulin sequences only.

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), located within relatively conserved framework regions (FR). The CDRs are usually flanked by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain for human antibodies is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat, Adv. Prot. Chem. 32:1-75 (1978); Kabat, et al., J. Biol. Chem. 252:6609-6616 (1977); Chothia, et al., J. Mol. Biol. 196:901-917 (1987) or Chothia, et al., Nature 342:878-883 (1989)].

As used herein, the term “hypervariable region” refers to the amino acid residues of an antibody that are responsible for antigen-binding. The hypervariable region comprises amino acid residues from a “complementarity determining region” or “CDR” (i.e. CDRL1, CDRL2 and CDRL3 in the light chain variable domain and CDRH1, CDRH2 and CDRH3 in the heavy chain variable domain). [See Kabat et al. Sequences of Proteins of Immunological Interest, 5th Ed. Public Health Service, National Institutes of Health, Bethesda, Md. (1991), definining the CDR regions of a human antibody by sequence; see also Chothia and Lesk, J. Mol. Biol. 196:901-917 (1987) defining the CDR regions of an antibody by structure]. As used herein, the term “framework” or “FR” residues refers to those variable domain residues other than the hypervariable region residues defined herein as CDR residues.

As used herein the term “canine frame” refers to the amino acid sequence of the heavy chain and light chain of a canine antibody other than the hypervariable region residues defined herein as CDR residues. In both chains, the amino acid sequences of the native canine CDRs are replaced with the corresponding foreign CDRs (e.g., those from a mouse antibody). Optionally the heavy and/or light chains of the canine antibody may contain some foreign non-CDR residues, e.g., so as to preserve the conformation of the foreign CDRs within the canine antibody, and/or to modify the Fc function, as exemplified below.

As used herein, an “anti-canine PD-1 antibody” refers to an antibody that was raised against canine PD-1 (in a mammal such as a mouse or rabbit) and that specifically binds to canine PD-1. An antibody that “specifically binds to canine PD-1,” or an antibody that “specifically binds to a polypeptide comprising the amino acid sequence of SEQ ID NO: 114”, is an antibody that exhibits preferential binding to canine PD-1 as compared to other antigens, e.g., binds canine PD-1 “with specificity”. The binding does not require absolute binding specificity. An anti-canine PD-1 antibody is considered “specific” for canine PD-1 if its binding is determinative of the presence of canine PD-1 in a sample, or if it is capable of altering the activity of canine PD-1 without unduly interfering with the activity of other molecules in a canine sample, e.g., without producing undesired results such as false positives in a diagnostic context or side effects in a therapeutic context. The degree of specificity necessary for an anti-canine PD-1 antibody may depend on the intended use of the antibody, and at any rate is defined by its suitability for use for an intended purpose.

Accordingly the present invention provides caninized anti-canine PD-1 antibodies or antigen binding fragments thereof (including in isolated form) that bind canine PD-1 (e.g., with specificity) and uses of such antibodies or fragments thereof. In specific embodiments murine anti-canine PD-1 CDRs from murine anti-canine PD-1 antibodies are provided that have been shown to both bind canine PD-1 and to block the binding of canine PD-1 to at least one of its ligands, e.g., canine PD-L1. These CDRs can be inserted into a modified canine frame of the present invention to make a caninized murine anti-canine PD-1 antibody, as exemplified herein.

More specifically, a “caninized murine anti-PD-1 antibody” of the present invention refers to an antibody that comprises the three heavy chain CDRs and the three light chain CDRs from a murine anti-canine PD-1 antibody together with a canine frame or a modified canine frame. A modified canine frame comprises one or more amino acids changes as exemplified herein that further optimize the effectiveness of the caninized antibody, e.g., to augment, reduce, or eliminate antibody effector functions, to increase its binding to the canine antigen, e.g., canine PD-1, and/or increase its ability to block the binding of the canine antigen, e.g., canine PD-1, to its natural binding partner, (e.g., canine PD-L1 in the case where the antigen is canine PD-1).

“Homology” refers to sequence similarity between two polynucleotide sequences or between two polypeptide sequences when they are optimally aligned. When a position in both of the two compared sequences is occupied by the same base or amino acid monomer subunit, e.g., if a position in each of two DNA molecules is occupied by adenine, then the molecules are homologous at that position. The percent of homology is the number of homologous positions shared by the two sequences divided by the total number of positions compared×100. For example, if 6 of 10 of the positions in two sequences are matched or homologous when the sequences are optimally aligned then the two sequences are 60% homologous. Generally, the comparison is made when two sequences are aligned to give maximum percent homology.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

As used herein, “germline sequence” refers to a sequence of unrearranged immunoglobulin DNA sequences. Any suitable source of unrearranged immunoglobulin sequences may be used. Human germline sequences may be obtained, for example, from JOINSOLVER® germline databases on the website for the National Institute of Arthritis and Musculoskeletal and Skin Diseases of the United States National Institutes of Health. Mouse germline sequences may be obtained, for example, as described in Giudicelli et al. [Nucleic Acids Res. 33: D256-D261 (2005)].

### Properties of Caninized Antibodies

In canine, there are four IgG heavy chains referred to as A, B, C, and D. These heavy chains represent four different subclasses of dog IgG, which are referred to as IgGA, IgGB, IgGC and IgGD. The DNA and amino acid sequences of these four heavy chains were first identified by Tang et al. [Vet. Immunol. Immunopathol. 80:259-270 (2001)]. The amino acid and DNA sequences for these heavy chains are also available from the GenBank data bases. For example, the amino acid sequence of IgGA heavy chain has accession number AAL35301.1, IgGB has accession number AAL35302.1, IgGC has accession number AAL35303.1, and IgGD has accession number (AAL35304.1). Canine antibodies also contain two types of light chains, kappa and lambda. The DNA and amino acid sequence of these light chains can be obtained from GenBank Databases. For example the kappa light chain amino acid sequence has accession number ABY 57289.1 and the lambda light chain has accession number ABY 55569.1. In the present invention, the amino acid sequence for each of the four canine IgG Fc fragments is based on the identified boundary of CH1 and CH2 domains as determined by Tang et al, supra.

The development of a therapeutic monoclonal antibody is a complex process that entails coordination of a complex set of activities to generate the desired antibody. These include optimization of the antibody specificity, affinity, functional activity, expression level in engineered cell lines, long-term stability, elimination or enhancement of effector functions and development of commercially viable manufacturing and purification methods. Considering the objectives of the present invention and aside from the capacity to activate cells of the immune systems, a caninized or canine monoclonal antibody against canine PD-1 optimally has three additional attributes:
1. lack of effector functions such as antibody-dependent cytotoxicity (ADCC) and complement-dependent cytotoxicity (CDC),
2. relatively long half-life in vivo; and
3. be readily purified on a large scale using industry standard technologies such as that based on protein A chromatography.

None of the naturally occurring canine IgG subclasses satisfy all these criteria. For example, IgGB can be purified using protein A, but has a high level of ADCC activity. IgGC also has considerable ADCC activity. On the other hand, IgGA binds weakly to protein A, but displays undesirable ADCC activity. Moreover, neither IgGC nor IgGD can be purified on protein A columns, although IgGD display no ADCC activity. Additionally IgGC has short serum half-life as it does not bind to the canine FcRn receptor. The present invention overcomes this difficulty by providing modified canine IgG antibodies specific to canine antigens, e.g., canine PD-1; such antibodies lack effector functions such as ADCC and CDC, display relatively long half-life, and can be easily of purified using industry standard protein A chromatography.

Heretofore, genetically modified canine IgGs that lacked both ADCC and CDC effector functions and in addition, could be purified by protein A chromatography had not been previously described. As disclosed herein, a single substitution at a position in canine IgG that is analogous to that of human and mouse IgG, such as N297A or D265A, does not completely eliminate both ADCC and CDC effector functions in the corresponding canine antibody. For example, while each of the substitutions N297 and D265 in human or murine antibodies results in abrogation of binding to Fc receptor and C1q, neither substitution alone completely abrogated the binding of canine antibodies to C1q. Instead, as further disclosed below, in order to eliminate both ADCC and CDC in canine antibodies of IgGB or IgGC sub-classes, it proved necessary to make a double substitution in the Fc of the canine antibody combining both an asparagine-to-alanine and an aspartic acid-to-alanine substitution. Moreover, completely unexpectedly, one substitution that had been shown to reduce effector functions in human antibodies actually resulted in an increase in binding of corresponding canine IgG to Fc_{γ}R and C1q.

In order to generate variants of canine IgGB and IgGC that lack effector functions, modified canine IgGB or modified canine IgGC heavy chains can be generated. A total of seven amino acid residues which are present in both of these canine fragment crystallizable regions (cFcs) were identified for such possible substitution, These seven amino acid residues are: P4, D31, N63, G64, T65, A93, and P95 for both the amino acid sequence of SEQ ID NO: 130 for the Fc of canine IgGB; and the amino acid sequence of SEQ ID NO: 132 for the Fc of canine IgGC. Accordingly, the amino acid sequence of SEQ ID NO: 2 differs from that of SEQ ID NO: 130 by having the amino acid residues at positions: 4, 31, 63, 64, 65, 93, and 95, which are proline (P), aspartic acid (D), asparagine (N), glycine (G), threonine (T), alanine (A), and proline (P), respectively, in the amino acid sequence of SEQ ID NO: 130 as “X” (or “Xaa” in the three letter code) for all seven positions, signifying that these seven amino acid positions can be any of the twenty natural amino acids (see list in column 1 of Table 1 below). Similarly, the amino acid sequence of SEQ ID NO: 4 differs from that of SEQ ID NO: 132 by having the amino acid residues at positions 4, 31, 63, 64, 65, 93, and 95 are listed as “X” (or “Xaa” in the three letter code) for all seven positions, signifying that these seven amino acid positions can be any of the twenty natural amino acids. The amino acid sequence of SEQ ID NO: 2 is encoded by the nucleotide sequence of SEQ ID NO: 1, whereas the amino acid sequence of SEQ ID NO: 4 is encoded by the nucleotide sequence of SEQ ID NO: 3.

In one embodiment, the cFc comprises the amino acid sequence of SEQ ID NO: 130 with the following substitutions P4 (A, G, or S), D31 (A, G, or S) N63 (A, G, or S), G64 (A or P), T65 (A, G, or S), A93 (G or S), and P95 (A, G, or S); in which P4 (A G, or S) signifies that the proline residue at position 4 is replaced by either an alanine, glycine, or serine residue, and similarly G64 (P or A) signifies that the glycine residue at position 64 is replaced by either a proline or an alanine residue, etc.). In a particular embodiment, the cFc comprises the amino acid sequence of SEQ ID NO: 130 with the following substitutions: P4A, D31A, N63A, G64P, T65A, A93G, and P95A.

In a related embodiment, the cFc comprises the amino acid sequence of SEQ ID NO: 4, which contains 7 amino acids designated as Xaa, with the following amino acid residues: A4, A31, A63, G64, T65, G93, and A95, i.e., the amino acid sequence of SEQ ID NO: 132 with the following five (5) amino acid residue changes: P4A, D31A, N63A, A93G, and P95A and the remaining two amino acid residues of the seven, G64 and T65, being retained from the amino acid sequence of SEQ ID NO: 132.

The amino acid sequences of SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, and SEQ ID NO: 66 all contain “X” (or “Xaa” in the three letter code) at seven amino acid positions, signifying that these seven amino acid positions can be any of the twenty natural amino acids listed in column 1 of Table 1 below. Notably SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, and SEQ ID NO: 66 comprise either the amino acid sequence of SEQ ID NO: 2 or that of SEQ ID NO: 4 within their respective sequences. Specific examples of the amino acid residues at one or more of these seven positions of the amino acid sequences are delineated above and below, and are therefore included within the genus of the individual amino acid sequences of SEQ ID NO: 2, SEQ ID NO: 4, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, and SEQ ID NO: 66, as well as within the caninized antibodies that comprise these sequences.

Table 10 provided below, specifically correlates the seven amino acid positions that can be replaced, as disclosed herein, of the cIgGB Fc (SEQ ID NO: 130 and SEQ ID NO: 2) and the cIgGC Fc (SEQ ID NO: 132 and SEQ ID NO: 4) with that of the full length canine heavy chains that comprises these cFc amino acid sequences, i.e., SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, and SEQ ID NO: 66. Accordingly, the actual position in the full length sequence IgGB or IgGC can be readily coordinated with that of the cFc that it comprises through the use of Table 10 below.

In particular embodiments, the heavy chain of an antibody comprises the amino acid sequence of SEQ ID NO: 40, 52, 56, or 64 comprising (i) P, A, G, or S at position 239, (ii) D, A, G, or S at position 266, (iii) N, A, G, or S at position 298, (iv) G, P, or A at position 299, (v) T, A, G, or S at position 300, (vi) A, G, or S at position 328, and (vii) P, A, G, or S at position 330. In other embodiments, the heavy chain of an antibody comprises the amino acid sequence of SEQ ID NO: 42, 54, 58, or 66 comprising (i) P, A, G, or S at position 237, (ii) D, A, G, or S at position 264, (iii) N, A, G, or S at position 296, (iv) G, P, or A at position 297, (v) T, A, G, or S at position 298, (vi) A, G, or S at position 326, and (vii) P, A, G, or S at position 328. In yet other embodiments, the heavy chain of an antibody comprises the amino acid sequence of SEQ ID NO: 44, 50, or 60 comprising (i) P, A, G, or S at position 244, (ii) D, A, G, or S at position 271, (iii) N, A, G, or S at position 303, (iv) G, P, or A at position 304, (v) T, A, G, or S at position 305, (vi) A, G, or S at position 333, and (vii) P, A, G, or S at position 335. In still other embodiments, the heavy chain of an antibody comprises the amino acid sequence of SEQ ID NO: 46 or 62 comprising (i) P, A, G, or S at position 242, (ii) D, A, G, or S at position 269, (iii) N, A, G, or S at position 301, (iv) G, P, or A at position 302, (v) T, A, G, or S at position 303, (vi) A, G, or S at position 331, and (vii) P, A, G, or S at position 333. In yet other embodiments, the heavy chain of an antibody comprises the amino acid sequence of SEQ ID NO: 48 comprising (i) P, A, G, or S at position 246, (ii) D, A, G, or S at position 273, (iii) N, A, G, or S at position 305, (iv) G, P, or A at position 306, (v) T, A, G, or S at position 307, (vi) A, G, or S at position 335, and (vii) P, A, G, or S at position 337.

The present invention also provides modified canine IgGDs which comprise a hinge region from either IgGA, IgGB, or IgGC in place of its natural IgGD hinge region. Alternatively, the IgGD hinge region can be genetically modified by replacing a serine residue with a proline residue as shown in Table 5. Such modifications can lead to a canine IgGD lacking fab arm exchange. The modified canine IgGDs can be constructed using standard methods of recombinant DNA technology [e.g., Maniatis et al., Molecular Cloning, A Laboratory Manual (1982)]. In order to construct these variants, the nucleic acids encoding the amino acid sequence of canine IgGD can be modified so that it encodes the modified IgGDs. The modified nucleic acid sequences are then cloned into expression plasmids for protein expression. The nucleic acids encoding the canine IgGD Fcs with the substitute hinge region are exemplified by nucleotide sequences of SEQ ID NOs: 7, 9, and 11 which encode the amino acid sequences of SEQ ID NOs: 8, 10, and 12. A nucleic acid encoding a canine IgGD Fc with a modified IgGD hinge region comprises the nucleotide sequence of SEQ ID NO: 5 which encodes the amino acid sequence of SEQ ID NO: 6.

The present invention further provides full length canine heavy chains that can be matched with corresponding light chains to make a caninized antibody. Accordingly, the present invention further provides caninized murine anti-canine antigen antibodies (including isolated caninized murine anti-canine PD-1 antibodies) and methods of use of the antibodies or antigen binding fragments thereof in the treatment of disease e.g., the treatment of cancer in canines.

Moreover, the present invention provides caninized murine anti-canine PD-1 antibodies or antigen binding fragments that bind to canine PD-1 and block the binding of canine PD-1 to canine PD-L1. In certain embodiments the caninized murine anti-canine PD-1 antibodies comprise a modified canine IgGB Fc, modified canine IgGC Fc, or a modified canine IgGD lacking fab arm exchange as described herein.

The antibody or antigen binding fragment thereof that binds the canine antigen, e.g., canine PD-1, can comprise one, two, three, four, five, or six of the complementarity determining regions (CDRs) of the murine anti-canine antibody as described herein. The one, two, three, four, five, or six CDRs may be independently selected from the CDR sequences of those provided below. In a further embodiment, the antibody or antigen-binding fragment thereof that binds canine PD-1 comprises a canine antibody kappa light chain comprising a murine light chain CDR-1, CDR-2 and/or CDR-3 and a canine antibody heavy chain IgG comprising a murine heavy chain CDR-1, CDR-2 and/or CDR-3. Accordingly, the present invention further provides full length canine heavy chains then can be matched e.g., with the corresponding light chains to make a caninized antibody [see Table 2 below, in which the sequences of seven sets of CDRs of murine anti-canine PD-1, e.g., 1B5, 2G9, 2H9, 3B6, 4D12, 5G5, and 7C9 are provided].

In other embodiments, the invention provides antibodies or antigen binding fragments thereof that bind PD-1 with specificity and have canine antibody kappa light chains comprising one to six different CDRs comprising at least 80%, 85%, 90%, 95%, 98% or 99% sequence identity with the amino acid sequences of SEQ ID NOs: 13, 14, 15, 16, 17, 18, 19, 20, 21, 22, 23, 24, 25, and/or 26 and canine antibody heavy chain IgG comprising one to six different CDRs comprising at least 80%, 85%, 90%, 95%, 98% or 99% sequence identity with the amino acid sequences of SEQ ID NOs: 27, 28, 29, 30, 31, 32, 33, 34, 35, 36, 37, 38, and/or 146, while still exhibiting the desired binding and functional properties. In another embodiment the antibody or antigen binding fragment of the present invention comprises a canine frame comprising of a combination of IgG heavy chain sequence with a kappa light chain having one or more of the above-mentioned CDR amino acid sequences with 0, 1, 2, 3, 4, or 5 conservative or non-conservative amino acid substitutions, while still exhibiting the desired binding and functional properties.

Sequence identity refers to the degree to which the amino acids of two polypeptides are the same at equivalent positions when the two sequences are optimally aligned. As used herein one amino acid sequence is 100% “identical” to a second amino acid sequence when the amino acid residues of both sequences are identical. Accordingly, an amino acid sequence is 50% “identical” to a second amino acid sequence when 50% of the amino acid residues of the two amino acid sequences are identical. The sequence comparison is performed over a contiguous block of amino acid residues comprised by a given protein, e.g., a protein, or a portion of the polypeptide being compared. In a particular embodiment, selected deletions or insertions that could otherwise alter the correspondence between the two amino acid sequences are taken into account.

Sequence similarity includes identical residues and nonidentical, biochemically related amino acids. Biochemically related amino acids that share similar properties and may be interchangeable are discussed

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity [see, e.g., Watson et al., Molecular Biology of the Gene, The Benjamin/Cummings Pub. Co., p. 224 (4th Ed.; 1987)]. In addition, substitutions of structurally or functionally similar amino acids are less likely to disrupt biological activity. Exemplary conservative substitutions are set forth in Table I directly below.

TABLE 1
EXEMPLARY CONSERVATIVE AMINO ACID SUBSTITUTIONS

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala; Gly |
| Ser (S) | Thr |
| Thr (T) | Ser |
| Trp (W) | Tyr; Phe |
| Tyr (Y) | Trp; Phe |
| Val (V) | Ile; Leu |

Function-conservative variants of the antibodies of the invention are also contemplated by the present invention. “Function-conservative variants,” as used herein, refers to antibodies or fragments in which one or more amino acid residues have been changed without altering a desired property, such an antigen affinity and/or specificity. Such variants include, but are not limited to, replacement of an amino acid with one having similar properties, such as the conservative amino acid substitutions of Table I above.

### Nucleic Acids,

The present invention further comprises the nucleic acids encoding the immunoglobulin chains of caninized murine anti-canine PD-1 antibodies and antigen binding fragments thereof disclosed herein (see Examples below).

Also included in the present invention are nucleic acids that encode immunoglobulin polypeptides comprising amino acid sequences that are at least about 70% identical, preferably at least about 80% identical, more preferably at least about 90% identical and most preferably at least about 95% identical (e.g., 95%, 96%, 97%, 98%, 99%, 100%) to the amino acid sequences of the CDRs and/or canine cFc's and/or antibodies provided herein when the comparison is performed by a BLAST algorithm wherein the parameters of the algorithm are selected to give the largest match between the respective sequences over the entire length of the respective reference sequences. The present invention further provides nucleic acids that encode immunoglobulin polypeptides comprising amino acid sequences that are at least about 70% similar, preferably at least about 80% similar, more preferably at least about 90% similar and most preferably at least about 95% similar (e.g., 95%, 96%, 97%, 98%, 99%, 100%) to any of the reference amino acid sequences when the comparison is performed with a BLAST algorithm, wherein the parameters of the algorithm are selected to give the largest match between the respective sequences over the entire length of the respective reference sequences.

As used herein, nucleotide and amino acid sequence percent identity can be determined using C, MacVector (MacVector, Inc. Cary, NC 27519), Vector NTI (Informax, Inc. MD), Oxford Molecular Group PLC (1996) and the Clustal W algorithm with the alignment default parameters, and default parameters for identity. These commercially available programs can also be used to determine sequence similarity using the same or analogous default parameters. Alternatively, an Advanced Blast search under the default filter conditions can be used, e.g., using the GCG (Genetics Computer Group, Program Manual for the GCG Package, Version 7, Madison, Wisconsin) pileup program using the default parameters.

The following references relate to BLAST algorithms often used for sequence analysis: BLAST ALGORITHMS: Altschul, S. F., et al., J. Mol. Biol. 215:403-410 (1990); Gish, W., et al., Nature Genet. 3:266-272 (1993); Madden, T. L., et al., Meth. Enzymol. 266:131-141 (1996); Altschul, S. F., et al., Nucleic Acids Res. 25:3389-3402 (1997); Zhang, J., et al., Genome Res. 7:649-656 (1997); Wootton, J. C., et al., Comput. Chem. 17:149-163 (1993); Hancock, J. M. et al., Comput. Appl. Biosci. 10:67-70 (1994); ALIGNMENT SCORING SYSTEMS: Dayhoff, M. O., et al., “A model of evolutionary change in proteins.” in Atlas of Protein Sequence and Structure, vol. 5, suppl. 3. M. O. Dayhoff (ed.), pp. 345-352, (1978); Natl. Biomed. Res. Found., Washington, DC; Schwartz, R. M., et al., “Matrices for detecting distant relationships.” in Atlas of Protein Sequence and Structure, vol. 5, suppl. 3.” (1978), M. O. Dayhoff (ed.), pp. 353-358 (1978), Natl. Biomed. Res. Found., Washington, DC; Altschul, S. F., J. Mol. Biol. 219:555-565 (1991); States, D. J., et al., Methods 3:66-70 (1991); Henikoff, S., et al., Proc. Natl. Acad. Sci. USA 89:10915-10919 (1992); Altschul, S. F., et al., J. Mol. Evol. 36:290-300 (1993); ALIGNMENT STATISTICS: Karlin, S., et al., Proc. Natl. Acad. Sci. USA 87:2264-2268 (1990); Karlin, S., et al., Proc. Natl. Acad. Sci. USA 90:5873-5877 (1993); Dembo, A., et al., Ann. Prob. 22:2022-2039 (1994); and Altschul, S. F. “Evaluating the statistical significance of multiple distinct local alignments.” in Theoretical and Computational Methods in Genome Research (S. Suhai, ed.), pp. 1-14, Plenum, New York (1997).

This present invention also provides expression vectors comprising the nucleic acids (including isolated nucleic acids) of the invention, wherein the nucleic acid is operably linked to control sequences that are recognized by a host cell when the host cell is transfected with the vector. Also provided are host cells comprising an expression vector of the present invention and methods for producing the antibody or antigen binding fragment thereof disclosed herein comprising culturing a host cell harboring an expression vector encoding the antibody or antigen binding fragment in culture medium, and isolating the antigen or antigen binding fragment thereof from the host cell or culture medium.

A caninized murine anti-canine PD-1 antibody for example, can be produced recombinantly by methods that are known in the field. Mammalian cell lines available as hosts for expression of the antibodies or fragments disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse, and hamster cells. Cell lines of particular preference are selected through determining which cell lines have high expression levels. Other cell lines that may be used are insect cell lines, such as Sf9 cells, amphibian cells, bacterial cells, plant cells and fungal cells. When recombinant expression vectors encoding the heavy chain or antigen-binding portion or fragment thereof, the light chain and/or antigen-binding fragment thereof are introduced into mammalian host cells, the antibodies are produced by culturing the host cells for a period of time sufficient to allow for expression of the antibody in the host cells or, more preferably, secretion of the antibody into the culture medium in which the host cells are grown.

Antibodies can be recovered from the culture medium using standard protein purification methods. Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

In general, glycoproteins produced in a particular cell line or transgenic animal will have a glycosylation pattern that is characteristic for glycoproteins produced in the cell line or transgenic animal. Therefore, the particular glycosylation pattern of an antibody will depend on the particular cell line or transgenic animal used to produce the antibody. However, all antibodies encoded by the nucleic acid molecules provided herein, or comprising the amino acid sequences provided herein, comprise the instant invention, independent of the glycosylation pattern that the antibodies may have. Similarly, in particular embodiments, antibodies with a glycosylation pattern comprising only non-fucosylated N-glycans may be advantageous, because these antibodies have been shown to typically exhibit more potent efficacy than their fucosylated counterparts both in vitro and in vivo [See for example, Shinkawa et al., J. Biol. Chem. 278:3466-3473 (2003); U.S. Pat. Nos. 6,946,292 and 7,214,775].

The present invention further includes antibody fragments of the caninized murine anti-canine PD-1 antibodies disclosed herein. The antibody fragments include F (ab) 2 fragments, which may be produced by enzymatic cleavage of an IgG by, for example, pepsin. Fab fragments may be produced by, for example, reduction of F(ab)₂ with dithiothreitol or mercaptoethylamine. A Fab fragment is a V_{L}-C_{L} chain appended to a V_{H}-C_{H1} chain by a disulfide bridge. A F(ab)₂ fragment is two Fab fragments which, in turn, are appended by two disulfide bridges. The Fab portion of an F(ab)₂ molecule includes a portion of the F_{c} region between which disulfide bridges are located. An Fv fragment is a V_{L} or V_{H} region.

In one embodiment, the antibody or antigen binding fragment comprises a heavy chain constant region, e.g., a canine constant region, such as IgGA, IgGB, IgGC and IgGD canine heavy chain constant region or a variant thereof. In another embodiment, the antibody or antigen binding fragment comprises a light chain constant region, e.g., a canine light chain constant region, such as lambda or kappa canine light chain region or variant thereof. By way of example, and not limitation the canine heavy chain constant region can be from IgGB and the canine light chain constant region can be from kappa.

### Antibody Engineering

Caninized murine anti-canine PD-1 antibodies of the present invention can be engineered to include modifications in the canine frame of a parental (i.e., canine) monoclonal antibody, e.g. to improve the properties of the antibody, as detailed below.

The cross-blocking caninized antibodies and antigen-binding fragments thereof discussed herein can be identified based on their ability to cross-compete with any of IB5, 3B6, 4D12, 7C9, 2H9, 5G5, and/or 2G9 in standard binding assays (e.g., BIACore®, ELISA, as exemplified below, or flow cytometry). For example, standard ELISA assays can be used in which a recombinant canine PD-1 protein is immobilized on the plate, one of the antibodies is fluorescently labeled and the ability of non-labeled antibodies to compete off the binding of the labeled antibody is evaluated. Additionally or alternatively, BIAcore® analysis can be used to assess the ability of the antibodies to cross-compete. The ability of a test antibody to inhibit the binding of, for example, IB5, 3B6, 4D12, 7C9, 2H9, 5G5, and/or 2G9, to canine PD-1 demonstrates that the test antibody can compete with IB5, 3B6, 4D12, 7C9, 2H9, 5G5, and/or 2G9 for binding to canine PD-1 and thus, may, in some cases, bind to the same epitope on canine PD-1 as IB5, 3B6, 4D12, 7C9, 2H9, 5G5, and/or 2G9. As stated above, antibodies and fragments that bind to the same epitope as any of the anti-canine PD-1 antibodies or fragments of the present invention also form part of the present invention.

### Pharmaceutical Compositions and Administration

To prepare pharmaceutical or sterile compositions of a caninized murine anti-canine PD-1 antibody or antigen binding fragment thereof it can be admixed with a pharmaceutically acceptable carrier or excipient. [See, e.g., Remington's Pharmaceutical Sciences and U.S. Pharmacopeia: National Formulary, Mack Publishing Company, Easton, PA (1984)].

Formulations of therapeutic and diagnostic agents may be prepared by mixing with acceptable carriers, excipients, or stabilizers in the form of, e.g., lyophilized powders, slurries, aqueous solutions or suspensions [see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY]. In one embodiment, anti-PD-1 antibodies of the present invention are diluted to an appropriate concentration in a sodium acetate solution pH 5-6, and NaCl or sucrose is added for tonicity. Additional agents, such as polysorbate 20 or polysorbate 80, may be added to enhance stability.

Toxicity and therapeutic efficacy of the antibody compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in canines. The dosage of such compounds lies preferably within a range of circulating concentrations that include the ED₅₀ with little or no toxicity. The dosage may vary within this range depending upon the dosage form employed and the route of administration.

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In particular embodiments, the caninized murine anti-canine PD-1 antibody or antigen binding fragment thereof can be administered by an invasive route such as by injection. In further embodiments of the invention, a murine anti-canine PD-1 antibody or antigen binding fragment thereof, or pharmaceutical composition thereof, is administered intravenously, subcutaneously, intramuscularly, intraarterially, intratumorally, or by inhalation, aerosol delivery. Administration by non-invasive routes (e.g., orally; for example, in a pill, capsule or tablet) is also within the scope of the present invention.

The pharmaceutical compositions disclosed herein may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternately, one may administer a caninized murine anti-canine PD-1 antibody in a local rather than systemic manner, for example, via injection of the antibody directly into an arthritic joint or pathogen-induced lesion characterized by immunopathology, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

The administration regimen depends on several factors, including the serum or tissue turnover rate of the therapeutic antibody, the level of symptoms, the immunogenicity of the therapeutic antibody, and the accessibility of the target cells in the biological matrix. Preferably, the administration regimen delivers sufficient therapeutic antibody to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies is available [see, e.g., Wawrzynczak Antibody Therapy, Bios Scientific Pub. Ltd, Oxfordshire, U K (1996); Kresina (ed.) Monoclonal Antibodies, Cytokines and Arthritis, Marcel Dekker, New York, NY (1991); Bach (ed.) Monoclonal Antibodies and Peptide Therapy in Autoimmune Diseases, Marcel Dekker, New York, NY (1993); Baert, et al. New Engl. J. Med. 348:601-608 (2003); Milgrom et al. New Engl. J. Med. 341:1966-1973 (1999); Slamon et al. New Engl. J. Med. 344:783-792 (2001); Beniaminovitz et al. New Engl. J. Med. 342:613-619 (2000); Ghosh et al. New Engl. J. Med. 348:24-32 (2003); Lipsky et al. New Engl. J. Med. 343:1594-1602 (2000)].

Determination of the appropriate dose is made by the veteranarian, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Antibodies or antigen binding fragments thereof disclosed herein may be provided by continuous infusion, or by doses administered, e.g., daily, 1-7 times per week, weekly, biweekly, monthly, bimonthly, quarterly, semiannually, annually etc. Doses may be provided, e.g., intravenously, subcutaneously, topically, orally, nasally, rectally, intramuscular, intracerebrally, intraspinally, or by inhalation. A total weekly dose is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more [see, e.g., Yang, et al. New Engl. J. Med. 349:427-434 (2003); Herold, et al. New Engl. J. Med. 346:1692-1698 (2002); Liu, et al. J. Neurol. Neurosurg. Psych. 67:451-456 (1999); Portielji, et al. Cancer Immunol. Immunother. 52:133-144 (2003)]. Doses may also be provided to achieve a pre-determined target concentration of a caninized murine anti-canine PD-1 antibody in the subject's serum, such as 0.1, 0.3, 1, 3, 10, 30, 100, 300 μg/ml or more. In other embodiments, a caninized murine anti-canine PD-1 antibody of the present invention is administered subcutaneously or intravenously, on a weekly, biweekly, “every 4 weeks,” monthly, bimonthly, or quarterly basis at 10, 20, 50, 80, 100, 200, 500, 1000 or 2500 mg/subject.

As used herein, “inhibit” or “treat” or “treatment” includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

As used herein, the terms “therapeutically effective amount”, “therapeutically effective dose” and “effective amount” refer to an amount of a caninized murine anti-canine PD-1 antibody or antigen binding fragment thereof of the present invention that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the binding compound sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity.

### Other Combination Therapies

As previously described, a caninized murine anti-canine PD-1 antibody or antigen binding fragment thereof may be coadministered with one or other more therapeutic agents (such as a chemotherapeutic agent). The antibody may be linked to the agent (as an immunocomplex) or can be administered separately from the agent. In the latter case (separate administration), the antibody can be administered before, after or concurrently with the agent or can be coadministered with other known therapies.

### Kits

Further provided are kits comprising one or more components that include, but are not limited to, an antibody or antigen binding fragment, as discussed herein, which specifically binds PD-1 (e.g., a caninized murine anti-canine PD-1 antibody or antigen binding fragment thereof) in association with one or more additional components including, but not limited to a pharmaceutically acceptable carrier and/or a chemotherapeutic agent, as discussed herein. The binding composition and/or the chemotherapeutic agent can be formulated as a pure composition or in combination with a pharmaceutically acceptable carrier, in a pharmaceutical composition.

In one embodiment, the kit includes a binding composition of the present invention (e.g., a caninized murine anti-canine PD-1 or a pharmaceutical composition thereof in one container (e.g., in a sterile glass or plastic vial) and a pharmaceutical composition thereof and/or a chemotherapeutic agent in another container (e.g., in a sterile glass or plastic vial).

If the kit includes a pharmaceutical composition for parenteral administration to a subject, the kit can also include a device for performing such administration. For example, the kit can include one or more hypodermic needles or other injection devices as discussed above. The kit can also include a package insert including information concerning the pharmaceutical compositions and dosage forms in the kit. Generally, such information aids pet owners and veteranarians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

As a matter of convenience, an antibody or specific binding agent disclosed herein can be provided in a kit, i.e., a packaged combination of reagents in predetermined amounts with instructions for performing the diagnostic or detection assay. Where the antibody is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

### EXAMPLES

### Example 1

### Canine Pd-1 and Pd-L1

### Canine PD-1 and PD-L1:

U.S. provisional application No. 61/918,946, filed on Dec. 20, 2013, hereby incorporated by reference in its entireties, provides: the full length nucleotide sequence for canine PD-1 (cPD-1) of SEQ ID NO: 113 [SEQ ID NO: 133 includes the signal sequence]; the corresponding translated amino acid sequence of SEQ ID NO: 114 [SEQ ID NO: 134 includes the signal sequence]; the nucleotide sequence encoding the extra-cellular domain (ECD) of canine PD-1, SEQ ID NO: 115; the amino acid sequence of the ECD of canine PD-1, SEQ ID NO: 116; the nucleotide sequence of canine PD-1 ECD plus a GT linker and the Fc part of human IgG1 Fc gene, of SEQ ID NO: 117; and the amino acid sequence of the canine PD-1 ECD plus a GT linker and the Fc part of human IgG1 Fc gene, SEQ ID NO: 118 [SEQ ID NO: 137 includes the signal sequence].

U.S. provisional application No. 61/918,946 further provides: the full length nucleotide sequence for canine PD-L1 (cPD-L1) of SEQ ID NO: 119 [SEQ ID NO: 135 includes the signal sequence]; the corresponding translated amino acid sequence of SEQ ID NO: 120 [SEQ ID NO: 136 includes the signal sequence]; the nucleotide sequence encoding the extra-cellular domain (ECD) of canine PD-L1, SEQ ID NO: 121; the amino acid sequence of the ECD of canine PD-L1, SEQ ID NO: 122; the nucleotide sequence of canine PD-L1 ECD plus a GT linker and the Fc part of human IgG1 Fc gene, SEQ ID NO: 123; and the amino acid sequence of canine PD-L1 ECD plus a GT linker and the Fc part of human IgG1 Fc gene, SEQ ID NO: 124.

### Example 2

### Murine Anti-Canine Pd-1 Antibodies

### Generation of Anti-Canine PD1 Monoclonal Antibodies:

A total of three Balb/c mice were immunized multiple times (with 10 μg each time) over a 17 day period. The immunizing antigen was the canine PD-1 ECD-Fc fusion protein. Following immunization, serum was collected from each mouse and tested for reactivity with canine PD-1 ECD-HIS tagged protein. The spleen cells of the mouse with the highest serum anti-PD-1 ECD-HIS titer were fused to the myeloma P3×63Ag8.653 cell line. Approximately 2 weeks following fusion, supernatant from putative hybridoma cells were tested by ELISA for their reactivity to the PD-1 ECD-HIS tagged protein. Hybridomas producing strong positive signals in the ELISA were subcloned by limiting dilution and tested again for reactivity to canine PD-1 ECD-HIS tagged protein.

### Confirmation of Monoclonal Antibodies Reactivity Against Canine PD-1:

The reactivity of antibodies secreted by hybridomas to ECD of canine PD-1 was confirmed by ELISA. Hybridoma cells were cultured using CELLine bioreactors (Integra-biosciences) for 10-30 days. Cells were initially maintained in DMEM supplemented with 4 mM L-glutamine and 10% Ultra Low IgG fetal bovine serum (FBS) from Gibco. Hybridoma cells were seeded in CELLine bioreactor cell chambers at a cell density of approximately 2×10⁶ cells/mL in 15 mL of the same medium with the FBS concentration increased to 20%. The outer chamber was filled with 1 L of nutrient medium (DMEM with 4 mM L-glutamine and 2% standard FBS). Hybridoma cells in the cell chamber were expanded to approximately 2.5×10⁷ cells/mL over 3-7 days. Then, 10 mL of cell suspension was harvested from the cell chamber and replaced with fresh media to allow for re-expansion of cells and subsequent harvests. This procedure was repeated as necessary to obtain adequate amounts of mAb from each hybridoma clone. Harvested cell suspensions were centrifuged and the supernatants were filtered through 0.2 micron filter membranes. For antibody purification, each clone's supernatant was purified using a Protein G Sepharose 4 Fast flow 5 mL column (GE Healthcare) by gravity flow. After washing with Tris-EDTA (TE) buffer pH 8.0, bound antibodies were eluted using 0.1 M glycine buffer, pH 2.7, followed by pH neutralization using 1 M Tris, pH 8.0. Antibodies were concentrated and buffer exchanged into phosphate-buffered saline (PBS) using Centriprep® YM-10.10 kDa NMWL centrifugal filter units (Millipore). Antibody concentrations were quantified using spectrophotometry.

Purified anti-canine PD-1 mAbs were tested for reactivity with the HIS-tagged ECD domain of canine PD-1 by ELISA as follows: HIS-tagged canine PD-1 ECD protein is diluted to 10 μg/mL in coating buffer (Carbonate/Bicarbonate pH 9.0) and dispensed at 100 μl/well in 96-well flat bottomed ELISA plates (NUNC). The plates are incubated at 4° C. overnight. The plates are then washed three times with phosphate buffered saline containing 0.05% Tween® 20 (PBST). Next, 200 μl of blocking buffer (5% skim milk in PBST) is added to each well and the plates are incubated at 37° C. for 60 minutes. The plates are then washed three times with PBST. Next, 100 μl of test mAbs diluted in blocking buffer is added to the first wells of the appropriate columns. Test mAbs are then diluted two-fold to the appropriate plate position. Following incubation of the plates at 37° C. for 60 minutes, the plates are washed three times with PBST. Next, 100 μl per well of a 1:2,000 dilution of a horseradish peroxidase conjugated goat anti-mouse IgG (KPL) is added to the plates, which are then incubated at 37° C. for 60 minutes. Then the plates are washed three times with PBST, and 100 μl/well of 3,3′,5,5′ tetramethyl benzidine, (TMB) substrate (from KPL) is added to the plates. The color reaction is allowed to develop for 5-20 minutes at 37° C. prior to measuring absorbance at 650 nm.

### Cho Cells Expressing Canine PD-1 Protein:

The full length canine PD-1 gene was cloned into plasmid p96793. In this plasmid the expression of the PD-1 protein is driven by an hCMV promoter. CHO DXB11 cells (dhfr-) were maintained in MEM-alpha (Gibco) supplemented with 10% fetal bovine serum. Transfection of CHO cells with plasmid p96793 was carried out in 75 cm² flasks containing approximately 6×10⁶ cells by liposome-mediated gene delivery using Lipofectamine (Invitrogen). After 48 hours, cells were passaged into MEM-alpha medium without nucleosides, supplemented with 10% FBS and 400 μg/mL hygromycin B (selective medium). Limited-dilution cloning was performed on the pool of dhfr+, hygromycin resistant cells. Clones were assessed for expression of canine PD-1 by immunofluorescence assay. Briefly, cell monolayers were fixed in 96 well plates with 80% acetone. Fixed and dried cell monolayers were then incubated for 1 hour with a polyclonal goat anti-human PD-1 antibody (R&D Systems). Plates were washed with PBS, then incubated for 1 hour with a fluorescein-labeled rabbit anti-goat IgG antibody (KPL). Plates were washed with PBS. Clones exhibiting fluorescence were expanded and cell stocks were established.

Reactivity of Mouse mAbs Against Canine PD-1 Proteins Expressed on CHO Cells:

The reactivity of mouse anti-canine PD-1 mAbs with canine PD-1 on CHO cells was determined by a cell-based assay using CHO cells that express PD-1. Briefly, the CHO cells expressing canine PD-1 were cultured to 80-100% confluency in 50 μl media (DMEM/HAM's F12, 10% FBS). Next, 50 μl of media containing various concentrations of purified mAbs were added for 1 hour at 37° C. Following three washes with PBS-Tween, 100 μl of goat anti-mouse horse raddish peroxidase (HRP) diluted 1:1000 in culture media was added for one hour at 37° C. After three additional washes with PBS-Tween, bound mAbs were visualized with a perioxidase substrate (TMB). The absorbance increase due to perioxidase activity at 450 nm was measured in a microplate reader.

### Characterization of Mouse Anti-Canine PD-1 Antibodies:

As detailed above, as well as in U.S. provisional application No. 61/918,946, filed on Dec. 20, 2013, hereby incorporated by reference in its entireties, the mouse anti-canine PD-1 antibodies were characterized by a number of parameters including: their reactivity with the ECD of canine PD-1 by ELISA, their reactivity with PD-1 expressed on the surface of CHO cells, their ability to block the binding of PD-1 with it PD-L1, and their ability to bind to PBMC cells from healthy dogs and dogs with cancer. The amino acid sequences of the CDRs of the seven mouse anti-canine PD-1 antibodies selected (denoted as IB5, 2G9, 2H9, 3B6, 4D12, 5G5, and 7C9, respectively) had substantial homology as demonstrated in Table 2 below.

TABLE 2
AMINO ACID SEQUENCES OF THE CDRs

SEQ
ID NO.

VL CDR1

| | | |
|---|---|---|
| 1B5 | Lys Ser Ser Gln Ser Leu Leu Asn | 13 |
| | Ser Val Asn Gln Lys Asn Tyr Leu | |
| | Ala | |
| 2G9 | Arg Ser Ser Gln Asn Ile Val His | 14 |
| | Ser Asn Gly Asn Thr Tyr Leu Glu | |
| 2H9 | His Ala Ser Gln Asn Ile Asn Val | 15 |
| | Trp Leu Ser | |
| 3B6 | Lys Ser Ser Gln Ser Leu Leu Asn | 13 |
| | Ser Val Asn Gln Lys Asn Tyr Leu | |
| | Ala | |
| 4D12 | Lys Ser Ser Gln Ser Leu Leu Asn | 13 |
| | Ser Val Asn Gln Lys Asn Tyr Leu | |
| | Ala | |
| 5G5 | His Ala Ser Gln Asn Ile Asn Val | 15 |
| | Trp Leu Ser | |
| 7C9 | Lys Ser Ser Gln Ser Leu Leu Asn | 13 |
| | Ser Val Asn Gln Lys Asn Tyr Leu | |
| | Ala | |

VL CDR2

| | | |
|---|---|---|
| 1B5 | Phe Ala Ser Thr Arg Val Ser | 16 |
| 2G9 | Lys Val Ser Asn Arg Phe Ser | 17 |
| 2H9 | Lys Ala Ser His Leu His Thr | 18 |
| 3B6 | Phe Ala Ser Ala Arg Val Ser | 19 |
| 4D12 | Phe Ala Ser Thr Arg Ile Ser | 20 |
| 5G5 | Lys Ala Ser Asn Leu His Thr | 21 |
| 7C9 | Phe Ala Ser Thr Arg Val Ser | 16 |

VL CDR3

| | | |
|---|---|---|
| 1B5 | Gln Gln Tyr Phe Ser Thr Pro Leu | 22 |
| | Thr | |
| 2G9 | Phe Gln Gly Ser His Val Pro Tyr | 23 |
| | Thr | |
| 2H9 | Gln Gln Gly Gln Ser Trp Pro Leu | 24 |
| | Thr | |
| 3B6 | Gln Gln Tyr Phe Ser Thr Pro Leu | 25 |
| | Thr | |
| 4D12 | Gln Gln Tyr Phe Ser Thr Pro Leu | 25 |
| | Thr | |
| 5G5 | Gln Gln Gly Gln Ser Tyr Pro Leu | 26 |
| | Thr | |
| 7C9 | Gln Gln Tyr Phe Ser Thr Pro Leu | 22 |
| | Thr | |

VH CDR1

| | | |
|---|---|---|
| 1B5 | Gly Tyr Thr Phe Thr Thr Tyr Gly | 27 |
| | Met Ser | |
| 2G9 | Gly Tyr Thr Phe Thr Arg Tyr Asn | 28 |
| | Met His | |
| 2H9 | Gly Phe Asn Ile Lys Asn Thr Tyr | 29 |
| | Met His | |
| 3B6 | Gly Tyr Thr Phe Thr Thr Tyr Gly | 27 |
| | Met Ser | |
| 4D12 | Gly Tyr Thr Phe Thr Thr Tyr Gly | 27 |
| | Met Ser | |
| 5G5 | Gly Phe Asn Ile Lys Asn Thr Tyr | 29 |
| | Met His | |
| 7C9 | Gly Phe Ser Leu Thr Ser Tyr Gly | 30 |
| | Val His | |

VH CDR2

| | | |
|---|---|---|
| 1B5 | Trp Ile Asn Ile Tyr Ser Gly Ile | 31 |
| | Pro Thr Tyr Ala Asp Asp Phe Lys | |
| | Gly | |
| 2G9 | Thr Ile Tyr Pro Gly Tyr Gly Asp | 32 |
| | Thr Ser Tyr Asn Gln Lys Phe Lys | |
| | Gly | |
| 2H9 | Arg Ile Ala Pro Ala Asn Val Asp | 33 |
| | Thr Lys Tyr Ala Pro Lys Phe Gln | |
| | Gly | |
| 3B6 | Trp Ile Asn Ile Tyr Ser Gly Ile | 31 |
| | Pro Thr Tyr Ala Asp Asp Phe Lys | |
| | Gly | |
| 4D12 | Trp Ile Asn Ile Tyr Ser Gly Met | 34 |
| | Pro Thr Tyr Ala Asp Asp Phe Lys | |
| | Gly | |
| 5G5 | Arg Ile Asp Pro Ala Asn Val Asn | 35 |
| | Thr Lys Tyr Ala Pro Lys Phe Gln | |
| | Gly | |
| 7C9 | Trp Ile Asn Ile Tyr Ser Gly Ile | 31 |
| | Pro Thr Tyr Ala Asp Asp Phe Lys | |
| | Gly | |

VHCDR3

| | | |
|---|---|---|
| 1B5 | Phe Asp Gly Pro Asp Tyr | 36 |
| 2G9 | Glu Phe Ala Asp Asp Tyr Pro Ile | 37 |
| | Pro Pro Phe Asp Tyr | |
| 2H9 | Ile Tyr Tyr Asp Tyr Asp Gly Asp | 38 |
| | Ile Asp Val | |
| 3B6 | Phe Asp Gly Pro Asp Tyr | 36 |
| 4D12 | Phe Asp Gly Pro Asp Tyr | 36 |
| 5G5 | Ile Phe Tyr Asp Tyr Asp Gly Asp | 146 |
| | Ile Asp Val | |
| 7C9 | Phe Asp Gly Pro Asp Tyr | 36 |

| | |
|---|---|
| mAbs: 4D12, 3B6, 7C9, | CDR: H1-1; CDR2: H2-1; CDR3: H3-6 |
| and 1B5: | |
| mAb: 5G5: | CDR: H1-1; CDR2: H2-1; CDR3: H3-11 |
| mAb: 2H9 | CDR: H1-1; CDR2: H2-2A; CDR3: H3-11 |
| mAb: 2G9 | CDR: H1-1; CDR2: H2-2A; CDR3: H3-13 |

| | |
|---|---|
| mAbs: 4D12, 3B6, | CDRL: L1-3; CDR2: L2-1; CDR3: L3-1 |
| 7C9, 1B5: | |
| mAb: 5G5: | CDR: L1-2A; CDR2: L2-1; CDR3: L3-1 |
| mAb: 2H9 | CDR: L1-2A; CDR2: L2-1; CDR3: L3-1 |
| mAb: 2G9 | CDR: L1-4; CDR2: L2-1; CDR3: L3-1 |

### Example 3

### Caninization and Characterization of Caninized Antibodies

In order to produce caninized antibodies it was necessary to identify the DNA sequence encoding the heavy and light chains of canine IgG. The nucleotide and amino acid sequences of the canine heavy chain can be obtained from the NCBI gene and protein databases. There are four known IgG subclasses of canine IgG: IgGA, IgGB, IgGC, and IgGD and two types of light chains: kappa and lambda. Table 7 lists the amino acid and nucleotide SEQ ID NOs of the unmodified canine Fc fragments.

Without being bound by any specific approach, the process of producing variants of anti-PD-1 monoclonal antibodies with various contents of canine and mouse sequences involved the general following scheme:
i) Determine the nucleotide sequence of VH and VL chains of mouse mAbs;
ii) Identify the H and L chain CDRs of mouse mAbs;
iii) Identify a suitable H and L chain of canine IgG;
iv) Determine the nucleotide sequence of canine IgG H and L chains;
v) Replace the nucleotide sequence encoding endogenous canine H and L chain CDRs with nucleotide sequences encoding the respective mouse CDRs. Also, optionally replace some canine framework residues with selected residues from the mouse framework regions;
vi) Synthesize the nucleotide from step (v) and insert it into a suitable expression plasmid; Transfect plasmids into appropriate cells, e.g., HEK 293 cells;
vii) Purify the expressed antibody from HEK 293 supernatent; and
viii) Test purified antibody for binding to canine PD-1.

A set of experiments was conducted following the above steps which resulted in a set of variant caninized antibodies with various contents of canine and mouse sequences.

Reactivity of caninized mAbs against canine PD-1 proteins expressed on CHO cells: The reactivity of caninized anti-canine PD-1 mAbs with canine PD-1 on CHO cells was determined by a cell-based assay using CHO cells that express canine PD-1. Briefly, the CHO cells expressing canine PD-1 were cultured to 80-100% confluency in 50 μl media (DMEM/HAM's F12, 10% FBS). Next, 50 μl of media containing various concentrations of purified mAbs were added for 1 hour at 37° C. Following three washes with PBS-Tween, 100 μl of goat anti-dog horse raddish peroxidase (HRP)-labelled antibody diluted 1:1000 in culture media was added for one hour at 37° C. After three additional washes with PBS-Tween, bound mAbs were visualized with a perioxidase substrate (TMB). The absorbance increase due to peroxidase activity at 450 nm was measured in a microplate reader.

Binding Studies of Mouse Anti-Canine PD-1 mAbs and Caninized Mouse Anti-Canine PD-1 mAbs with Canine PD-1

Approximately 70 resonance units (RU) of the canine PD-1 antigen was immobilized directly by amine coupling. Affinity measuments were made via label-free surface plasmon resonance based technology (e.g., Biacore® T200) with an association time of 300 seconds, a dissociation time of 1200 seconds, and at concentrations of 50, 100, 200 (×2) 400, and 800 nanomolar (nM). A fitting model of 1:1 binding was used. The antigen (canine PD-1) was immobilized on the sensor chip through amine coupling and the four antibodies as indicated in Table 14 below, were used as analytes that flowed through the antigen surface. The results demonstrated that the binding affinities of the anti-canine PD-1 antibodies of the present invention for the canine PD-1 antigen were strong, having nanomolar and even subnanomolar dissociation constants (Kd). Moreover, the mouse anti-canine PD-1 monoclonal antibody and the corresponding caninized mouse anti-canine PD-1 monoclonal antibody from the same clone yielded strikingly similar Kd values (see Table 14 below).

TABLE 14
Binding Constant Deteminations

| | kₒₙ (k¹) | k_{off} (k⁻¹) | Kd | Chi² | Rmax |
|---|---|---|---|---|---|
| Antibody | M⁻¹s⁻¹ | s⁻¹ | M | (RU²) | (RU) |
| Murine 2H9 | 2.3 × 10⁻⁴ | ≤5 × 10^{-6#} | ≤2.0 × 10^{-10#} | 0.19 | 25.6 |
| Caninized 2H9 | 1.0 × 10⁻⁴ | 5.9 × 10⁻⁶ | 5.9 × 10⁻¹⁰ | 0.10 | 27.7 |
| Murine 3B6 | 1.8 × 10⁻⁴ | 3.4 × 10⁻⁵ | 2.0 × 10⁻⁹ | 0.13 | 48.7 |
| Caninized 3B6 | 1.6 × 10⁻⁴ | 4.7 × 10⁻⁵ | 2.9 × 10⁻⁹ | 0.07 | 49.9 |

^{#}The off-rate was so slow that it was below the detection limit of the instrument used.

Ligand Blockade by Caninized Anti-Canine PD1 mAbs:

For caninized antibodies which react with canine PD-1 (cPD-1), a cell-based ELISA (CELISA) assay was used which is based on the CHO cell line expressing canine PD-1. Briefly, cPD-1 CHO cells were placed in 96-well plates at 4×10⁴ cells per well and the cells were incubated at 37° C. for 18-24 hours till they are 95-100% confluent. The cell culture media was aspirated off, the plates were washed 3× with PBS plus 0.05% Tween® 20 and 1×CHO media. 3-fold serial dilutions of caninized anti-cPD1 mAbs were made in CHO media, starting at 30 μg/mL, and 50 μL/well of each antibody dilution was added down the plate. The plates were then incubated at 37° C., 5% CO₂ for 30 min, with shaking. Human PD-L1-Fc was added to 4 μg/ml in CHO media, 50 μL/well without removing or washing the incbuated anti-PD1 mAbs, then incubated at 37° C., 5% CO₂ for 45 min, with shaking. The plates were washed with 6× with PBS plus 0.05% Tween® 20. 100 μl/well of anti-human Fc-HRP (Calbiochem) (1:2500) in CHO media was added and incubated at 37° C./5% CO₂ for 30-60 min. (anti-human Fc-HRP does not bind canine Fc.) The plates were washed with 5×PBS plus 0.05% Tween® 20. 100 μl/well TMB microwell substrate was added and then incubated at room temp for 10 minutes. The reaction was stopped with 100 μl/well 1.5 M phosphoric acid. Measure A450-A620 on the ELISA reader.

Cytokine Release from Dog PBMC:

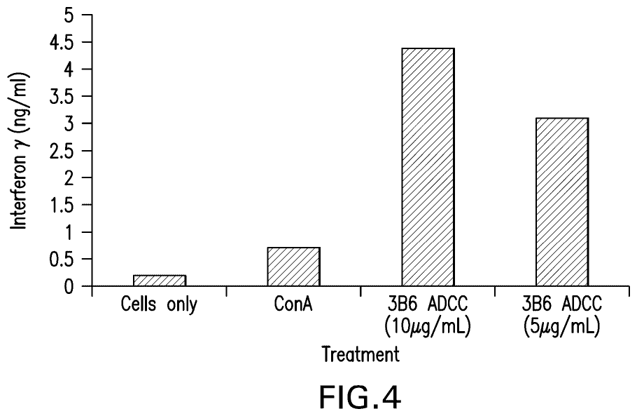

PBMC were prepared from EDTA blood samples obtained from healthy dogs and dogs with cancer, using Ficoll separation. Cells were washed 3 times, and resuspended in complete tissue culture medium at a concentration of 2.5×10⁵ cells per well in triplicate wells in 96-well plates. Cells were activated with concanavalin A at 1 μg/ml. Test antibodies were added at various concentrations and the cultures were incubated for 96 hours. Controls included cells incubated with conA and no antibody, or conA and irrelevant isotype-matched antibodies. After 96 hours in culture, supernatants were collected and assayed for IFN-_{γ} release, using a commercial canine IFN-_{γ} ELISA kit (R & D Systems) [see, FIG. 4].

### Example 4

### Genetically Modified Canine Iggs

In order to generate variants of canine IgG that lack effector functions, a number of mutant canine IgGB heavy chains were generated. These variants may include one of the following single or combined substitutions in the Fc portion of the heavy chain amino acid sequence: P4A, D31A, N63A, G64P, T65A, A93G, and P95A. Variant heavy chains (i.e., containing such amino acid substitutions) were cloned into expression plasmids and transfected into HEK 293 cells along with a plasmid containing the gene encoding a light chain. Intact antibodies expressed and purified from HEK 293 cells were evaluated for binding to Fc_{γ}RI and C1q to assess their potential for mediation of immune effector functions. Table 3 lists examples of the plasmids encoding the genetically modified caninized heavy chains, the caninized heavy chains; and the genetic modifications in these heavy chains. The variant heavy chains were used for assessment of effector function in the genetically modified mAbs. All of the heavy chains comprised the CDRs from the 2H9 murine anti-canine PD-1 antibody.

TABLE 3

| | | | AA position in |
|---|---|---|---|
| Plasmid | Heavy chain | Modification | native Fc |
| YZZ1057/ | can2H9VH4 | D31 to A | D31 |
| Mut-1 | | | |
| YZZ1058/ | can2H9VH4 | N63 to A | N63 |
| Mut-2 | | | |
| YZZ1062 | can2H9VH4 | D31 to A + N63 to A | D31 and N63 |
| YZZ1059 | can2H9VH4 | P4 to A | P4 |
| YZZ1060 | can2H9VH4 | A93 to G | A93 |
| YZZ1061 | can2H9VH4 | P95 to A | P95 |
| YZZ1068 | can2H9VH4 | D31 to A, N63 to | D31, N63, P4, |
| | | A, P4 to A, A93 to G, | A93, P95 |
| | | and P95 to A | |

### Fc_{γ}RI Binding:

Binding to FcR_{γ}I is a measure of the ability of an antibody to mediate ADCC. In order to assess this property for the caninized antibodies an assay to measure binding of caninized antibodies to Fc_{γ}RI was conducted as follows: Coat 96-well plates with 100 μl per well of 2.5 μg/mL PD-1 HIS. Incubate at 2-7° C. overnight. Equilibrate the plates to room temp for 15 minutes. Wash plates 3× with phosphate buffered saline containing 0.05% Tween® 20 (PBST) and then block the wells using 200 μL/well of 5% NFDM (Non Fat Dried Milk). Incubate for 60 minutes at 36-38° C. Wash 3× with PBST. Make 2-fold dilution of antibodies starting at 1 μg/mL in 5% NFDM. Add 100 μL/well of diluted antibodies. Incubate for 60 minutes at 36-38° C. Wash 6× with PBST. Add 100 μL/well of recombinant human CD64 protein (R&D systems) diluted to 1 μg/mL. Incubate for 60 minutes at 36-38° C. Wash 6× with PBST. Add 100 μL/well biotinylated-anti-CD64 antibody (R&D systems) diluted to 1:3000. Incubate for 60 minutes at 36-38° C. Wash 6× with PBST. Add 100 μL/well Streptavidin-HRP antibody (R&D systems) diluted to 1:7500. Incubate for 60 minutes at 36-38° C. Wash 6× with PBST. Add 100 μL/well TMB substrate. Incubate for 10 minutes at 15-30° C. Read plates using ELISA plate reader at 450-540 nm.

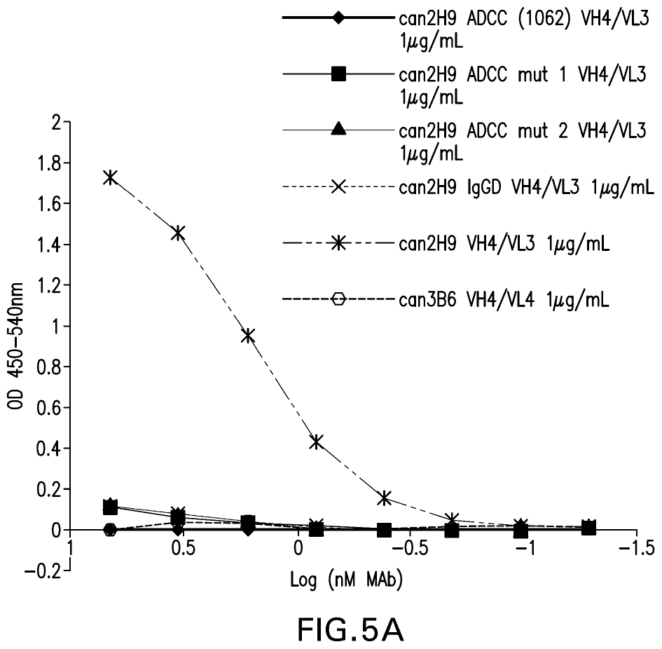

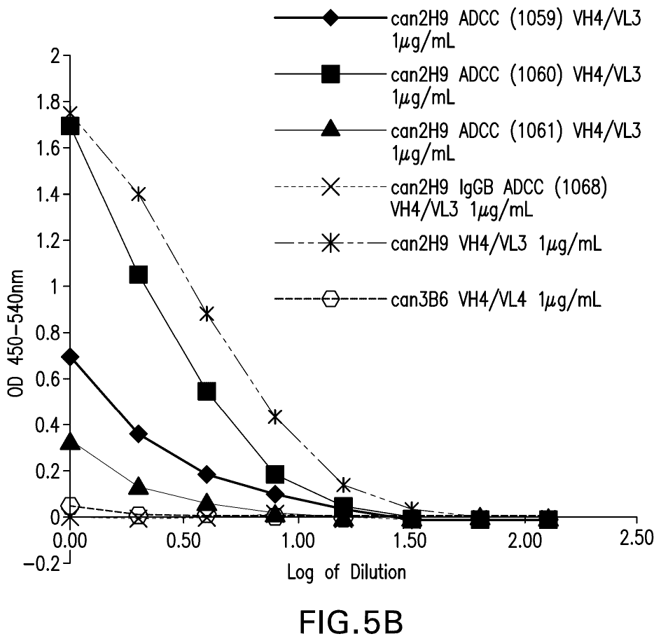

Results: FIG. 5A shows that caninized mAb designated can2H9 ADCC mut-1 VH4/VL3 which has the genetic modification of D31A or the mAb designated can2H9 ADCC mut-2 VH4/VL3 which has the genetic modification of N63A display a near complete reduction in binding to Fc_{γ}RI. On the other hand, the mab designated can2H9 ADCC (1062) VH4/VL3 which contains the combined D31A plus N63A genetic modifications lacks detectable binding to Fc_{γ}RI. In FIG. 5A, can2H9 IgGD VH4/VL3 is a caninized antibody which contains the Fc from canine IgGD and can3B6 VH4/VL4 IgGB is a caninized antibody that does not bind to the coating antigen (PD-1 HIS), and caninized mAb designated can2H9 VH4/VL3 is an antibody that contains un-modified IgGB Fc. FIG. 5B shows that the caninized mAb designated can2H9 ADCC (1059) VH4/VL3 which contains the genetic modification of P4A and the mAb designated can2H9 ADCC (1061) VH4/VL3 which contain the genetic modification of P95A display considerable reduction in binding to Fc_{γ}RI, whereas the mAb designated can2H9 ADCC (1060) VH4/VL3 which contains the genetic modification of A93G displays a slight reduction in binding to Fc_{γ}RI. On the other hand, the mAb designated can2H9 IgGB ADCC (1068) VH4/VL3 which contains five genetic modifications (D31A, N63A, P4A, A93G, P95A) is completely lacking in binding to Fc_{γ}RI.

### C1q Binding:

Binding to the first component of complement, C1q, is a measure of the ability of an antibody to mediate CDC. In order to assess this property for the caninized antibodies an assay to measure binding of caninized antibodies to C1q was conducted as follows: Coat 96-well plates with 2.5 μg/mL PD-1 HIS. Incubate at 2-7° C. overnight. Equilibrate to room temperature for 15 minutes. Wash with PBST 3×. Block with 200 μL/well with 5% BSA. Incubate for 60 minutes at 36-38° C. Wash with PBST 3×. Make a 2-fold dilution of antibodies starting at 1 μg/mL in 5% BSA. Add 100 μL/well diluted antibodies. Incubate for 60 minutes at 36-38° C. Wash with PBST 6×. Add 100 μL/well C1q protein diluted to 4 μg/mL. Incubate for 60 minutes at 36-38° C. Wash with PBST 6×. Add 100 μL/well Goat-Anti-C1q antibody diluted to 1:3000. Incubate for 60 minutes at 36-38° C. Wash with PBST 6×. Add 100 μL/well of donkey-anti-Goat-HRP antibody diluted 1:10000. Incubate for 60 minutes at 36-38° C. Wash with PBST 6×. Add 100 μL/well TMB substrate. Incubate for 10 minutes at 15-30° C. Read on ELISA plate reader at 450-540 nm.

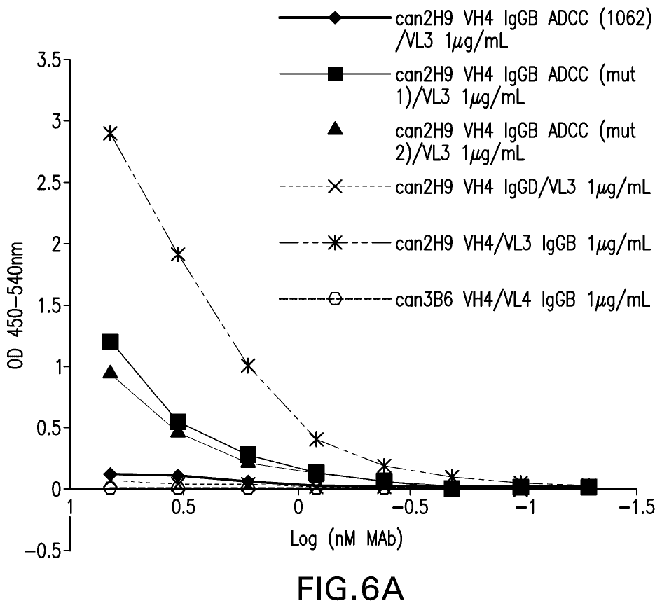

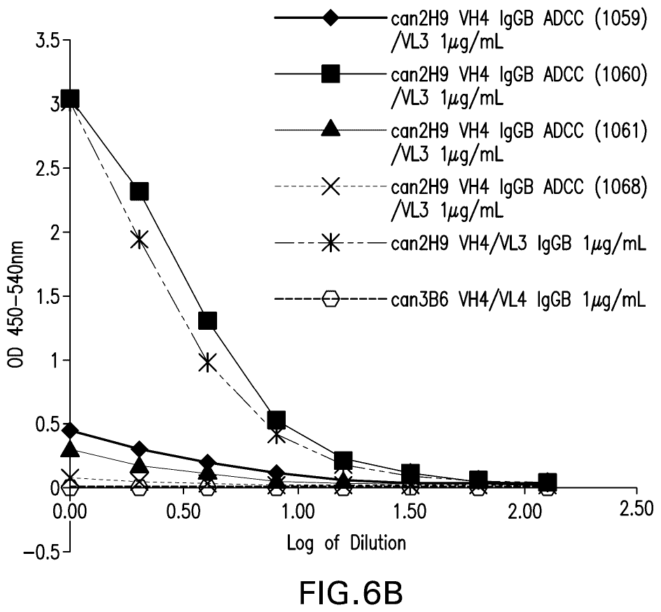

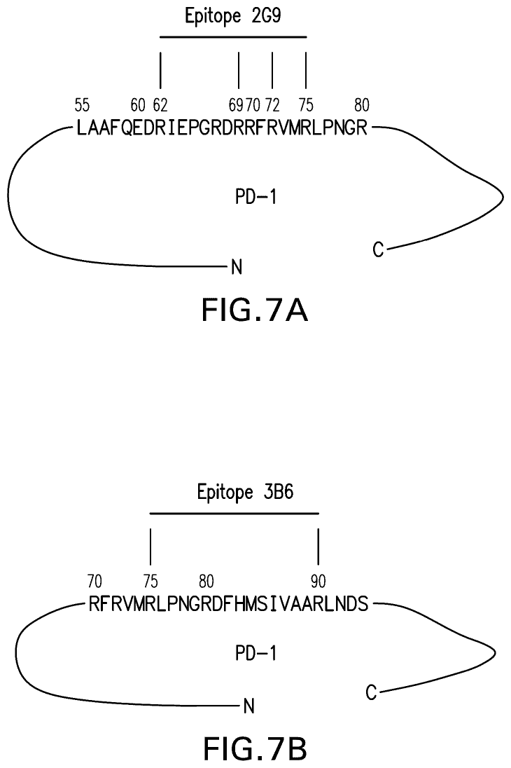

Results: FIG. 6A shows that the caninized mAb designated can2H9VH4 IgGB ADCC (mut-1)/VL3, which has the genetic modification of D31A or the mAb designated can2H9 VH4 IgGB ADCC (mut-2)/VL3 which has the genetic modification N63A display considerable reduction in binding to C1q. On the other hand, the mAb designated can2H9 VH4 IgGB ADCC (1062)/VL3 which contains the combined D31A plus N63A genetic modifications lacks detectable binding to C1q.

In FIG. 6A, can2H9 VH4 IgGD/VL3 is a caninized antibody which contains the Fc from canine IgGD and can3B6 VH4/VL4 IgGB is a caninized antibody that does not bind to the coating antigen (PD-1 HIS), and caninized mAb designated can2H9 VH4/VL3 IgGB is an antibody that contains un-mutated IgGB Fc. FIG. 6B shows that the caninized mAb designated can2H9 VH4 IgGB ADCC (1059)/VL3 which contains the substitution P4A and the mAb designated can2H9 VH4 IgGB ADCC (1061)/VL3 which contains the substitution P95A display considerable reduction in binding to C1q, whereas the mAb designated can2H9 VH4 IgGB ADCC (1060)/VL3 which contains the substitution A93G display an enhancement in binding to C1q. On the other hand, the mAb designated can2H9 VH4 IgGB ADCC (1068)/VL3 which contains five substitutions (D31A, N63A, P4A, A93G, P95A), is completely lacking in binding to C1q. In FIG. 6B, the mAb designated can3B6 VH4/VL4 IgGB is a caninized antibody that does not bind to the coating antigen (PD-1 HIS), and caninized mAb designated can2H9 VH4/VL3 IgGB is an antibody that contains un-mutated IgGB Fc.

TABLE 4
MODIFIED cFc or NATIVE cFc WITH HINGE SEQUENCES

| # | N. | A. | Modified Fs |
|---|---|---|---|
| 1* | ✓ | | Modified Fc-cIgGB |
| 2* | | ✓ | Modified Fc-cIgGB |
| 3* | ✓ | | Modified Fc-cIgGC |
| 4* | | ✓ | Modified Fc-cIgGC |
| 5^{#} | ✓ | | cIgGD Fc with S of cIgGD hinge to P |
| 6^{#} | | ✓ | cIgGD Fc with S of cIgGD hinge to P |
| 7 | ✓ | | cIgGD Fc with A hinge |
| 8 | | ✓ | cIgGD Fc with A hinge |
| 9 | ✓ | | cIgGD Fc with B hinge |
| 10 | | ✓ | cIgGD Fc with B hinge |
| 11 | ✓ | | cIgGD Fc with C hinge |
| 12 | | ✓ | cIgGD Fc with C hinge |

*The substitutions are at P4, D31, N63, G64, T65, A93, and P95 of amino acid sequences SEQ ID NOs: 2 and 4; or at the nucleotides that encode those amino acids for nucleotide sequences SEQ ID NOs: 1 and 3.
^{#}Single amino acid substitution as shown in Table 5 below in hinge region of IgGD.

TABLE 5
HINGE REGION SEQUENCES

| # | A.A. | Hinge | Sequence |
|---|---|---|---|
| 109 | √ | IgGA | FNECRCTDTPPCPVPEP |
| 110 | √ | IgGB | PKRENGRVPRPPDCPKCPAPEM |
| 111 | √ | IgGC | AKECECKCNCNNCPCPGCGL |
| 112 | √ | IgGD^{#} | PKESTCKCIPPCPVPES |

^{#}Single amino acid substitution of a serine to a proline as in bold and underlined.

TABLE 6
CANINE PD-1/PD-L1 SEQUENCES

| # | N. | A. | PD-1 | Description | # | N. | A. | PD-L1 | Description |
|---|---|---|---|---|---|---|---|---|---|
| 113 | ✓ | | ✓ | Full Length | 119 | ✓ | | ✓ | Full Length |
| 114 | | ✓ | ✓ | Full Length | 120 | | ✓ | ✓ | Full Length |
| 115 | ✓ | | ✓ | ECD | 121 | ✓ | | ✓ | ECD |
| 116 | | ✓ | ✓ | ECD | 122 | | ✓ | ✓ | ECD |
| 117 | ✓ | | ✓ | cECD-hIgG1 | 123 | ✓ | | ✓ | cECD-hIgG1 |
| 118 | | ✓ | ✓ | cECD-hIgG1 | 124 | | ✓ | ✓ | cECD-hIgG1 |
| 133 | ✓ | | ✓ | +signal seq. | 135 | ✓ | | ✓ | +signal seq. |
| 134 | | ✓ | ✓ | +signal seq. | 136 | | ✓ | ✓ | +signal seq. |
| 137 | | ✓ | ✓ | +signal seq. | | | | | |

TABLE 7
NATIVE cFc SEQUENCES

| # | N. | A. | | # | N. | A. | |
|---|---|---|---|---|---|---|---|
| 125 | ✓ | | Fc-cIgGA | 129 | ✓ | | Fc-cIgGB |
| 126 | | ✓ | Fc-cIgGA | 130 | | ✓ | Fc-cIgGB |
| 127 | ✓ | | Fc-cIgGD | 131 | ✓ | | Fc-cIgGC |
| 128 | | ✓ | Fc-cIgGD | 132 | | ✓ | Fc-cIgGC |

TABLE 8
CDR AMINO ACID SEQUENCES

| # | A.A. | CDR |
|---|---|---|
| 13 | ✓ | VL CDR1 1B5, 3B6, 4D12, 7C9 |
| 14 | ✓ | VL CDR1 2G9 |
| 15 | ✓ | VL CDR1 2H9, 5G5 |
| 16 | ✓ | VL CDR2 1B5, 7C9 |
| 17 | ✓ | VL CDR2 2G9 |
| 18 | ✓ | VL CDR2 2H9 |
| 19 | ✓ | VL CDR2 3B6 |
| 20 | ✓ | VL CDR2 4D12 |
| 21 | ✓ | VL CDR2 5G5 |
| 22 | ✓ | VL CDR3 1B5, 7C9 |
| 23 | ✓ | VL CDR3 2G9 |
| 24 | ✓ | VL CDR3 2H9 |
| 25 | ✓ | VL CDR3 4D12 |
| 26 | ✓ | VL CDR3 5G5 |
| 27 | ✓ | VH CDR1 1B5, 3B6, 4D12 |
| 28 | ✓ | VH CDR1 2G9 |
| 29 | ✓ | VH CDR1 2H9, 5G5 |
| 30 | ✓ | VH CDR1 7C9 |
| 31 | ✓ | VH CDR2 1B5, 3B6, 7C9 |
| 32 | ✓ | VH CDR2 2G9 |
| 33 | ✓ | VH CDR2 2H9 |
| 34 | ✓ | VH CDR2 4D12 |
| 35 | ✓ | VH CDR2 5G5 |
| 36 | ✓ | VH CDR3 1B5, 3B6, 4D12, 7C9 |
| 37 | ✓ | VH CDR3 2G9 |
| 38 | ✓ | VH CDR3 2H9 |
| 146 | ✓ | VH CDR3 5G5 |

TABLE 9
INDIVIDUAL SUBSTITUTED CANINIZED HEAVY CHAINS

| # | N. | A. | |
|---|---|---|---|
| 39 | ✓ | | 3B6-VH3-CH1-hinge-FC-cIgGB Fc |
| 40 | | ✓ | 3B6-VH3-CH1-hinge-FC-cIgGB Fc |
| 41 | ✓ | | 3B6-VH3-CH1-hinge-FC-cIgGC Fc |
| 42 | | ✓ | 3B6-VH3-CH1-hinge-FC-cIgGC Fc |
| 43 | ✓ | | 2H9-VH4-CH1-hinge-FC-cIgGB Fc |
| 44 | | ✓ | 2H9-VH4-CH1-hinge-FC-cIgGB Fc |
| 45 | ✓ | | 2H9-VH4-CH1-hinge-FC-cIgGC Fc |
| 46 | | ✓ | 2H9-VH4-CH1-hinge-FC-cIgGC Fc |
| 47 | ✓ | | 2G9-VH6-CH1-hinge-FC-cIgGB Fc |
| 48 | | ✓ | 2G9-VH6-CH1-hinge-FC-cIgGB Fc |
| 49 | ✓ | | 2G9-VH6-CH1-hinge-FC-cIgGC Fc |
| 50 | | ✓ | 2G9-VH6-CH1-hinge-FC-cIgGC Fc |
| 51 | ✓ | | 7C9-VH3-CH1-hinge-FC-cIgGB Fc |
| 52 | | ✓ | 7C9-VH3-CH1-hinge-FC-cIgGB Fc |
| 53 | ✓ | | 7C9-VH3-CH1-hinge-FC-cIgGC Fc |
| 54 | | ✓ | 7C9-VH3-CH1-hinge-FC-cIgGC Fc |
| 55 | ✓ | | 1B5-VH3-CH1-hinge-FC-cIgGB Fc |
| 56 | | ✓ | 1B5-VH3-CH1-hinge-FC-cIgGB Fc |
| 57 | ✓ | | 1B5-VH3-CH1-hinge-FC-cIgGC Fc |
| 58 | | ✓ | 1B5-VH3-CH1-hinge-FC-cIgGC Fc |
| 59 | ✓ | | 5G5-VH3-CH1-hinge-FC-cIgGB Fc |
| 60 | | ✓ | 5G5-VH3-CH1-hinge-FC-cIgGB Fc |
| 61 | ✓ | | 5G5-VH3-CH1-hinge-FC-cIgGC Fc |
| 62 | | ✓ | 5G5-VH3-CH1-hinge-FC-cIgGC Fc |
| 63 | ✓ | | 4D12-VH3-CH1-hinge-FC-cIgGB Fc |
| 64 | | ✓ | 4D12-VH3-CH1-hinge-FC-cIgGB Fc |
| 65 | ✓ | | 4D12-VH3-CH1-hinge-FC-cIgGC Fc |
| 66 | | ✓ | 4D12-VH3-CH1-hinge-FC-cIgGC Fc |

The potential specific substitutions are at P4, D31, N63, G64,T65, A93, and P95

TABLE 10
CORRELATION OF AMINO ACID RESIDUE POSITIONS OF
NATIVE AND SUBSTITUTED cFc WITH THAT OF THE
CORRESPONDING SUBSTITUTED CANINE HEAVY CHAINS^{#}

| 130/132 | P4 | D31 | N63 | G64 | T65 | A93 | P95 |
|---|---|---|---|---|---|---|---|
| 2/4 | 4 | 31 | 63 | 64 | 65 | 93 | 95 |
| 40 | 239 | 266 | 298 | 299 | 300 | 328 | 330 |
| 42 | 237 | 264 | 296 | 297 | 298 | 326 | 328 |
| 44 | 244 | 271 | 303 | 304 | 305 | 333 | 335 |
| 46 | 242 | 269 | 301 | 302 | 303 | 331 | 333 |
| 48 | 246 | 273 | 305 | 306 | 307 | 335 | 337 |
| 50 | 244 | 271 | 303 | 304 | 305 | 333 | 335 |
| 52 | 239 | 266 | 298 | 299 | 300 | 328 | 330 |
| 54 | 237 | 264 | 296 | 297 | 298 | 326 | 328 |
| 56 | 239 | 266 | 298 | 299 | 300 | 328 | 330 |
| 58 | 237 | 264 | 296 | 297 | 298 | 326 | 328 |
| 60 | 244 | 271 | 303 | 304 | 305 | 333 | 335 |
| 62 | 242 | 269 | 301 | 302 | 303 | 331 | 333 |
| 64 | 239 | 266 | 298 | 299 | 300 | 328 | 330 |
| 66 | 237 | 264 | 296 | 297 | 298 | 326 | 328 |

^{#}First Column lists SEQ ID NOs.; remaining columns list corresponding amino acid positions. For the two native amino acid sequences (SEQ ID NOs. 130 and 132), the one letter code for the natural amino acid residues are also provided.

TABLE 11
INDIVIDUAL UNSUBSTITUTED CANINIZED
HEAVY AND LIGHT CHAINS

| # | N. | A. | |
|---|---|---|---|
| 67 | ✓ | | 3B6-VH3-CH1-hinge-FC-cIgGA Fc |
| 68 | | ✓ | 3B6-VH3-CH1-hinge-FC-cIgGA Fc |
| 69 | ✓ | | 3B6-VH3-CH1-hinge-FC-cIgGD Fc |
| 70 | | ✓ | 3B6-VH3-CH1-hinge-FC-cIgGD Fc |
| 71 | ✓ | | 3B6-VL3-CL-Kappa |
| 72 | | ✓ | 3B6-VL3-CL-Kappa |
| 73 | ✓ | | 2H9-VH4-CH1-hinge-FC-cIgGA Fc |
| 74 | | ✓ | 2H9-VH4-CH1-hinge-FC-cIgGA Fc |
| 75 | ✓ | | 2H9-VH4-CH1-hinge-FC-cIgGD Fc |
| 76 | | ✓ | 2H9-VH4-CH1-hinge-FC-cIgGD Fc |
| 77 | ✓ | | 2H9-VL3-CL-Kappa |
| 78 | | ✓ | 2H9-VL3-CL-Kappa |
| 79 | ✓ | | 2G9-VH6-CH1-hinge-FC-cIgGA Fc |
| 80 | | ✓ | 2G9-VH6-CH1-hinge-FC-cIgGA Fc |
| 81 | ✓ | | 2G9-VH6-CH1-hinge-FC-cIgGD Fc |
| 82 | | ✓ | 2G9-VH6-CH1-hinge-FC-cIgGD Fc |
| 83 | ✓ | | 2G9-VL3-CL-Kappa |
| 84 | | ✓ | 2G9-VL3-CL-Kappa |
| 85 | ✓ | | 7C9-VH3-CH1-hinge-FC-cIgGA Fc |
| 86 | | ✓ | 7C9-VH3-CH1-hinge-FC-cIgGA Fc |
| 87 | ✓ | | 7C9-VH3-CH1-hinge-FC-cIgGD Fc |
| 88 | | ✓ | 7C9-VH3-CH1-hinge-FC-cIgGD Fc |
| 89 | ✓ | | 7C9-VL3-CL-Kappa |
| 90 | | ✓ | 7C9-VL3-CL-Kappa |
| 91 | ✓ | | 1B5-VH3-CH1-hinge-FC-cIgGA Fc |
| 92 | | ✓ | 1B5-VH3-CH1-hinge-FC-cIgGA Fc |
| 93 | ✓ | | 1B5-VH3-CH1-hinge-FC-cIgGD Fc |
| 94 | | ✓ | 1B5-VH3-CH1-hinge-FC-cIgGD Fc |
| 95 | ✓ | | 1B5-VL3-CL-Kappa |
| 96 | | ✓ | 1B5-VL3-CL-Kappa |
| 97 | ✓ | | 5G5-VH3-CH1-hinge-FC-cIgGA Fc |
| 98 | | ✓ | 5G5-VH3-CH1-hinge-FC-cIgGA Fc |
| 99 | ✓ | | 5G5-VH3-CH1-hinge-FC-cIgGD Fc |
| 100 | | ✓ | 5G5-VH3-CH1-hinge-FC-cIgGD Fc |
| 101 | ✓ | | SGS-VL3-CL-Kappa |
| 102 | | ✓ | 5G5-VL3-CL-Kappa |
| 103 | ✓ | | 4D12-VH3-CH1-hinge-FC-cIgGA Fc |
| 104 | | ✓ | 4D12-VH3-CH1-hinge-FC-cIgGA Fc |
| 105 | ✓ | | 4D12-VH3-CH1-hinge-FC-cIgGD Fc |
| 106 | | ✓ | 4D12-VH3-CH1-hinge-FC-cIgGD Fc |
| 107 | ✓ | | 4D12-VL3-CL-Kappa |
| 108 | | ✓ | 4D12-VL3-CL-Kappa |

### Example 5

### Epitope Mapping of Anti-Canine Pd-1 Antibodies

### Introduction

The interaction of antibodies with their cognate protein antigens is mediated through the binding of specific amino acids (paratopes) of the antibodies with specific amino acids (epitopes) of target antigens. An epitope is an antigenic determinant that causes a specific reaction by an immunoglobulin. It consists of a group of amino acids on the surface of the antigen.

A protein of interest may contain several epitopes that are recognized by different antibodies. The epitopes recognized by antibodies are classified as linear or conformational epitopes. Linear epitopes are formed by a stretch of continuous sequence of amino acids in a protein, while conformational epitopes are composed of amino acids that are discontinuous (e.g, far apart) in the primary amino acid sequence, but are brought together upon three-dimensional protein folding.

Epitope mapping refers to the process of identifying the amino acid sequences (i.e., epitopes) that are recognized by antibodies on their target antigens. Identification of epitopes recognized by monoclonal antibodies (mAbs) on target antigens has important applications. For example, it can aid in the development of new therapeutics, diagnostics, and vaccines. Epitope mapping can also aid in the selection of optimized therapeutic mAbs and help elucidate their mechanisms of action. Epitope information can also elucidate unique cancer epitopes and define the protective or pathogenic effects of vaccines.

Epitope mapping can be carried out using polyclonal or monoclonal antibodies and several methods are employed for epitope identification depending on the suspected nature of the epitope (i.e., linear versus conformational). Mapping linear epitopes is more straightforward and relatively easy to perform. For this purpose, commercial services for linear epitope mapping often employ peptide scanning. In this case, an overlapping set of short peptide sequences of the target protein are chemically synthesized and tested for their ability to bind antibodies of interest. The strategy is rapid, high-throughput, and relatively inexpensive to perform. On the other hand, mapping of discontinuous epitope is more technically challenging and requires more specialized techniques such as x-ray co-crystallography of a monoclonal antibody together with its target protein, Hydrogen-Deuterium (H/D) exchange, and/or Mass Spectroscopy coupled with enzymatic digestion.

### Mapping of PD-1 Epitopes Using a ProImmune® MicroArray:

In order to identify the amino acids that form the epitopes for anti-PD1 mAbs, a total of 28 peptides that are 15 amino acids long and overlapping by 10 amino acids were chemically synthesized. This library of overlapping peptides was designed to cover the full length canine PD-1 protein. The determination of peptide-antibody binding was performed by attachment of antibody samples to the ProArray Ultra® peptide microarray, followed by incubation with a fluorescent-labelled secondary antibody. All peptides are synthesized separately, and then bound to the ProArray Ultra® slide surface alongside ProImmune® murine IgG controls. This optimized process ensures that peptides are presented on the array in such a manner as to closely mimic the properties of the corresponding protein region, circumventing the inherent physiochemical variation of the free peptides themselves and making a compatible, combined peptide and protein array platform. The test analytes (peptides) are dispensed onto the ProArray Ultra® slide in discrete spots and appropriate gal-files enable exact alignment of the resulting array features back to the analyte deposited. ProArray Ultra® slides were blocked using a validated blocking buffer to reduce non-specific binding of the mAbs. They were then incubated with the mAb samples, followed by incubation with a specific fluorescent-labelled secondary antibody. After several washing steps, the ProArray Ultra® arrays were dried and scanned using a high-resolution fluorescence microarray scanning system. After scanning the fluorescent labelled ProArray Ultra® slides, the scanner recorded an image which was evaluated using image analysis software-enabling interpretation and quantification of the levels of fluorescent intensities associated with each fluorescent spot on the scanned microarray slide. The results of this experiment indicated some of the canine PD-1 peptides were recognized by some of the mAbs evaluated. The identity of the mAbs and the amino acid sequence recognized by these mAbs are listed in Table 12. This study indicates that mAb 2H9 recognizes an epitope located in the extracellular domain of canine PD-1 comprised of the amino acid sequence represented by SEQ ID NO: 138 and that mAb 1A1 recognizes an epitope comprising the amino acid sequence represented by SEQ ID NO: 138 and the overlapping amino acid sequence represented by the amino acid sequence represented by SEQ ID NO: 139.

### Mapping of PD-1 Epitopes Using Mass Spectroscopy:

In order to identify potentially discontinuous epitopes recognized by anti-canine PD-1 a method based on chemical crosslinking and mass spectrometry detection was used (CovalX® Instrument Incorporated). The application of this technology to epitope mapping of canine PD-1 resulted in identification of at least portions of epitopes recognized by the indicated mAbs which are listed in Table 13. As can be seen from Table 13, mAb 3B6 recognizes at least a portion of an epitope located in the extracellular domain of canine PD-1 within the amino acid sequence represented by SEQ ID NO: 140 and that mAb 2G9 recognizes at least a portion of an epitope within the amino acid sequence represented by SEQ ID NO: 141. On the other hand, mAb 1E4 and mAb 1B5 recognize at least a portion of an epitope within the amino acid sequence represented by SEQ ID NO: 142 and acid sequence represented by SEQ ID NO: 143, respectively.

As depicted in FIG. 9A a determination performed by chemical cross-linking, High-Mass MALDI mass spectrometry and nLC-Orbitrap mass spectrometry shows that the epitope on canine PD-1 recognized by caninized antibody 2G9 comprises R₆₂, R₆₉, R₇₂, and R₇₅ of SEQ ID NO: 114. The analogous determination for the epitope on canine PD-1 recognized by caninized antibody 3B6 comprises R₇₅ and R₉₀ of SEQ ID NO: 114. Accordingly, R₇₅ appears to be a particularly important amino acid residue in one or more epitopes of canine PD-1. Interestingly, after performing these analyses, the amino acid sequence for the CDRs of 1A1 were found to be identical to that of 2G9. The consistency between the region on PD-1 that 2G9 binds with that found for 1A1, which were obtained by these two very different methodologies, indicates that this region contains amino acid residues comprised by a PD-1 epitope that is recognized by the anti-caninine PD-1 antibodies (see, Tables 12 and 13 below).

Moreover, the region of the amino acid sequence of PD-1 that is recognized by the blocking antibodies of the present invention tested is within the extracellular domain of canine PD-1. The region recognized is comprised by the following peptide (see, Tables 12 and 13 below).

(SEQ ID NO: 144)
NQTDKLAAFQEDRIEPGRDRRFRVM*RLPNGRDFHMSIVAARLNDS

Within this peptide, is a shorter peptide that is in bold. This shorter peptide was recognized with the ProImmune® MicroArray (see, Table 12).

(SEQ ID NO: 145)
DRIEPGRDRRFRVM*RLPNGR

Notably, R₆₂, R₆₉, R₇₂, and R₇₅ of SEQ ID NO: 114 are all comprised by both the longer peptide (SEQ ID NO: 144) and the shorter peptide (SEQ ID NO: 145), whereas R₉₀ of SEQ ID NO: 114 is in the longer peptide. These five arginine residues appear to be important amino acid residues in one or more epitopes of canine PD-1. As indicated in the Tables 6-8, the starred methionine residue (*) has also been reported as being a threonine residue.

TABLE 12
PD-1 EPITOPES RECOGNIZED BY ANTI-CANINE PD-1
MAABS USING PROIMMUNE ® MICROARRAY

| | | SEQ |
|---|---|---|
| ANTIBODY | ANTIGEN PEPTIDE | ID NO: |
| 2H9 | GRDRRFRVM*RLPNGR | 138 |
| 1A1^{#} | DRIEPGRDRRFRVM*R | 139 |
| 1A1 | GRDRRFRVM*RLPNGR | 138 |

*This methionine residue has also been reported as being a threonine residue.
^{#}The CDRs of 1A1 are identical to those of 2G9.

TABLE 13
PD-1 EPITOPES RECOGNIZED BY ANTI-CANINE PD-1
MAABS USING MASS SPECTROMETRY

| | | SEQ |
|---|---|---|
| ANTIBODY | PEPTIDE ANTIGEN | ID NO: |
| 3B6 | RFRVM*RLPNGRDFHMSIVAARLNDS | 140 |
| 2G9 | LAAFQEDRIEPGRDRRFRVM*RLPNGR | 141 |
| 1E4^{#} | EDRIEPGRDRRFRVM*RLPNGRDFHMSIVAAR | 142 |
| 1B5 | NQTDKLAAFQEDRIEPGRDRRFRVM*RLPNGR | 143 |

*This methionine residue has also been reported as being a threonine residue.
^{#}The CDRs of 1E4 are most closely related to those or 2G9.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, pursuant to 37 C.F.R. § 1.57 (b) (1), to relate to each and every individual publication, database entry (e.g., Genbank sequences or GeneID entries), patent application, or patent, each of which is clearly identified in compliance with 37 C.F.R. § 1.57 (b) (2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying FIG. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

We claim:

1. An isolated nucleic acid that encodes a canine or caninized IgG heavy chain, wherein the heavy chain comprises a variable heavy chain (VH) region, the VH region comprising:
a heavy chain complementary determining region 1 (VH CDR1) comprising the amino acid sequence of SEQ ID NO: 29;
a heavy chain complementary determining region 2 (VH CDR2) comprising the amino acid sequence of SEQ ID NO: 33; and
a heavy chain complementary determining region 3 (VH CDR3) comprising the amino acid sequence of SEQ ID NO: 38.

2. The isolated nucleic acid of claim 1, wherein the canine or caninized IgG heavy chain comprises the amino acid sequence of SEQ ID NO: 44, wherein SEQ ID NO: 44 comprises: a proline residue at amino acid position 244, a glycine or alanine residue at amino acid position 271, an asparagine or alanine residue at amino acid position 303, a glycine residue at amino acid position 304, a threonine residue at amino acid position 305 and an alanine residue at amino acid position 333.

3. The isolated nucleic acid of claim 2, wherein SEQ ID NO: 44 comprises an alanine residue at amino acid position 271 or an alanine residue at amino acid position 303 of SEQ ID NO: 44, or an alanine residue at amino acid position 271 and an alanine residue at amino acid position 303 of SEQ ID NO: 44.

4. The isolated nucleic acid of claim 3, wherein the nucleic acid sequence comprises SEQ ID NO: 43 or a nucleic acid sequence having at least 85% sequence identity to SEQ ID NO: 43 and encoding the heavy chain having an amino acid sequence of SEQ ID NO: 44 having an alanine residue at amino acid position 271 or an alanine residue at amino acid position 303 of SEQ ID NO: 44, or an alanine residue at amino acid position 271 and an alanine residue at amino acid position 303 of SEQ ID NO: 44.

5. The isolated nucleic acid of claim 1, wherein the canine or caninized IgG heavy chain comprises the amino acid sequence of SEQ ID NO: 46, wherein SEQ ID NO: 46 comprises: a proline residue at amino acid position 242, a glycine or alanine residue at amino acid position 269, an asparagine or alanine residue at amino acid position 301, a glycine residue at amino acid position 302, a threonine residue at amino acid position 303 and an alanine residue at amino acid position 331.

6. The isolated nucleic acid of claim 5, wherein SEQ ID NO: 46 comprises an alanine residue at amino acid position 269 or an alanine residue at amino acid position 301 of SEQ ID NO: 46, or an alanine residue at amino acid position 269 and an alanine residue at amino acid position 301 of SEQ ID NO: 46.

7. The isolated nucleic acid of claim 6, wherein the nucleic acid sequence comprises SEQ ID NO: 45 or a nucleic acid sequence having at least 85% sequence identity to SEQ ID NO: 45 and encoding the heavy chain having an amino acid sequence of SEQ ID NO: 46 having an alanine residue at amino acid position 269 or an alanine residue at amino acid position 301 of SEQ ID NO: 46, or an alanine residue at amino acid position 269 and an alanine residue at amino acid position 301 of SEQ ID NO: 46.

8. An expression vector comprising a promoter operatively linked to the nucleic acid sequence of claim 4.

9. An isolated nucleic acid that encodes a canine or caninized light chain, wherein the light chain comprises three Variable light complementary determining regions (VL CDRs), which comprise:
a light chain complementary determining region 1 (VL CDR1) comprising the amino acid sequence of SEQ ID NO: 15;
a light chain complementary determining region 2 (VL CDR2) comprising the amino acid sequence of SEQ ID NO: 18; and
a light chain complementary determining region 3 (VL CDR3) comprising the amino acid sequence of SEQ ID NO: 24.

10. The isolated nucleic acid of claim 9, wherein the canine or caninized light chain is a caninized kappa light chain.

11. The isolated nucleic acid of claim 9, wherein the canine or caninized light chain comprises the amino acid sequence of SEQ ID NO: 78 or at least 85% sequence identity to SEQ ID NO: 78.

12. The isolated nucleic acid of claim 9, wherein the nucleic acid comprises SEQ ID NO: 77 or a sequence at least 85% sequence identity to SEQ ID NO: 77.

13. An expression vector comprising a promoter operatively linked to the nucleic acid sequence of claim 9.

14. The expression vector of claim 8, further comprising the nucleic acid sequence that encodes a canine or caninized light chain, wherein the light chain comprises three Variable light complementary determining regions (VL CDRs), which comprise: a light chain complementary determining region 1 (VL CDR1) comprising the amino acid sequence of SEQ ID NO: 15; a light chain complementary determining region 2 (VL CDR2) comprising the amino acid sequence of SEQ ID NO: 18; and a light chain complementary determining region 3 (VL CDR3) comprising the amino acid sequence of SEQ ID NO: 24.

15. A host cell comprising the expression vector of claim 8.

16. A host cell comprising the expression vector of claim 13.

17. The host cell of claim 15, further comprising a second expression vector, the second expression vector comprising the nucleic acid sequence that encodes a canine or caninized light chain, wherein the light chain comprises three Variable light complementary determining regions (VL CDRs), which comprise: a light chain complementary determining region 1 (VL CDR1) comprising the amino acid sequence of SEQ ID NO: 15; a light chain complementary determining region 2 (VL CDR2) comprising the amino acid sequence of SEQ ID NO: 18; and a light chain complementary determining region 3 (VL CDR3) comprising the amino acid sequence of SEQ ID NO: 24.

18. A host cell comprising the expression vector of claim 14.

19. The host cell of claim 15, wherein the host cell is selected from any of: an insect cell line, amphibian cell line, bacterial cell line, mammalian cell line, plant cell line and fungal cell line.

20. The host cell of claim 19, wherein the mammalian cell line is selected from any of the group consisting of: Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells and HEK-293 cells.

21. A method comprising:
culturing the host cell of claim 17 in a culture medium for a sufficient amount of time to allow expression of the antibody in the host cell, and
collecting the antibody from the culture medium.


SEQUENCE LISTING (146 sequences)

SEQ ID NO: 1 - DNA, 690 bp, synthetic construct
  misc_feature 1..690: note = modified canine sequence
  misc_difference 10..12: note = n is a, c, g, or t
  misc_difference 91..93: note = n is a, c, g, or t
  misc_difference 187..195: note = n is a, c, g, or t
  misc_difference 277..279: note = n is a, c, g, or t
  misc_difference 283..285: note = n is a, c, g, or t
  source 1..690: mol_type = other DNA
ctgggcggcnnnagcgtgtttatttttccgccgaaaccgaaagataccctgctgattgcgcgcaccccggaagtgacctgcgtggtggtgnnnctggatccggaagatccggaagtgcagattagctggtttgtggatggcaaacagatgcagaccgcgaaaacccagccgcgcgaagaacagtttnnnnnnnnntatcgcgtggtgagcgtgctgccgattggccatcaggattggctgaaaggcaaacagtttacctgcaaagtgaacaacaaannnctgnnnagcccgattgaacgcaccattagcaaagcgcgcggccaggcgcatcagccgagcgtgtatgtgctgccgccgagccgcgaagaactgagcaaaaacaccgtgagcctgacctgcctgattaaagatttttttccgccggatattgatgtggaatggcagagcaacggccagcaggaaccggaaagcaaatatcgcaccaccccgccgcagctggatgaagatggcagctattttctgtatagcaaactgagcgtggataaaagccgctggcagcgcggcgatacctttatttgcgcggtgatgcatgaagcgctgcataaccattatacccaggaaagcctgagccatagcccgggcaaacataaccattatacccaggaaagcctgagccatagcccgggcaaa

SEQ ID NO: 2 - protein, 215 aa, synthetic construct
  REGION 1..215: note = modified canine sequence
  VARIANT 4: note = misc_feature - Xaa can be any naturally occurring amino acid
  VARIANT 31: note = misc_feature - Xaa can be any naturally occurring amino acid
  VARIANT 63..65: note = misc_feature - Xaa can be any naturally occurring amino acid
  VARIANT 93: note = misc_feature - Xaa can be any naturally occurring amino acid
  VARIANT 95: note = misc_feature - Xaa can be any naturally occurring amino acid
LGGXSVFIFPPKPKDTLLIARTPEVTCVVVXLDPEDPEVQISWFVDGKQMQTAKTQPREEQFXXXYRVVSVLPIGHQDWLKGKQFTCKVNNKXLXSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 3 - DNA, 690 bp, synthetic construct
  misc_feature 1..690: note = modified canine sequence
  misc_difference 10..12: note = n is a, c, g, or t
  misc_difference 91..93: note = n is a, c, g, or t
  misc_difference 187..195: note = n is a, c, g, or t
  misc_difference 277..279: note = n is a, c, g, or t
  misc_difference 283..285: note = n is a, c, g, or t
  source 1..690: mol_type = other DNA
ctgggcggcnnnagcgtgtttatttttccgccgaaaccgaaagatattctggtgaccgcgcgcaccccgaccgtgacctgcgtggtggtgnnnctggatccggaaaacccggaagtgcagattagctggtttgtggatagcaaacaggtgcagaccgcgaacacccagccgcgcgaagaacagagcnnnnnnnnntatcgcgtggtgagcgtgctgccgattggccatcaggattggctgagcggcaaacagtttaaatgcaaagtgaacaacaaannnctgnnnagcccgattgaagaaattattagcaaaaccccgggccaggcgcatcagccgaacgtgtatgtgctgccgccgagccgcgatgaaatgagcaaaaacaccgtgaccctgacctgcctggtgaaagatttttttccgccggaaattgatgtggaatggcagagcaacggccagcaggaaccggaaagcaaatatcgcatgaccccgccgcagctggatgaagatggcagctattttctgtatagcaaactgagcgtggataaaagccgctggcagcgcggcgatacctttatttgcgcggtgatgcatgaagcgctgcataaccattatacccagattagcctgagccatagcccgggcaaacataaccattatacccagattagcctgagccatagcccgggcaaa

SEQ ID NO: 4 - protein, 215 aa, synthetic construct
  REGION 1..215: note = modified canine sequence
  VARIANT 4: note = misc_feature - Xaa can be any naturally occurring amino acid
  VARIANT 31: note = misc_feature - Xaa can be any naturally occurring amino acid
  VARIANT 63..65: note = misc_feature - Xaa can be any naturally occurring amino acid
  VARIANT 93: note = misc_feature - Xaa can be any naturally occurring amino acid
  VARIANT 95: note = misc_feature - Xaa can be any naturally occurring amino acid
LGGXSVFIFPPKPKDILVTARTPTVTCVVVXLDPENPEVQISWFVDSKQVQTANTQPREEQSXXXYRVVSVLPIGHQDWLSGKQFKCKVNNKXLXSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 5 - DNA, 699 bp, synthetic construct
  misc_feature 1..699: note = modified canine sequence
  source 1..699: mol_type = other DNA
ccgaaagaaagcacctgcaaatgcattccgccgtgcccggtgccggaaagcctgggcggcccgagcgtgtttatttttccgccgaaaccgaaagatattctgcgcattacccgcaccccggaaattacctgcgtggtgctggatctgggccgcgaagatccggaagtgcagattagctggtttgtggatggcaaagaagtgcataccgcgaaaacccagccgcgcgaacagcagtttaacagcacctatcgcgtggtgagcgtgctgccgattgaacatcaggattggctgaccggcaaagaatttaaatgccgcgtgaaccatattggcctgccgagcccgattgaacgcaccattagcaaagcgcgcggccaggcgcatcagccgagcgtgtatgtgctgccgccgagcccgaaagaactgagcagcagcgataccgtgaccctgacctgcctgattaaagatttttttccgccggaaattgatgtggaatggcagagcaacggccagccggaaccggaaagcaaatatcataccaccgcgccgcagctggatgaagatggcagctattttctgtatagcaaactgagcgtggataaaagccgctggcagcagggcgatacctttacctgcgcggtgatgcatgaagcgctgcagaaccattataccgatctgagcctgagccatagcccgggcaaa

SEQ ID NO: 6 - protein, 233 aa, synthetic construct
  REGION 1..233: note = modified canine sequence
PKESTCKCIPPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 7 - DNA, 699 bp, synthetic construct
  misc_feature 1..699: note = modified canine sequence
  source 1..699: mol_type = other DNA
tttaacgaatgccgctgcaccgataccccgccgtgcccggtgccggaaccgctgggcggcccgagcgtgtttatttttccgccgaaaccgaaagatattctgcgcattacccgcaccccggaaattacctgcgtggtgctggatctgggccgcgaagatccggaagtgcagattagctggtttgtggatggcaaagaagtgcataccgcgaaaacccagccgcgcgaacagcagtttaacagcacctatcgcgtggtgagcgtgctgccgattgaacatcaggattggctgaccggcaaagaatttaaatgccgcgtgaaccatattggcctgccgagcccgattgaacgcaccattagcaaagcgcgcggccaggcgcatcagccgagcgtgtatgtgctgccgccgagcccgaaagaactgagcagcagcgataccgtgaccctgacctgcctgattaaagatttttttccgccggaaattgatgtggaatggcagagcaacggccagccggaaccggaaagcaaatatcataccaccgcgccgcagctggatgaagatggcagctattttctgtatagcaaactgagcgtggataaaagccgctggcagcagggcgatacctttacctgcgcggtgatgcatgaagcgctgcagaaccattataccgatctgagcctgagccatagcccgggcaaa

SEQ ID NO: 8 - protein, 233 aa, synthetic construct
  REGION 1..233: note = modified canine sequence
FNECRCTDTPPCPVPEPLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 9 - DNA, 714 bp, synthetic construct
  misc_feature 1..714: note = modified canine sequence
  source 1..714: mol_type = other DNA
ccgaaacgcgaaaacggccgcgtgccgcgcccgccggattgcccgaaatgcccggcgccggaaatgctgggcggcccgagcgtgtttatttttccgccgaaaccgaaagatattctgcgcattacccgcaccccggaaattacctgcgtggtgctggatctgggccgcgaagatccggaagtgcagattagctggtttgtggatggcaaagaagtgcataccgcgaaaacccagccgcgcgaacagcagtttaacagcacctatcgcgtggtgagcgtgctgccgattgaacatcaggattggctgaccggcaaagaatttaaatgccgcgtgaaccatattggcctgccgagcccgattgaacgcaccattagcaaagcgcgcggccaggcgcatcagccgagcgtgtatgtgctgccgccgagcccgaaagaactgagcagcagcgataccgtgaccctgacctgcctgattaaagatttttttccgccggaaattgatgtggaatggcagagcaacggccagccggaaccggaaagcaaatatcataccaccgcgccgcagctggatgaagatggcagctattttctgtatagcaaactgagcgtggataaaagccgctggcagcagggcgatacctttacctgcgcggtgatgcatgaagcgctgcagaaccattataccgatctgagcctgagccatagcccgggcaaa

SEQ ID NO: 10 - protein, 238 aa, synthetic construct
  REGION 1..238: note = modified canine sequence
PKRENGRVPRPPDCPKCPAPEMLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 11 - DNA, 708 bp, synthetic construct
  misc_feature 1..708: note = modified canine sequence
  source 1..708: mol_type = other DNA
gccaaggagtgcgagtgcaagtgcaactgcaacaactgcccctgccccggctgcggcctgctgggcggccccagcgtgttcatcttcccccccaagcccaaggacatcctgagaatcaccagaacccccgagatcacctgcgtggtgctggacctgggcagagaggaccccgaggtgcagatcagctggttcgtggacggcaaggaggtgcacaccgccaagacccagcccagagagcagcagttcaacagcacctacagagtggtgagcgtgctgcccatcgagcaccaggactggctgaccggcaaggagttcaagtgcagagtgaaccacatcggcctgcccagccccatcgagagaaccatcagcaaggccagaggccaggcccaccagcccagcgtgtacgtgctgccccccagccccaaggagctgagcagcagcgacaccgtgaccctgacctgcctgatcaaggacttcttcccccccgagatcgacgtggagtggcagagcaacggccagcccgagcccgagagcaagtaccacaccaccgccccccagctggacgaggacggcagctacttcctgtacagcaagctgagcgtggacaagagcagatggcagcagggcgacaccttcacctgcgccgtgatgcacgaggccctgcagaaccactacaccgacctgagcctgagccacagccccggcaag

SEQ ID NO: 12 - protein, 236 aa, synthetic construct
  REGION 1..236: note = modified canine sequence
AKECECKCNCNNCPCPGCGLLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 13 - protein, 17 aa, Mus musculus
KSSQSLLNSVNQKNYLA

SEQ ID NO: 14 - protein, 16 aa, Mus musculus
RSSQNIVHSNGNTYLE

SEQ ID NO: 15 - protein, 11 aa, Mus musculus
HASQNINVWLS

SEQ ID NO: 16 - protein, 7 aa, Mus musculus
FASTRVS

SEQ ID NO: 17 - protein, 7 aa, Mus musculus
KVSNRFS

SEQ ID NO: 18 - protein, 7 aa, Mus musculus
KASHLHT

SEQ ID NO: 19 - protein, 7 aa, Mus musculus
FASARVS

SEQ ID NO: 20 - protein, 7 aa, Mus musculus
FASTRIS

SEQ ID NO: 21 - protein, 7 aa, Mus musculus
KASNLHT

SEQ ID NO: 22 - protein, 9 aa, Mus musculus
QQYFSTPLT

SEQ ID NO: 23 - protein, 9 aa, Mus musculus
FQGSHVPYT

SEQ ID NO: 24 - protein, 9 aa, Mus musculus
QQGQSWPLT

SEQ ID NO: 25 - protein, 9 aa, Mus musculus
QQYFSTPLT

SEQ ID NO: 26 - protein, 9 aa, Mus musculus
QQGQSYPLT

SEQ ID NO: 27 - protein, 10 aa, Mus musculus
GYTFTTYGMS

SEQ ID NO: 28 - protein, 10 aa, Mus musculus
GYTFTRYNMH

SEQ ID NO: 29 - protein, 10 aa, Mus musculus
GFNIKNTYMH

SEQ ID NO: 30 - protein, 10 aa, Mus musculus
GFSLTSYGVH

SEQ ID NO: 31 - protein, 17 aa, Mus musculus
WINIYSGIPTYADDFKG

SEQ ID NO: 32 - protein, 17 aa, Mus musculus
TIYPGYGDTSYNQKFKG

SEQ ID NO: 33 - protein, 17 aa, Mus musculus
RIAPANVDTKYAPKFQG

SEQ ID NO: 34 - protein, 17 aa, Mus musculus
WINIYSGMPTYADDFKG

SEQ ID NO: 35 - protein, 17 aa, Mus musculus
RIDPANVNTKYAPKFQG

SEQ ID NO: 36 - protein, 6 aa, Mus musculus
FDGPDY

SEQ ID NO: 37 - protein, 13 aa, Mus musculus
EFADDYPIPPFDY

SEQ ID NO: 38 - protein, 11 aa, Mus musculus
IYYDYDGDIDV

SEQ ID NO: 39 - DNA, 1350 bp, synthetic construct
  misc_feature 1..1350: note = caninized mouse antibody
  misc_difference 715..717: note = n is a, c, g, or t
  misc_difference 796..798: note = n is a, c, g, or t
  misc_difference 892..900: note = n is a, c, g, or t
  misc_difference 982..984: note = n is a, c, g, or t
  misc_difference 988..990: note = n is a, c, g, or t
  source 1..1350: mol_type = other DNA
gaggtgcagctggtgcagagcggcggcgacctggtgaagcccggcggcagcgtgagactgagctgcgtggccagcggctacaccttcaccacctacggcatgagctgggtgagacaggcccccggcaagggcctgcagtggatgggctggatcaacatctacagcggcatccccacctacgccgacgacttcaagggcagattcaccttcagcctggacaccgccaagaacaccgcctacctgcagctgaacagcctgagagccgaggacaccgccgtgtactactgcaccagattcgacggccccgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcatggtgaccgtgcccagcagcagatggcccagcgagaccttcacctgcaacgtggcccaccccgccagcaagaccaaggtggacaagcccgtgcccaagagagagaacggcagagtgcccagaccccccgactgccccaagtgccccgcccccgagatgctgggcggcnnnagcgtgttcatcttcccccccaagcccaaggacaccctgctgatcgccagaacccccgaggtgacctgcgtggtggtgnnnctggaccccgaggaccccgaggtgcagatcagctggttcgtggacggcaagcagatgcagaccgccaagacccagcccagagaggagcagttcnnnnnnnnntacagagtggtgagcgtgctgcccatcggccaccaggactggctgaagggcaagcagttcacctgcaaggtgaacaacaagnnnctgnnnagccccatcgagagaaccatcagcaaggccagaggccaggcccaccagcccagcgtgtacgtgctgccccccagcagagaggagctgagcaagaacaccgtgagcctgacctgcctgatcaaggacttcttcccccccgacatcgacgtggagtggcagagcaacggccagcaggagcccgagagcaagtacagaaccacccccccccagctggacgaggacggcagctacttcctgtacagcaagctgagcgtggacaagagcagatggcagagaggcgacaccttcatctgcgccgtgatgcacgaggccctgcacaaccactacacccaggagagcctgagccacagccccggcaag

SEQ ID NO: 40 - protein, 450 aa, synthetic construct
  REGION 1..450: note = caninized mouse antibody
  VARIANT 239: note = misc_feature - Xaa can be any naturally occurring amino acid
  VARIANT 266: note = misc_feature - Xaa can be any naturally occurring amino acid
  VARIANT 298..300: note = misc_feature - Xaa can be any naturally occurring amino acid
  VARIANT 328: note = misc_feature - Xaa can be any naturally occurring amino acid
  VARIANT 330: note = misc_feature - Xaa can be any naturally occurring amino acid
EVQLVQSGGDLVKPGGSVRLSCVASGYTFTTYGMSWVRQAPGKGLQWMGWINIYSGIPTYADDFKGRFTFSLDTAKNTAYLQLNSLRAEDTAVYYCTRFDGPDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGXSVFIFPPKPKDTLLIARTPEVTCVVVXLDPEDPEVQISWFVDGKQMQTAKTQPREEQFXXXYRVVSVLPIGHQDWLKGKQFTCKVNNKXLXSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 41 - DNA, 1344 bp, synthetic construct
  misc_feature 1..1344: note = caninized mouse antibody
  misc_difference 709..711: note = n is a, c, g, or t
  misc_difference 790..792: note = n is a, c, g, or t
  misc_difference 886..894: note = n is a, c, g, or t
  misc_difference 976..978: note = n is a, c, g, or t
  misc_difference 982..984: note = n is a, c, g, or t
  source 1..1344: mol_type = other DNA
gaggtgcagctggtgcagagcggcggcgacctggtgaagcccggcggcagcgtgagactgagctgcgtggccagcggctacaccttcaccacctacggcatgagctgggtgagacaggcccccggcaagggcctgcagtggatgggctggatcaacatctacagcggcatccccacctacgccgacgacttcaagggcagattcaccttcagcctggacaccgccaagaacaccgcctacctgcagctgaacagcctgagagccgaggacaccgccgtgtactactgcaccagattcgacggccccgactactggggccagggcaccctggtgaccgtgagcagcgccagcaccaccgcccccagcgtgttccccctggcccccagctgcggcagccagagcggcagcaccgtggccctggcctgcctggtgagcggctacatccccgagcccgtgaccgtgagctggaacagcgtgagcctgaccagcggcgtgcacaccttccccagcgtgctgcagagcagcggcctgtacagcctgagcagcatggtgaccgtgcccagcagcagatggcccagcgagaccttcacctgcaacgtggcccaccccgccaccaacaccaaggtggacaagcccgtggccaaggagtgcgagtgcaagtgcaactgcaacaactgcccctgccccggctgcggcctgctgggcggcnnnagcgtgttcatcttcccccccaagcccaaggacatcctggtgaccgccagaacccccaccgtgacctgcgtggtggtgnnnctggaccccgagaaccccgaggtgcagatcagctggttcgtggacagcaagcaggtgcagaccgccaacacccagcccagagaggagcagagcnnnnnnnnntacagagtggtgagcgtgctgcccatcggccaccaggactggctgagcggcaagcagttcaagtgcaaggtgaacaacaagnnnctgnnnagccccatcgaggagatcatcagcaagacccccggccaggcccaccagcccaacgtgtacgtgctgccccccagcagagacgagatgagcaagaacaccgtgaccctgacctgcctggtgaaggacttcttcccccccgagatcgacgtggagtggcagagcaacggccagcaggagcccgagagcaagtacagaatgacccccccccagctggacgaggacggcagctacttcctgtacagcaagctgagcgtggacaagagcagatggcagagaggcgacaccttcatctgcgccgtgatgcacgaggccctgcacaaccactacacccagatcagcctgagccacagccccggcaag

SEQ ID NO: 42 - protein, 448 aa, synthetic construct
  REGION 1..448: note = caninized mouse antibody
  VARIANT 237: note = misc_feature - Xaa can be any naturally occurring amino acid
  VARIANT 264: note = misc_feature - Xaa can be any naturally occurring amino acid
  VARIANT 296..298: note = misc_feature - Xaa can be any naturally occurring amino acid
  VARIANT 326: note = misc_feature - Xaa can be any naturally occurring amino acid
  VARIANT 328: note = misc_feature - Xaa can be any naturally occurring amino acid
EVQLVQSGGDLVKPGGSVRLSCVASGYTFTTYGMSWVRQAPGKGLQWMGWINIYSGIPTYADDFKGRFTFSLDTAKNTAYLQLNSLRAEDTAVYYCTRFDGPDYWGQGTLVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGXSVFIFPPKPKDILVTARTPTVTCVVVXLDPENPEVQISWFVDSKQVQTANTQPREEQSXXXYRVVSVLPIGHQDWLSGKQFKCKVNNKXLXSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 43 - DNA, 1365 bp, synthetic construct
  misc_feature 1..1365: note = caninized mouse antibody
  misc_difference 730..732: note = n is a, c, g, or t
  misc_difference 811..813: note = n is a, c, g, or t
  misc_difference 907..915: note = n is a, c, g, or t
  misc_difference 997..999: note = n is a, c, g, or t
  misc_difference 1003..1005: note = n is a, c, g, or t
  source 1..1365: mol_type = other DNA
gaggtgcagctggtgcagagcggcggcgacctggtgaagcccggcggcagcgtgagactgagctgcgtggccagcggcttcaacatcaagaacacctacatgcactgggtgagacaggcccccggcaagggcctgcagtggatcggcagaatcgcccccgccaacgtggacaccaagtacgcccccaagttccagggcaaggccaccatcagcgccgacaccgccaagaacaccgcctacatgcagctgaacagcctgagagccgaggacaccgccgtgtactactgcgtgctgatctactacgactacgacggcgacatcgacgtgtggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcatggtgaccgtgcccagcagcagatggcccagcgagaccttcacctgcaacgtggcccaccccgccagcaagaccaaggtggacaagcccgtgcccaagagagagaacggcagagtgcccagaccccccgactgccccaagtgccccgcccccgagatgctgggcggcnnnagcgtgttcatcttcccccccaagcccaaggacaccctgctgatcgccagaacccccgaggtgacctgcgtggtggtgnnnctggaccccgaggaccccgaggtgcagatcagctggttcgtggacggcaagcagatgcagaccgccaagacccagcccagagaggagcagttcnnnnnnnnntacagagtggtgagcgtgctgcccatcggccaccaggactggctgaagggcaagcagttcacctgcaaggtgaacaacaagnnnctgnnnagccccatcgagagaaccatcagcaaggccagaggccaggcccaccagcccagcgtgtacgtgctgccccccagcagagaggagctgagcaagaacaccgtgagcctgacctgcctgatcaaggacttcttcccccccgacatcgacgtggagtggcagagcaacggccagcaggagcccgagagcaagtacagaaccacccccccccagctggacgaggacggcagctacttcctgtacagcaagctgagcgtggacaagagcagatggcagagaggcgacaccttcatctgcgccgtgatgcacgaggccctgcacaaccactacacccaggagagcctgagccacagccccggcaag

SEQ ID NO: 44 - protein, 455 aa, synthetic construct
  REGION 1..455: note = caninized mouse antibody
  VARIANT 244: note = misc_feature - Xaa can be any naturally occurring amino acid
  VARIANT 271: note = misc_feature - Xaa can be any naturally occurring amino acid
  VARIANT 303..305: note = misc_feature - Xaa can be any naturally occurring amino acid
  VARIANT 333: note = misc_feature - Xaa can be any naturally occurring amino acid
  VARIANT 335: note = misc_feature - Xaa can be any naturally occurring amino acid
EVQLVQSGGDLVKPGGSVRLSCVASGFNIKNTYMHWVRQAPGKGLQWIGRIAPANVDTKYAPKFQGKATISADTAKNTAYMQLNSLRAEDTAVYYCVLIYYDYDGDIDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGXSVFIFPPKPKDTLLIARTPEVTCVVVXLDPEDPEVQISWFVDGKQMQTAKTQPREEQFXXXYRVVSVLPIGHQDWLKGKQFTCKVNNKXLXSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 45 - DNA, 1359 bp, synthetic construct
  misc_feature 1..1359: note = caniinized mouse antibody
  misc_difference 724..726: note = n is a, c, g, or t
  misc_difference 805..807: note = n is a, c, g, or t
  misc_difference 901..909: note = n is a, c, g, or t
  misc_difference 991..993: note = n is a, c, g, or t
  misc_difference 997..999: note = n is a, c, g, or t
  source 1..1359: mol_type = other DNA
gaggtgcagctggtgcagagcggcggcgacctggtgaagcccggcggcagcgtgagactgagctgcgtggccagcggcttcaacatcaagaacacctacatgcactgggtgagacaggcccccggcaagggcctgcagtggatcggcagaatcgcccccgccaacgtggacaccaagtacgcccccaagttccagggcaaggccaccatcagcgccgacaccgccaagaacaccgcctacatgcagctgaacagcctgagagccgaggacaccgccgtgtactactgcgtgctgatctactacgactacgacggcgacatcgacgtgtggggccagggcaccctggtgaccgtgagcagcgccagcaccaccgcccccagcgtgttccccctggcccccagctgcggcagccagagcggcagcaccgtggccctggcctgcctggtgagcggctacatccccgagcccgtgaccgtgagctggaacagcgtgagcctgaccagcggcgtgcacaccttccccagcgtgctgcagagcagcggcctgtacagcctgagcagcatggtgaccgtgcccagcagcagatggcccagcgagaccttcacctgcaacgtggcccaccccgccaccaacaccaaggtggacaagcccgtggccaaggagtgcgagtgcaagtgcaactgcaacaactgcccctgccccggctgcggcctgctgggcggcnnnagcgtgttcatcttcccccccaagcccaaggacatcctggtgaccgccagaacccccaccgtgacctgcgtggtggtgnnnctggaccccgagaaccccgaggtgcagatcagctggttcgtggacagcaagcaggtgcagaccgccaacacccagcccagagaggagcagagcnnnnnnnnntacagagtggtgagcgtgctgcccatcggccaccaggactggctgagcggcaagcagttcaagtgcaaggtgaacaacaagnnnctgnnnagccccatcgaggagatcatcagcaagacccccggccaggcccaccagcccaacgtgtacgtgctgccccccagcagagacgagatgagcaagaacaccgtgaccctgacctgcctggtgaaggacttcttcccccccgagatcgacgtggagtggcagagcaacggccagcaggagcccgagagcaagtacagaatgacccccccccagctggacgaggacggcagctacttcctgtacagcaagctgagcgtggacaagagcagatggcagagaggcgacaccttcatctgcgccgtgatgcacgaggccctgcacaaccactacacccagatcagcctgagccacagccccggcaag

SEQ ID NO: 46 - protein, 453 aa, synthetic construct
  REGION 1..453: note = caninized mouse antibody
  VARIANT 242: note = misc_feature - Xaa can be any naturally occurring amino acid
  VARIANT 269: note = misc_feature - Xaa can be any naturally occurring amino acid
  VARIANT 301..303: note = misc_feature - Xaa can be any naturally occurring amino acid
  VARIANT 331: note = misc_feature - Xaa can be any naturally occurring amino acid
  VARIANT 333: note = misc_feature - Xaa can be any naturally occurring amino acid
EVQLVQSGGDLVKPGGSVRLSCVASGFNIKNTYMHWVRQAPGKGLQWIGRIAPANVDTKYAPKFQGKATISADTAKNTAYMQLNSLRAEDTAVYYCVLIYYDYDGDIDVWGQGTLVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGXSVFIFPPKPKDILVTARTPTVTCVVVXLDPENPEVQISWFVDSKQVQTANTQPREEQSXXXYRVVSVLPIGHQDWLSGKQFKCKVNNKXLXSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 47 - DNA, 1371 bp, synthetic construct
  misc_feature 1..1371: note = caninized mouse antibody
  misc_difference 736..738: note = n is a, c, g, or t
  misc_difference 817..819: note = n is a, c, g, or t
  misc_difference 913..921: note = n is a, c, g, or t
  misc_difference 1003..1005: note = n is a, c, g, or t
  misc_difference 1009..1011: note = n is a, c, g, or t
  source 1..1371: mol_type = other DNA
gaggtgcagctggtgcagagcggcggcgacctggtgaagcccggcggcagcgtgagactgagctgcgtggccagcggctacaccttcaccagatacaacatgcactgggtgagacaggcccccggcaagggcctgcagtggatcggcaccatctaccccggctacggcgacaccagctacaaccagaagttcaagggcaaggccaccctgagcgtggacatcgccaagaacaccgcctacatgcagctgaacagcctgagagccgaggacaccgccgtgtacttctgcagcagagagttcgccgacgactaccccatcccccccttcgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcatggtgaccgtgcccagcagcagatggcccagcgagaccttcacctgcaacgtggcccaccccgccagcaagaccaaggtggacaagcccgtgcccaagagagagaacggcagagtgcccagaccccccgactgccccaagtgccccgcccccgagatgctgggcggcnnnagcgtgttcatcttcccccccaagcccaaggacaccctgctgatcgccagaacccccgaggtgacctgcgtggtggtgnnnctggaccccgaggaccccgaggtgcagatcagctggttcgtggacggcaagcagatgcagaccgccaagacccagcccagagaggagcagttcnnnnnnnnntacagagtggtgagcgtgctgcccatcggccaccaggactggctgaagggcaagcagttcacctgcaaggtgaacaacaagnnnctgnnnagccccatcgagagaaccatcagcaaggccagaggccaggcccaccagcccagcgtgtacgtgctgccccccagcagagaggagctgagcaagaacaccgtgagcctgacctgcctgatcaaggacttcttcccccccgacatcgacgtggagtggcagagcaacggccagcaggagcccgagagcaagtacagaaccacccccccccagctggacgaggacggcagctacttcctgtacagcaagctgagcgtggacaagagcagatggcagagaggcgacaccttcatctgcgccgtgatgcacgaggccctgcacaaccactacacccaggagagcctgagccacagccccggcaag

SEQ ID NO: 48 - protein, 457 aa, synthetic construct
  REGION 1..457: note = caninized mouse antibody
  VARIANT 246: note = misc_feature - Xaa can be any naturally occurring amino acid
  VARIANT 273: note = misc_feature - Xaa can be any naturally occurring amino acid
  VARIANT 305..307: note = misc_feature - Xaa can be any naturally occurring amino acid
  VARIANT 335: note = misc_feature - Xaa can be any naturally occurring amino acid
  VARIANT 337: note = misc_feature - Xaa can be any naturally occurring amino acid
EVQLVQSGGDLVKPGGSVRLSCVASGYTFTRYNMHWVRQAPGKGLQWIGTIYPGYGDTSYNQKFKGKATLSVDIAKNTAYMQLNSLRAEDTAVYFCSREFADDYPIPPFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGXSVFIFPPKPKDTLLIARTPEVTCVVVXLDPEDPEVQISWFVDGKQMQTAKTQPREEQFXXXYRVVSVLPIGHQDWLKGKQFTCKVNNKXLXSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 49 - DNA, 1365 bp, synthetic construct
  misc_feature 1..1365: note = caninized mouse antibody
  misc_difference 730..732: note = n is a, c, g, or t
  misc_difference 811..813: note = n is a, c, g, or t
  misc_difference 907..915: note = n is a, c, g, or t
  misc_difference 997..999: note = n is a, c, g, or t
  misc_difference 1003..1005: note = n is a, c, g, or t
  source 1..1365: mol_type = other DNA
gaggtgcagctggtgcagagcggcggcgacctggtgaagcccggcggcagcgtgagactgagctgcgtggccagcggctacaccttcaccagatacaacatgcactgggtgagacaggcccccggcaagggcctgcagtggatcggcaccatctaccccggctacggcgacaccagctacaaccagaagttcaagggcaaggccaccctgagcgtggacatcgccaagaacaccgcctacatgcagctgaacagcctgagagccgaggacaccgccgtgtacttctgcagcagagagttcgccgacgactaccccatcccccccttcgactactggggccagggcaccctggtgaccgtgagcagcgccagcaccaccgcccccagcgtgttccccctggcccccagctgcggcagccagagcggcagcaccgtggccctggcctgcctggtgagcggctacatccccgagcccgtgaccgtgagctggaacagcgtgagcctgaccagcggcgtgcacaccttccccagcgtgctgcagagcagcggcctgtacagcctgagcagcatggtgaccgtgcccagcagcagatggcccagcgagaccttcacctgcaacgtggcccaccccgccaccaacaccaaggtggacaagcccgtggccaaggagtgcgagtgcaagtgcaactgcaacaactgcccctgccccggctgcggcctgctgggcggcnnnagcgtgttcatcttcccccccaagcccaaggacatcctggtgaccgccagaacccccaccgtgacctgcgtggtggtgnnnctggaccccgagaaccccgaggtgcagatcagctggttcgtggacagcaagcaggtgcagaccgccaacacccagcccagagaggagcagagcnnnnnnnnntacagagtggtgagcgtgctgcccatcggccaccaggactggctgagcggcaagcagttcaagtgcaaggtgaacaacaagnnnctgnnnagccccatcgaggagatcatcagcaagacccccggccaggcccaccagcccaacgtgtacgtgctgccccccagcagagacgagatgagcaagaacaccgtgaccctgacctgcctggtgaaggacttcttcccccccgagatcgacgtggagtggcagagcaacggccagcaggagcccgagagcaagtacagaatgacccccccccagctggacgaggacggcagctacttcctgtacagcaagctgagcgtggacaagagcagatggcagagaggcgacaccttcatctgcgccgtgatgcacgaggccctgcacaaccactacacccagatcagcctgagccacagccccggcaag

SEQ ID NO: 50 - protein, 455 aa, synthetic construct
  REGION 1..455: note = caninized mouse antibody
  VARIANT 244: note = misc_feature - Xaa can be any naturally occurring amino acid
  VARIANT 271: note = misc_feature - Xaa can be any naturally occurring amino acid
  VARIANT 303..305: note = misc_feature - Xaa can be any naturally occurring amino acid
  VARIANT 333: note = misc_feature - Xaa can be any naturally occurring amino acid
  VARIANT 335: note = misc_feature - Xaa can be any naturally occurring amino acid
EVQLVQSGGDLVKPGGSVRLSCVASGYTFTRYNMHWVRQAPGKGLQWIGTIYPGYGDTSYNQKFKGKATLSVDIAKNTAYMQLNSLRAEDTAVYFCSREFADDYPIPPFDYWGQGTLVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGXSVFIFPPKPKDILVTARTPTVTCVVVXLDPENPEVQISWFVDSKQVQTANTQPREEQSXXXYRVVSVLPIGHQDWLSGKQFKCKVNNKXLXSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 51 - DNA, 1350 bp, synthetic construct
  misc_feature 1..1350: note = caninized mouse antibody
  misc_difference 715..717: note = n is a, c, g, or t
  misc_difference 796..798: note = n is a, c, g, or t
  misc_difference 892..900: note = n is a, c, g, or t
  misc_difference 982..984: note = n is a, c, g, or t
  misc_difference 988..990: note = n is a, c, g, or t
  source 1..1350: mol_type = other DNA
gaggtgcagctggtgcagagcggccccggcctggtgaagcccagccagagcctgagcctgacctgcgtggtgagcggcttcagcctgaccagctacggcgtgcactgggtgagacaggcccccggcaagggcctgcagtggatgggctggatcaacatctacagcggcatccccacctacgccgacgacttcaagggcagattcaccttcagcctggacaccgccaagaacaccgcctacctgcagctgagcagcctgagagccgaggacaccgccgtgtactactgcgccagattcgacggccccgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcatggtgaccgtgcccagcagcagatggcccagcgagaccttcacctgcaacgtggcccaccccgccagcaagaccaaggtggacaagcccgtgcccaagagagagaacggcagagtgcccagaccccccgactgccccaagtgccccgcccccgagatgctgggcggcnnnagcgtgttcatcttcccccccaagcccaaggacaccctgctgatcgccagaacccccgaggtgacctgcgtggtggtgnnnctggaccccgaggaccccgaggtgcagatcagctggttcgtggacggcaagcagatgcagaccgccaagacccagcccagagaggagcagttcnnnnnnnnntacagagtggtgagcgtgctgcccatcggccaccaggactggctgaagggcaagcagttcacctgcaaggtgaacaacaagnnnctgnnnagccccatcgagagaaccatcagcaaggccagaggccaggcccaccagcccagcgtgtacgtgctgccccccagcagagaggagctgagcaagaacaccgtgagcctgacctgcctgatcaaggacttcttcccccccgacatcgacgtggagtggcagagcaacggccagcaggagcccgagagcaagtacagaaccacccccccccagctggacgaggacggcagctacttcctgtacagcaagctgagcgtggacaagagcagatggcagagaggcgacaccttcatctgcgccgtgatgcacgaggccctgcacaaccactacacccaggagagcctgagccacagccccggcaag

SEQ ID NO: 52 - protein, 450 aa, synthetic construct
  REGION 1..450: note = caninized mouse antibody
  VARIANT 239: note = misc_feature - Xaa can be any naturally occurring amino acid
  VARIANT 266: note = misc_feature - Xaa can be any naturally occurring amino acid
  VARIANT 298..300: note = misc_feature - Xaa can be any naturally occurring amino acid
  VARIANT 328: note = misc_feature - Xaa can be any naturally occurring amino acid
  VARIANT 330: note = misc_feature - Xaa can be any naturally occurring amino acid
EVQLVQSGPGLVKPSQSLSLTCVVSGFSLTSYGVHWVRQAPGKGLQWMGWINIYSGIPTYADDFKGRFTFSLDTAKNTAYLQLSSLRAEDTAVYYCARFDGPDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGXSVFIFPPKPKDTLLIARTPEVTCVVVXLDPEDPEVQISWFVDGKQMQTAKTQPREEQFXXXYRVVSVLPIGHQDWLKGKQFTCKVNNKXLXSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 53 - DNA, 1344 bp, synthetic construct
  misc_feature 1..1344: note = caninized mouse antibody
  misc_difference 709..711: note = n is a, c, g, or t
  misc_difference 790..792: note = n is a, c, g, or t
  misc_difference 886..894: note = n is a, c, g, or t
  misc_difference 976..978: note = n is a, c, g, or t
  misc_difference 982..984: note = n is a, c, g, or t
  source 1..1344: mol_type = other DNA
gaggtgcagctggtgcagagcggccccggcctggtgaagcccagccagagcctgagcctgacctgcgtggtgagcggcttcagcctgaccagctacggcgtgcactgggtgagacaggcccccggcaagggcctgcagtggatgggctggatcaacatctacagcggcatccccacctacgccgacgacttcaagggcagattcaccttcagcctggacaccgccaagaacaccgcctacctgcagctgagcagcctgagagccgaggacaccgccgtgtactactgcgccagattcgacggccccgactactggggccagggcaccctggtgaccgtgagcagcgccagcaccaccgcccccagcgtgttccccctggcccccagctgcggcagccagagcggcagcaccgtggccctggcctgcctggtgagcggctacatccccgagcccgtgaccgtgagctggaacagcgtgagcctgaccagcggcgtgcacaccttccccagcgtgctgcagagcagcggcctgtacagcctgagcagcatggtgaccgtgcccagcagcagatggcccagcgagaccttcacctgcaacgtggcccaccccgccaccaacaccaaggtggacaagcccgtggccaaggagtgcgagtgcaagtgcaactgcaacaactgcccctgccccggctgcggcctgctgggcggcnnnagcgtgttcatcttcccccccaagcccaaggacatcctggtgaccgccagaacccccaccgtgacctgcgtggtggtgnnnctggaccccgagaaccccgaggtgcagatcagctggttcgtggacagcaagcaggtgcagaccgccaacacccagcccagagaggagcagagcnnnnnnnnntacagagtggtgagcgtgctgcccatcggccaccaggactggctgagcggcaagcagttcaagtgcaaggtgaacaacaagnnnctgnnnagccccatcgaggagatcatcagcaagacccccggccaggcccaccagcccaacgtgtacgtgctgccccccagcagagacgagatgagcaagaacaccgtgaccctgacctgcctggtgaaggacttcttcccccccgagatcgacgtggagtggcagagcaacggccagcaggagcccgagagcaagtacagaatgacccccccccagctggacgaggacggcagctacttcctgtacagcaagctgagcgtggacaagagcagatggcagagaggcgacaccttcatctgcgccgtgatgcacgaggccctgcacaaccactacacccagatcagcctgagccacagccccggcaag

SEQ ID NO: 54 - protein, 448 aa, synthetic construct
  REGION 1..448: note = caninized mouse antibody
  VARIANT 237: note = misc_feature - Xaa can be any naturally occurring amino acid
  VARIANT 264: note = misc_feature - Xaa can be any naturally occurring amino acid
  VARIANT 296..298: note = misc_feature - Xaa can be any naturally occurring amino acid
  VARIANT 326: note = misc_feature - Xaa can be any naturally occurring amino acid
  VARIANT 328: note = misc_feature - Xaa can be any naturally occurring amino acid
EVQLVQSGPGLVKPSQSLSLTCVVSGFSLTSYGVHWVRQAPGKGLQWMGWINIYSGIPTYADDFKGRFTFSLDTAKNTAYLQLSSLRAEDTAVYYCARFDGPDYWGQGTLVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGXSVFIFPPKPKDILVTARTPTVTCVVVXLDPENPEVQISWFVDSKQVQTANTQPREEQSXXXYRVVSVLPIGHQDWLSGKQFKCKVNNKXLXSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 55 - DNA, 1350 bp, synthetic construct
  misc_feature 1..1350: note = caninized mouse antidody
  misc_difference 715..717: note = n is a, c, g, or t
  misc_difference 796..798: note = n is a, c, g, or t
  misc_difference 892..900: note = n is a, c, g, or t
  misc_difference 982..984: note = n is a, c, g, or t
  misc_difference 988..990: note = n is a, c, g, or t
  source 1..1350: mol_type = other DNA
gaggtgcagctggtgcagagcggcgccgaggtgaagaagcccggcgccagcgtgaaggtgagctgcaaggccagcggctacaccttcaccacctacggcatgagctgggtgagacaggcccccggcaagggcctgcagtggatgggctggatcaacatctacagcggcatccccacctacgccgacgacttcaagggcagattcgccctgagcctggacaccagcaccagcaccgcctacatggagctgaacagcctgagagccgaggacaccgccgtgtactactgcgccagattcgacggccccgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcatggtgaccgtgcccagcagcagatggcccagcgagaccttcacctgcaacgtggcccaccccgccagcaagaccaaggtggacaagcccgtgcccaagagagagaacggcagagtgcccagaccccccgactgccccaagtgccccgcccccgagatgctgggcggcnnnagcgtgttcatcttcccccccaagcccaaggacaccctgctgatcgccagaacccccgaggtgacctgcgtggtggtgnnnctggaccccgaggaccccgaggtgcagatcagctggttcgtggacggcaagcagatgcagaccgccaagacccagcccagagaggagcagttcnnnnnnnnntacagagtggtgagcgtgctgcccatcggccaccaggactggctgaagggcaagcagttcacctgcaaggtgaacaacaagnnnctgnnnagccccatcgagagaaccatcagcaaggccagaggccaggcccaccagcccagcgtgtacgtgctgccccccagcagagaggagctgagcaagaacaccgtgagcctgacctgcctgatcaaggacttcttcccccccgacatcgacgtggagtggcagagcaacggccagcaggagcccgagagcaagtacagaaccacccccccccagctggacgaggacggcagctacttcctgtacagcaagctgagcgtggacaagagcagatggcagagaggcgacaccttcatctgcgccgtgatgcacgaggccctgcacaaccactacacccaggagagcctgagccacagccccggcaag

SEQ ID NO: 56 - protein, 450 aa, synthetic construct
  REGION 1..450: note = caninized mouse antibody
  VARIANT 239: note = misc_feature - Xaa can be any naturally occurring amino acid
  VARIANT 266: note = misc_feature - Xaa can be any naturally occurring amino acid
  VARIANT 298..300: note = misc_feature - Xaa can be any naturally occurring amino acid
  VARIANT 328: note = misc_feature - Xaa can be any naturally occurring amino acid
  VARIANT 330: note = misc_feature - Xaa can be any naturally occurring amino acid
EVQLVQSGAEVKKPGASVKVSCKASGYTFTTYGMSWVRQAPGKGLQWMGWINIYSGIPTYADDFKGRFALSLDTSTSTAYMELNSLRAEDTAVYYCARFDGPDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGXSVFIFPPKPKDTLLIARTPEVTCVVVXLDPEDPEVQISWFVDGKQMQTAKTQPREEQFXXXYRVVSVLPIGHQDWLKGKQFTCKVNNKXLXSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 57 - DNA, 1344 bp, synthetic construct
  misc_feature 1..1344: note = caninized mouse antibody
  misc_difference 709..711: note = n is a, c, g, or t
  misc_difference 790..792: note = n is a, c, g, or t
  misc_difference 886..894: note = n is a, c, g, or t
  misc_difference 976..978: note = n is a, c, g, or t
  misc_difference 982..984: note = n is a, c, g, or t
  source 1..1344: mol_type = other DNA
gaggtgcagctggtgcagagcggcgccgaggtgaagaagcccggcgccagcgtgaaggtgagctgcaaggccagcggctacaccttcaccacctacggcatgagctgggtgagacaggcccccggcaagggcctgcagtggatgggctggatcaacatctacagcggcatccccacctacgccgacgacttcaagggcagattcgccctgagcctggacaccagcaccagcaccgcctacatggagctgaacagcctgagagccgaggacaccgccgtgtactactgcgccagattcgacggccccgactactggggccagggcaccctggtgaccgtgagcagcgccagcaccaccgcccccagcgtgttccccctggcccccagctgcggcagccagagcggcagcaccgtggccctggcctgcctggtgagcggctacatccccgagcccgtgaccgtgagctggaacagcgtgagcctgaccagcggcgtgcacaccttccccagcgtgctgcagagcagcggcctgtacagcctgagcagcatggtgaccgtgcccagcagcagatggcccagcgagaccttcacctgcaacgtggcccaccccgccaccaacaccaaggtggacaagcccgtggccaaggagtgcgagtgcaagtgcaactgcaacaactgcccctgccccggctgcggcctgctgggcggcnnnagcgtgttcatcttcccccccaagcccaaggacatcctggtgaccgccagaacccccaccgtgacctgcgtggtggtgnnnctggaccccgagaaccccgaggtgcagatcagctggttcgtggacagcaagcaggtgcagaccgccaacacccagcccagagaggagcagagcnnnnnnnnntacagagtggtgagcgtgctgcccatcggccaccaggactggctgagcggcaagcagttcaagtgcaaggtgaacaacaagnnnctgnnnagccccatcgaggagatcatcagcaagacccccggccaggcccaccagcccaacgtgtacgtgctgccccccagcagagacgagatgagcaagaacaccgtgaccctgacctgcctggtgaaggacttcttcccccccgagatcgacgtggagtggcagagcaacggccagcaggagcccgagagcaagtacagaatgacccccccccagctggacgaggacggcagctacttcctgtacagcaagctgagcgtggacaagagcagatggcagagaggcgacaccttcatctgcgccgtgatgcacgaggccctgcacaaccactacacccagatcagcctgagccacagccccggcaag

SEQ ID NO: 58 - protein, 448 aa, synthetic construct
  REGION 1..448: note = caninized mouse antibody
  VARIANT 237: note = misc_feature - Xaa can be any naturally occurring amino acid
  VARIANT 264: note = misc_feature - Xaa can be any naturally occurring amino acid
  VARIANT 296..298: note = misc_feature - Xaa can be any naturally occurring amino acid
  VARIANT 326: note = misc_feature - Xaa can be any naturally occurring amino acid
  VARIANT 328: note = misc_feature - Xaa can be any naturally occurring amino acid
EVQLVQSGAEVKKPGASVKVSCKASGYTFTTYGMSWVRQAPGKGLQWMGWINIYSGIPTYADDFKGRFALSLDTSTSTAYMELNSLRAEDTAVYYCARFDGPDYWGQGTLVTVSSASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGXSVFIFPPKPKDILVTARTPTVTCVVVXLDPENPEVQISWFVDSKQVQTANTQPREEQSXXXYRVVSVLPIGHQDWLSGKQFKCKVNNKXLXSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 59 - DNA, 1365 bp, synthetic construct
  misc_feature 1..1365: note = caninized mouse antibody
  misc_difference 730..732: note = n is a, c, g, or t
  misc_difference 811..813: note = n is a, c, g, or t
  misc_difference 907..915: note = n is a, c, g, or t
  misc_difference 997..999: note = n is a, c, g, or t
  misc_difference 1003..1005: note = n is a, c, g, or t
  source 1..1365: mol_type = other DNA
gaggtgcagctggtgcagagcgtggccgagctggtgaagcccggcgccagcgtgaaggtgagctgcaccgtgagcggcttcaacatcaagaacacctacatgcactgggtgagacaggcccccggcaagggcctgcagtggatcggcagaatcgaccccgccaacgtgaacaccaagtacgcccccaagttccagggcagagccaccatcaccgccgacaccagcaccaacaccgcctacatgcagctgagcagcctgagagccgaggacaccgccgtgtactactgcgccagaatctactacgactacgacggcgacatcgacgtgtggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcatggtgaccgtgcccagcagcagatggcccagcgagaccttcacctgcaacgtggcccaccccgccagcaagaccaaggtggacaagcccgtgcccaagagagagaacggcagagtgcccagaccccccgactgccccaagtgccccgcccccgagatgctgggcggcnnnagcgtgttcatcttcccccccaagcccaaggacaccctgctgatcgccagaacccccgaggtgacctgcgtggtggtgnnnctggaccccgaggaccccgaggtgcagatcagctggttcgtggacggcaagcagatgcagaccgccaagacccagcccagagaggagcagttcnnnnnnnnntacagagtggtgagcgtgctgcccatcggccaccaggactggctgaagggcaagcagttcacctgcaaggtgaacaacaagnnnctgnnnagccccatcgagagaaccatcagcaaggccagaggccaggcccaccagcccagcgtgtacgtgctgccccccagcagagaggagctgagcaagaacaccgtgagcctgacctgcctgatcaaggacttcttcccccccgacatcgacgtggagtggcagagcaacggccagcaggagcccgagagcaagtacagaaccacccccccccagctggacgaggacggcagctacttcctgtacagcaagctgagcgtggacaagagcagatggcagagaggcgacaccttcatctgcgccgtgatgcacgaggccctgcacaaccactacacccaggagagcctgagccacagccccggcaag

SEQ ID NO: 60 - protein, 455 aa, synthetic construct
  REGION 1..455: note = caninized mouse antibody
  VARIANT 244: note = misc_feature - Xaa can be any naturally occurring amino acid
  VARIANT 271: note = misc_feature - Xaa can be any naturally occurring amino acid
  VARIANT 303..305: note = misc_feature - Xaa can be any naturally occurring amino acid
  VARIANT 333: note = misc_feature - Xaa can be any naturally occurring amino acid
  VARIANT 335: note = misc_feature - Xaa can be any naturally occurring amino acid
EVQLVQSVAELVKPGASVKVSCTVSGFNIKNTYMHWVRQAPGKGLQWIGRIDPANVNTKYAPKFQGRATITADTSTNTAYMQLSSLRAEDTAVYYCARIYYDYDGDIDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGXSVFIFPPKPKDTLLIARTPEVTCVVVXLDPEDPEVQISWFVDGKQMQTAKTQPREEQFXXXYRVVSVLPIGHQDWLKGKQFTCKVNNKXLXSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 61 - DNA, 1359 bp, synthetic construct
  misc_feature 1..1359: note = caninized mouse antibody
  misc_difference 724..726: note = n is a, c, g, or t
  misc_difference 805..807: note = n is a, c, g, or t
  misc_difference 901..909: note = n is a, c, g, or t
  misc_difference 991..993: note = n is a, c, g, or t
  misc_difference 997..999: note = n is a, c, g, or t
  source 1..1359: mol_type = other DNA
gaggtgcagctggtgcagagcgtggccgagctggtgaagcccggcgccagcgtgaaggtgagctgcaccgtgagcggcttcaacatcaagaacacctacatgcactgggtgagacaggcccccggcaagggcctgcagtggatcggcagaatcgaccccgccaacgtgaacaccaagtacgcccccaagttccagggcagagccaccatcaccgccgacaccagcaccaacaccgcctacatgcagctgagcagcctgagagccgaggacaccgccgtgtactactgcgccagaatctactacgactacgacggcgacatcgacgtgtggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcatggtgaccgtgcccagcagcagatggcccagcgagaccttcacctgcaacgtggcccaccccgccagcaagaccaaggtggacaagcccgtggccaaggagtgcgagtgcaagtgcaactgcaacaactgcccctgccccggctgcggcctgctgggcggcnnnagcgtgttcatcttcccccccaagcccaaggacatcctggtgaccgccagaacccccaccgtgacctgcgtggtggtgnnnctggaccccgagaaccccgaggtgcagatcagctggttcgtggacagcaagcaggtgcagaccgccaacacccagcccagagaggagcagagcnnnnnnnnntacagagtggtgagcgtgctgcccatcggccaccaggactggctgagcggcaagcagttcaagtgcaaggtgaacaacaagnnnctgnnnagccccatcgaggagatcatcagcaagacccccggccaggcccaccagcccaacgtgtacgtgctgccccccagcagagacgagatgagcaagaacaccgtgaccctgacctgcctggtgaaggacttcttcccccccgagatcgacgtggagtggcagagcaacggccagcaggagcccgagagcaagtacagaatgacccccccccagctggacgaggacggcagctacttcctgtacagcaagctgagcgtggacaagagcagatggcagagaggcgacaccttcatctgcgccgtgatgcacgaggccctgcacaaccactacacccagatcagcctgagccacagccccggcaag

SEQ ID NO: 62 - protein, 453 aa, synthetic construct
  REGION 1..453: note = caninized mouse antibody
  VARIANT 242: note = misc_feature - Xaa can be any naturally occurring amino acid
  VARIANT 269: note = misc_feature - Xaa can be any naturally occurring amino acid
  VARIANT 301..303: note = misc_feature - Xaa can be any naturally occurring amino acid
  VARIANT 331: note = misc_feature - Xaa can be any naturally occurring amino acid
  VARIANT 333: note = misc_feature - Xaa can be any naturally occurring amino acid
EVQLVQSVAELVKPGASVKVSCTVSGFNIKNTYMHWVRQAPGKGLQWIGRIDPANVNTKYAPKFQGRATITADTSTNTAYMQLSSLRAEDTAVYYCARIYYDYDGDIDVWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVAKECECKCNCNNCPCPGCGLLGGXSVFIFPPKPKDILVTARTPTVTCVVVXLDPENPEVQISWFVDSKQVQTANTQPREEQSXXXYRVVSVLPIGHQDWLSGKQFKCKVNNKXLXSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 63 - DNA, 1350 bp, synthetic construct
  misc_feature 1..1350: note = caninized mouse antibody
  misc_difference 715..717: note = n is a, c, g, or t
  misc_difference 796..798: note = n is a, c, g, or t
  misc_difference 892..900: note = n is a, c, g, or t
  misc_difference 982..984: note = n is a, c, g, or t
  misc_difference 988..990: note = n is a, c, g, or t
  source 1..1350: mol_type = other DNA
gaggtgcagctggtgcagagcggcgccgaggtgaagaagcccggcgccagcgtgaaggtgagctgcaaggccagcggctacaccttcaccacctacggcatgagctgggtgagacaggcccccggcaagggcctgcagtggatgggctggatcaacatctacagcggcatgcccacctacgccgacgacttcaagggcagattcgccctgagcctggacaccagcaccagcaccgcctacatggagctgaacagcctgagagccgaggacaccgccgtgtactactgcaccagattcgacggccccgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcatggtgaccgtgcccagcagcagatggcccagcgagaccttcacctgcaacgtggcccaccccgccagcaagaccaaggtggacaagcccgtgcccaagagagagaacggcagagtgcccagaccccccgactgccccaagtgccccgcccccgagatgctgggcggcnnnagcgtgttcatcttcccccccaagcccaaggacaccctgctgatcgccagaacccccgaggtgacctgcgtggtggtgnnnctggaccccgaggaccccgaggtgcagatcagctggttcgtggacggcaagcagatgcagaccgccaagacccagcccagagaggagcagttcnnnnnnnnntacagagtggtgagcgtgctgcccatcggccaccaggactggctgaagggcaagcagttcacctgcaaggtgaacaacaagnnnctgnnnagccccatcgagagaaccatcagcaaggccagaggccaggcccaccagcccagcgtgtacgtgctgccccccagcagagaggagctgagcaagaacaccgtgagcctgacctgcctgatcaaggacttcttcccccccgacatcgacgtggagtggcagagcaacggccagcaggagcccgagagcaagtacagaaccacccccccccagctggacgaggacggcagctacttcctgtacagcaagctgagcgtggacaagagcagatggcagagaggcgacaccttcatctgcgccgtgatgcacgaggccctgcacaaccactacacccaggagagcctgagccacagccccggcaag

SEQ ID NO: 64 - protein, 450 aa, synthetic construct
  REGION 1..450: note = caninized mouse antibody
  VARIANT 239: note = misc_feature - Xaa can be any naturally occurring amino acid
  VARIANT 266: note = misc_feature - Xaa can be any naturally occurring amino acid
  VARIANT 298..300: note = misc_feature - Xaa can be any naturally occurring amino acid
  VARIANT 328: note = misc_feature - Xaa can be any naturally occurring amino acid
  VARIANT 330: note = misc_feature - Xaa can be any naturally occurring amino acid
EVQLVQSGAEVKKPGASVKVSCKASGYTFTTYGMSWVRQAPGKGLQWMGWINIYSGMPTYADDFKGRFALSLDTSTSTAYMELNSLRAEDTAVYYCTRFDGPDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGXSVFIFPPKPKDTLLIARTPEVTCVVVXLDPEDPEVQISWFVDGKQMQTAKTQPREEQFXXXYRVVSVLPIGHQDWLKGKQFTCKVNNKXLXSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 65 - DNA, 1344 bp, synthetic construct
  misc_feature 1..1344: note = caninized mouse antibody
  misc_difference 709..711: note = n is a, c, g, or t
  misc_difference 790..792: note = n is a, c, g, or t
  misc_difference 886..894: note = n is a, c, g, or t
  misc_difference 976..978: note = n is a, c, g, or t
  misc_difference 982..984: note = n is a, c, g, or t
  source 1..1344: mol_type = other DNA
gaggtgcagctggtgcagagcggcgccgaggtgaagaagcccggcgccagcgtgaaggtgagctgcaaggccagcggctacaccttcaccacctacggcatgagctgggtgagacaggcccccggcaagggcctgcagtggatgggctggatcaacatctacagcggcatgcccacctacgccgacgacttcaagggcagattcgccctgagcctggacaccagcaccagcaccgcctacatggagctgaacagcctgagagccgaggacaccgccgtgtactactgcaccagattcgacggccccgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcatggtgaccgtgcccagcagcagatggcccagcgagaccttcacctgcaacgtggcccaccccgccagcaagaccaaggtggacaagcccgtggccaaggagtgcgagtgcaagtgcaactgcaacaactgcccctgccccggctgcggcctgctgggcggcnnnagcgtgttcatcttcccccccaagcccaaggacatcctggtgaccgccagaacccccaccgtgacctgcgtggtggtgnnnctggaccccgagaaccccgaggtgcagatcagctggttcgtggacagcaagcaggtgcagaccgccaacacccagcccagagaggagcagagcnnnnnnnnntacagagtggtgagcgtgctgcccatcggccaccaggactggctgagcggcaagcagttcaagtgcaaggtgaacaacaagnnnctgnnnagccccatcgaggagatcatcagcaagacccccggccaggcccaccagcccaacgtgtacgtgctgccccccagcagagacgagatgagcaagaacaccgtgaccctgacctgcctggtgaaggacttcttcccccccgagatcgacgtggagtggcagagcaacggccagcaggagcccgagagcaagtacagaatgacccccccccagctggacgaggacggcagctacttcctgtacagcaagctgagcgtggacaagagcagatggcagagaggcgacaccttcatctgcgccgtgatgcacgaggccctgcacaaccactacacccagatcagcctgagccacagccccggcaag

SEQ ID NO: 66 - protein, 448 aa, synthetic construct
  REGION 1..448: note = caninized mouse antibody
  VARIANT 237: note = misc_feature - Xaa can be any naturally occurring amino acid
  VARIANT 264: note = misc_feature - Xaa can be any naturally occurring amino acid
  VARIANT 296..298: note = misc_feature - Xaa can be any naturally occurring amino acid
  VARIANT 326: note = misc_feature - Xaa can be any naturally occurring amino acid
  VARIANT 328: note = misc_feature - Xaa can be any naturally occurring amino acid
EVQLVQSGAEVKKPGASVKVSCKASGYTFTTYGMSWVRQAPGKGLQWMGWINIYSGMPTYADDFKGRFALSLDTSTSTAYMELNSLRAEDTAVYYCTRFDGPDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVAKECECKCNCNNCPCPGCGLLGGXSVFIFPPKPKDILVTARTPTVTCVVVXLDPENPEVQISWFVDSKQVQTANTQPREEQSXXXYRVVSVLPIGHQDWLSGKQFKCKVNNKXLXSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 67 - DNA, 1338 bp, synthetic construct
  misc_feature 1..1338: note = caninized mouse antibody
  source 1..1338: mol_type = other DNA
gaggtgcagctggtgcagagcggcggcgacctggtgaagcccggcggcagcgtgagactgagctgcgtggccagcggctacaccttcaccacctacggcatgagctgggtgagacaggcccccggcaagggcctgcagtggatgggctggatcaacatctacagcggcatccccacctacgccgacgacttcaagggcagattcaccttcagcctggacaccgccaagaacaccgcctacctgcagctgaacagcctgagagccgaggacaccgccgtgtactactgcaccagattcgacggccccgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgcacagcctgagcagcatggtgaccgtgcccagcagcagatggcccagcgagaccttcacctgcaacgtggtgcaccccgccagcaacaccaaggtggacaagcccgtgttcaacgagtgcagatgcaccgacacccccccctgccccgtgcccgagcccctgggcggccccagcgtgctgatcttcccccccaagcccaaggacatcctgagaatcaccagaacccccgaggtgacctgcgtggtgctggacctgggcagagaggaccccgaggtgcagatcagctggttcgtggacggcaaggaggtgcacaccgccaagacccagagcagagagcagcagttcaacggcacctacagagtggtgagcgtgctgcccatcgagcaccaggactggctgaccggcaaggagttcaagtgcagagtgaaccacatcgacctgcccagccccatcgagagaaccatcagcaaggccagaggcagagcccacaagcccagcgtgtacgtgctgccccccagccccaaggagctgagcagcagcgacaccgtgagcatcacctgcctgatcaaggacttctacccccccgacatcgacgtggagtggcagagcaacggccagcaggagcccgagagaaagcacagaatgacccccccccagctggacgaggacggcagctacttcctgtacagcaagctgagcgtggacaagagcagatggcagcagggcgaccccttcacctgcgccgtgatgcacgagaccctgcagaaccactacaccgacctgagcctgagccacagccccggcaag

SEQ ID NO: 68 - protein, 446 aa, synthetic construct
  REGION 1..446: note = caninized mouse antibody
EVQLVQSGGDLVKPGGSVRLSCVASGYTFTTYGMSWVRQAPGKGLQWMGWINIYSGIPTYADDFKGRFTFSLDTAKNTAYLQLNSLRAEDTAVYYCTRFDGPDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 69 - DNA, 1338 bp, synthetic construct
  misc_feature 1..1338: note = caninized mouse antibody
  source 1..1338: mol_type = other DNA
gaggtgcagctggtgcagagcggcggcgacctggtgaagcccggcggcagcgtgagactgagctgcgtggccagcggctacaccttcaccacctacggcatgagctgggtgagacaggcccccggcaagggcctgcagtggatgggctggatcaacatctacagcggcatccccacctacgccgacgacttcaagggcagattcaccttcagcctggacaccgccaagaacaccgcctacctgcagctgaacagcctgagagccgaggacaccgccgtgtactactgcaccagattcgacggccccgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcaccgtgaccgtgcccagcagcagatggcccagcgagaccttcacctgcaacgtggtgcaccccgccagcaacaccaaggtggacaagcccgtgcccaaggagagcacctgcaagtgcatcagcccctgccccgtgcccgagagcctgggcggccccagcgtgttcatcttcccccccaagcccaaggacatcctgagaatcaccagaacccccgagatcacctgcgtggtgctggacctgggcagagaggaccccgaggtgcagatcagctggttcgtggacggcaaggaggtgcacaccgccaagacccagcccagagagcagcagttcaacagcacctacagagtggtgagcgtgctgcccatcgagcaccaggactggctgaccggcaaggagttcaagtgcagagtgaaccacatcggcctgcccagccccatcgagagaaccatcagcaaggccagaggccaggcccaccagcccagcgtgtacgtgctgccccccagccccaaggagctgagcagcagcgacaccgtgaccctgacctgcctgatcaaggacttcttcccccccgagatcgacgtggagtggcagagcaacggccagcccgagcccgagagcaagtaccacaccaccgccccccagctggacgaggacggcagctacttcctgtacagcaagctgagcgtggacaagagcagatggcagcagggcgacaccttcacctgcgccgtgatgcacgaggccctgcagaaccactacaccgacctgagcctgagccacagccccggcaag

SEQ ID NO: 70 - protein, 446 aa, synthetic construct
  REGION 1..446: note = caninized mouse antibody
EVQLVQSGGDLVKPGGSVRLSCVASGYTFTTYGMSWVRQAPGKGLQWMGWINIYSGIPTYADDFKGRFTFSLDTAKNTAYLQLNSLRAEDTAVYYCTRFDGPDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 71 - DNA, 672 bp, synthetic construct
  misc_feature 1..672: note = caninized mouse antibody
  source 1..672: mol_type = other DNA
gacatcgtgatgacccagacccccctgagcctgagcgtgagccccggcgagcccgccagcatgagctgcaagagcagccagagcctgctgaacagcgtgaaccagaagaactacctggcctggtacagacagaagcccggccagagcccccaggtgctggtgtacttcgccagcgccagagtgagcggcgtgcccgacagattcatcggcagcggcagcggcaccgacttcaccctgagaatcagcagagtggaggccgacgacctgggcgtgtactactgccagcagtacttcagcacccccctgaccttcggccagggcaccaagctggagctgaagagaaacgacgcccagcccgccgtgtacctgttccagcccagccccgaccagctgcacaccggcagcgccagcgtggtgtgcctgctgaacagcttctaccccaaggacatcaacgtgaagtggaaggtggacggcgtgatccaggacaccggcatccaggagagcgtgaccgagcaggacagcaaggacagcacctacagcctgagcagcaccctgaccatgagcagcaccgagtacctgagccacgagctgtacagctgcgagatcacccacaagagcctgcccagcaccctgatcaagagcttccagagaagcgagtgccagagagtggac

SEQ ID NO: 72 - protein, 224 aa, synthetic construct
  REGION 1..224: note = caninized mouse antibody
DIVMTQTPLSLSVSPGEPASMSCKSSQSLLNSVNQKNYLAWYRQKPGQSPQVLVYFASARVSGVPDRFIGSGSGTDFTLRISRVEADDLGVYYCQQYFSTPLTFGQGTKLELKRNDAQPAVYLFQPSPDQLHTGSASVVCLLNSFYPKDINVKWKVDGVIQDTGIQESVTEQDSKDSTYSLSSTLTMSSTEYLSHELYSCEITHKSLPSTLIKSFQRSECQRVD

SEQ ID NO: 73 - DNA, 1353 bp, synthetic construct
  misc_feature 1..1353: note = caninized mouse antibody
  source 1..1353: mol_type = other DNA
gaggtgcagctggtgcagagcggcggcgacctggtgaagcccggcggcagcgtgagactgagctgcgtggccagcggcttcaacatcaagaacacctacatgcactgggtgagacaggcccccggcaagggcctgcagtggatcggcagaatcgcccccgccaacgtggacaccaagtacgcccccaagttccagggcaaggccaccatcagcgccgacaccgccaagaacaccgcctacatgcagctgaacagcctgagagccgaggacaccgccgtgtactactgcgtgctgatctactacgactacgacggcgacatcgacgtgtggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgcacagcctgagcagcatggtgaccgtgcccagcagcagatggcccagcgagaccttcacctgcaacgtggtgcaccccgccagcaacaccaaggtggacaagcccgtgttcaacgagtgcagatgcaccgacacccccccctgccccgtgcccgagcccctgggcggccccagcgtgctgatcttcccccccaagcccaaggacatcctgagaatcaccagaacccccgaggtgacctgcgtggtgctggacctgggcagagaggaccccgaggtgcagatcagctggttcgtggacggcaaggaggtgcacaccgccaagacccagagcagagagcagcagttcaacggcacctacagagtggtgagcgtgctgcccatcgagcaccaggactggctgaccggcaaggagttcaagtgcagagtgaaccacatcgacctgcccagccccatcgagagaaccatcagcaaggccagaggcagagcccacaagcccagcgtgtacgtgctgccccccagccccaaggagctgagcagcagcgacaccgtgagcatcacctgcctgatcaaggacttctacccccccgacatcgacgtggagtggcagagcaacggccagcaggagcccgagagaaagcacagaatgacccccccccagctggacgaggacggcagctacttcctgtacagcaagctgagcgtggacaagagcagatggcagcagggcgaccccttcacctgcgccgtgatgcacgagaccctgcagaaccactacaccgacctgagcctgagccacagccccggcaag

SEQ ID NO: 74 - protein, 451 aa, synthetic construct
  REGION 1..451: note = caninized mouse antibody
EVQLVQSGGDLVKPGGSVRLSCVASGFNIKNTYMHWVRQAPGKGLQWIGRIAPANVDTKYAPKFQGKATISADTAKNTAYMQLNSLRAEDTAVYYCVLIYYDYDGDIDVWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 75 - DNA, 1353 bp, synthetic construct
  misc_feature 1..1353: note = caninized mouse antibody
  source 1..1353: mol_type = other DNA
gaggtgcagctggtgcagagcggcggcgacctggtgaagcccggcggcagcgtgagactgagctgcgtggccagcggcttcaacatcaagaacacctacatgcactgggtgagacaggcccccggcaagggcctgcagtggatcggcagaatcgcccccgccaacgtggacaccaagtacgcccccaagttccagggcaaggccaccatcagcgccgacaccgccaagaacaccgcctacatgcagctgaacagcctgagagccgaggacaccgccgtgtactactgcgtgctgatctactacgactacgacggcgacatcgacgtgtggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcaccgtgaccgtgcccagcagcagatggcccagcgagaccttcacctgcaacgtggtgcaccccgccagcaacaccaaggtggacaagcccgtgcccaaggagagcacctgcaagtgcatcagcccctgccccgtgcccgagagcctgggcggccccagcgtgttcatcttcccccccaagcccaaggacatcctgagaatcaccagaacccccgagatcacctgcgtggtgctggacctgggcagagaggaccccgaggtgcagatcagctggttcgtggacggcaaggaggtgcacaccgccaagacccagcccagagagcagcagttcaacagcacctacagagtggtgagcgtgctgcccatcgagcaccaggactggctgaccggcaaggagttcaagtgcagagtgaaccacatcggcctgcccagccccatcgagagaaccatcagcaaggccagaggccaggcccaccagcccagcgtgtacgtgctgccccccagccccaaggagctgagcagcagcgacaccgtgaccctgacctgcctgatcaaggacttcttcccccccgagatcgacgtggagtggcagagcaacggccagcccgagcccgagagcaagtaccacaccaccgccccccagctggacgaggacggcagctacttcctgtacagcaagctgagcgtggacaagagcagatggcagcagggcgacaccttcacctgcgccgtgatgcacgaggccctgcagaaccactacaccgacctgagcctgagccacagccccggcaag

SEQ ID NO: 76 - protein, 451 aa, synthetic construct
  REGION 1..451: note = caninized mouse antibody
EVQLVQSGGDLVKPGGSVRLSCVASGFNIKNTYMHWVRQAPGKGLQWIGRIAPANVDTKYAPKFQGKATISADTAKNTAYMQLNSLRAEDTAVYYCVLIYYDYDGDIDVWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 77 - DNA, 654 bp, synthetic construct
  misc_feature 1..654: note = caninized mouse antibody
  source 1..654: mol_type = other DNA
gacatcgtgatgacccagacccccctgagcctgagcgtgagcctgggcgagcccgccagcatcagctgccacgccagccagaacatcaacgtgtggctgagctggtacagacagaagcccggccagatcccccagctgctgatctacaaggccagccacctgcacaccggcgtgcccgacagattcagcggcagcggcagcggcaccgacttcaccctgagaatcagcagagtggaggccgacgacgccggcgtgtactactgccagcagggccagagctggcccctgaccttcggccagggcaccaaggtggagatcaagagaaacgacgcccagcccgccgtgtacctgttccagcccagccccgaccagctgcacaccggcagcgccagcgtggtgtgcctgctgaacagcttctaccccaaggacatcaacgtgaagtggaaggtggacggcgtgatccaggacaccggcatccaggagagcgtgaccgagcaggacagcaaggacagcacctacagcctgagcagcaccctgaccatgagcagcaccgagtacctgagccacgagctgtacagctgcgagatcacccacaagagcctgcccagcaccctgatcaagagcttccagagaagcgagtgccagagagtggac

SEQ ID NO: 78 - protein, 218 aa, synthetic construct
  REGION 1..218: note = caninized mouse antibody
DIVMTQTPLSLSVSLGEPASISCHASQNINVWLSWYRQKPGQIPQLLIYKASHLHTGVPDRFSGSGSGTDFTLRISRVEADDAGVYYCQQGQSWPLTFGQGTKVEIKRNDAQPAVYLFQPSPDQLHTGSASVVCLLNSFYPKDINVKWKVDGVIQDTGIQESVTEQDSKDSTYSLSSTLTMSSTEYLSHELYSCEITHKSLPSTLIKSFQRSECQRVD

SEQ ID NO: 79 - DNA, 1359 bp, synthetic construct
  misc_feature 1..1359: note = caninized mouse antibody
  source 1..1359: mol_type = other DNA
gaggtgcagctggtgcagagcggcggcgacctggtgaagcccggcggcagcgtgagactgagctgcgtggccagcggctacaccttcaccagatacaacatgcactgggtgagacaggcccccggcaagggcctgcagtggatcggcaccatctaccccggctacggcgacaccagctacaaccagaagttcaagggcaaggccaccctgagcgtggacatcgccaagaacaccgcctacatgcagctgaacagcctgagagccgaggacaccgccgtgtacttctgcagcagagagttcgccgacgactaccccatcccccccttcgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgcacagcctgagcagcatggtgaccgtgcccagcagcagatggcccagcgagaccttcacctgcaacgtggtgcaccccgccagcaacaccaaggtggacaagcccgtgttcaacgagtgcagatgcaccgacacccccccctgccccgtgcccgagcccctgggcggccccagcgtgctgatcttcccccccaagcccaaggacatcctgagaatcaccagaacccccgaggtgacctgcgtggtgctggacctgggcagagaggaccccgaggtgcagatcagctggttcgtggacggcaaggaggtgcacaccgccaagacccagagcagagagcagcagttcaacggcacctacagagtggtgagcgtgctgcccatcgagcaccaggactggctgaccggcaaggagttcaagtgcagagtgaaccacatcgacctgcccagccccatcgagagaaccatcagcaaggccagaggcagagcccacaagcccagcgtgtacgtgctgccccccagccccaaggagctgagcagcagcgacaccgtgagcatcacctgcctgatcaaggacttctacccccccgacatcgacgtggagtggcagagcaacggccagcaggagcccgagagaaagcacagaatgacccccccccagctggacgaggacggcagctacttcctgtacagcaagctgagcgtggacaagagcagatggcagcagggcgaccccttcacctgcgccgtgatgcacgagaccctgcagaaccactacaccgacctgagcctgagccacagccccggcaag

SEQ ID NO: 80 - protein, 453 aa, synthetic construct
  REGION 1..453: note = caninized mouse antibody
EVQLVQSGGDLVKPGGSVRLSCVASGYTFTRYNMHWVRQAPGKGLQWIGTIYPGYGDTSYNQKFKGKATLSVDIAKNTAYMQLNSLRAEDTAVYFCSREFADDYPIPPFDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 81 - DNA, 1359 bp, synthetic construct
  misc_feature 1..1359: note = caninized mouse antibody
  source 1..1359: mol_type = other DNA
gaggtgcagctggtgcagagcggcggcgacctggtgaagcccggcggcagcgtgagactgagctgcgtggccagcggctacaccttcaccagatacaacatgcactgggtgagacaggcccccggcaagggcctgcagtggatcggcaccatctaccccggctacggcgacaccagctacaaccagaagttcaagggcaaggccaccctgagcgtggacatcgccaagaacaccgcctacatgcagctgaacagcctgagagccgaggacaccgccgtgtacttctgcagcagagagttcgccgacgactaccccatcccccccttcgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcaccgtgaccgtgcccagcagcagatggcccagcgagaccttcacctgcaacgtggtgcaccccgccagcaacaccaaggtggacaagcccgtgcccaaggagagcacctgcaagtgcatcagcccctgccccgtgcccgagagcctgggcggccccagcgtgttcatcttcccccccaagcccaaggacatcctgagaatcaccagaacccccgagatcacctgcgtggtgctggacctgggcagagaggaccccgaggtgcagatcagctggttcgtggacggcaaggaggtgcacaccgccaagacccagcccagagagcagcagttcaacagcacctacagagtggtgagcgtgctgcccatcgagcaccaggactggctgaccggcaaggagttcaagtgcagagtgaaccacatcggcctgcccagccccatcgagagaaccatcagcaaggccagaggccaggcccaccagcccagcgtgtacgtgctgccccccagccccaaggagctgagcagcagcgacaccgtgaccctgacctgcctgatcaaggacttcttcccccccgagatcgacgtggagtggcagagcaacggccagcccgagcccgagagcaagtaccacaccaccgccccccagctggacgaggacggcagctacttcctgtacagcaagctgagcgtggacaagagcagatggcagcagggcgacaccttcacctgcgccgtgatgcacgaggccctgcagaaccactacaccgacctgagcctgagccacagccccggcaag

SEQ ID NO: 82 - protein, 453 aa, synthetic construct
  REGION 1..453: note = caninized mouse antibody
EVQLVQSGGDLVKPGGSVRLSCVASGYTFTRYNMHWVRQAPGKGLQWIGTIYPGYGDTSYNQKFKGKATLSVDIAKNTAYMQLNSLRAEDTAVYFCSREFADDYPIPPFDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 83 - DNA, 666 bp, synthetic construct
  misc_feature 1..666: note = caninized mouse antibody
  source 1..666: mol_type = other DNA
gacatcgtgatgacccagacccccctgagcctgcccgtgagcctgggcgagcccgccagcatcagctgcagaagcagccagaacatcgtgcacagcaacggcaacacctacctggagtggtacagacagaagcccggccagagcccccagctgctgatctacaaggtgagcaacagattcagcggcgtgcccgacagattcagcggcagcggcagcggcaccgacttcaccctgagaatcagcagagtggaggccgacgacgccggcgtgtactactgcttccagggcagccacgtgccctacaccttcggccagggcaccaaggtggagatcaagagagacgcccagcccgccgtgtacctgttccagcccagccccgaccagctgcacaccggcagcgccagcgtggtgtgcctgctgaacagcttctaccccaaggacatcaacgtgaagtggaaggtggacggcgtgatccaggacaccggcatccaggagagcgtgaccgagcaggacagcaaggacagcacctacagcctgagcagcaccctgaccatgagcagcaccgagtacctgagccacgagctgtacagctgcgagatcacccacaagagcctgcccagcaccctgatcaagagcttccagagaagcgagtgccagagagtggac

SEQ ID NO: 84 - protein, 222 aa, synthetic construct
  REGION 1..222: note = caninized mouse antibody
DIVMTQTPLSLPVSLGEPASISCRSSQNIVHSNGNTYLEWYRQKPGQSPQLLIYKVSNRFSGVPDRFSGSGSGTDFTLRISRVEADDAGVYYCFQGSHVPYTFGQGTKVEIKRDAQPAVYLFQPSPDQLHTGSASVVCLLNSFYPKDINVKWKVDGVIQDTGIQESVTEQDSKDSTYSLSSTLTMSSTEYLSHELYSCEITHKSLPSTLIKSFQRSECQRVD

SEQ ID NO: 85 - DNA, 1338 bp, synthetic construct
  misc_feature 1..1338: note = caninized mouse antibody
  source 1..1338: mol_type = other DNA
gaggtgcagctggtgcagagcggccccggcctggtgaagcccagccagagcctgagcctgacctgcgtggtgagcggcttcagcctgaccagctacggcgtgcactgggtgagacaggcccccggcaagggcctgcagtggatgggctggatcaacatctacagcggcatccccacctacgccgacgacttcaagggcagattcaccttcagcctggacaccgccaagaacaccgcctacctgcagctgagcagcctgagagccgaggacaccgccgtgtactactgcgccagattcgacggccccgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgcacagcctgagcagcatggtgaccgtgcccagcagcagatggcccagcgagaccttcacctgcaacgtggtgcaccccgccagcaacaccaaggtggacaagcccgtgttcaacgagtgcagatgcaccgacacccccccctgccccgtgcccgagcccctgggcggccccagcgtgctgatcttcccccccaagcccaaggacatcctgagaatcaccagaacccccgaggtgacctgcgtggtgctggacctgggcagagaggaccccgaggtgcagatcagctggttcgtggacggcaaggaggtgcacaccgccaagacccagagcagagagcagcagttcaacggcacctacagagtggtgagcgtgctgcccatcgagcaccaggactggctgaccggcaaggagttcaagtgcagagtgaaccacatcgacctgcccagccccatcgagagaaccatcagcaaggccagaggcagagcccacaagcccagcgtgtacgtgctgccccccagccccaaggagctgagcagcagcgacaccgtgagcatcacctgcctgatcaaggacttctacccccccgacatcgacgtggagtggcagagcaacggccagcaggagcccgagagaaagcacagaatgacccccccccagctggacgaggacggcagctacttcctgtacagcaagctgagcgtggacaagagcagatggcagcagggcgaccccttcacctgcgccgtgatgcacgagaccctgcagaaccactacaccgacctgagcctgagccacagccccggcaag

SEQ ID NO: 86 - protein, 446 aa, synthetic construct
  REGION 1..446: note = caninized mouse antibody
EVQLVQSGPGLVKPSQSLSLTCVVSGFSLTSYGVHWVRQAPGKGLQWMGWINIYSGIPTYADDFKGRFTFSLDTAKNTAYLQLSSLRAEDTAVYYCARFDGPDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 87 - DNA, 1338 bp, synthetic construct
  misc_feature 1..1338: note = caninized mouse antibody
  source 1..1338: mol_type = other DNA
gaggtgcagctggtgcagagcggccccggcctggtgaagcccagccagagcctgagcctgacctgcgtggtgagcggcttcagcctgaccagctacggcgtgcactgggtgagacaggcccccggcaagggcctgcagtggatgggctggatcaacatctacagcggcatccccacctacgccgacgacttcaagggcagattcaccttcagcctggacaccgccaagaacaccgcctacctgcagctgagcagcctgagagccgaggacaccgccgtgtactactgcgccagattcgacggccccgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcaccgtgaccgtgcccagcagcagatggcccagcgagaccttcacctgcaacgtggtgcaccccgccagcaacaccaaggtggacaagcccgtgcccaaggagagcacctgcaagtgcatcagcccctgccccgtgcccgagagcctgggcggccccagcgtgttcatcttcccccccaagcccaaggacatcctgagaatcaccagaacccccgagatcacctgcgtggtgctggacctgggcagagaggaccccgaggtgcagatcagctggttcgtggacggcaaggaggtgcacaccgccaagacccagcccagagagcagcagttcaacagcacctacagagtggtgagcgtgctgcccatcgagcaccaggactggctgaccggcaaggagttcaagtgcagagtgaaccacatcggcctgcccagccccatcgagagaaccatcagcaaggccagaggccaggcccaccagcccagcgtgtacgtgctgccccccagccccaaggagctgagcagcagcgacaccgtgaccctgacctgcctgatcaaggacttcttcccccccgagatcgacgtggagtggcagagcaacggccagcccgagcccgagagcaagtaccacaccaccgccccccagctggacgaggacggcagctacttcctgtacagcaagctgagcgtggacaagagcagatggcagcagggcgacaccttcacctgcgccgtgatgcacgaggccctgcagaaccactacaccgacctgagcctgagccacagccccggcaag

SEQ ID NO: 88 - protein, 446 aa, synthetic construct
  REGION 1..446: note = caninized mouse antibody
EVQLVQSGPGLVKPSQSLSLTCVVSGFSLTSYGVHWVRQAPGKGLQWMGWINIYSGIPTYADDFKGRFTFSLDTAKNTAYLQLSSLRAEDTAVYYCARFDGPDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 89 - DNA, 672 bp, synthetic construct
  misc_feature 1..672: note = caninized mouse antibody
  source 1..672: mol_type = other DNA
gacatcgtgatgacccagacccccctgagcctgagcgtgagccccggcgagcccgccagcatgagctgcaagagcagccagagcctgctgaacagcgtgaaccagaagaactacctggcctggtacagacagaagcccggccagagcccccaggtgctggtgtacttcgccagcaccagagtgagcggcgtgcccgacagattcatcggcagcggcagcggcaccgacttcaccctgagaatcagcagagtggaggccgacgacctgggcgtgtactactgccagcagtacttcagcacccccctgaccttcggccagggcaccaagctggagctgaagagaaacgacgcccagcccgccgtgtacctgttccagcccagccccgaccagctgcacaccggcagcgccagcgtggtgtgcctgctgaacagcttctaccccaaggacatcaacgtgaagtggaaggtggacggcgtgatccaggacaccggcatccaggagagcgtgaccgagcaggacagcaaggacagcacctacagcctgagcagcaccctgaccatgagcagcaccgagtacctgagccacgagctgtacagctgcgagatcacccacaagagcctgcccagcaccctgatcaagagcttccagagaagcgagtgccagagagtggac

SEQ ID NO: 90 - protein, 224 aa, synthetic construct
  REGION 1..224: note = caninized mouse antibody
DIVMTQTPLSLSVSPGEPASMSCKSSQSLLNSVNQKNYLAWYRQKPGQSPQVLVYFASTRVSGVPDRFIGSGSGTDFTLRISRVEADDLGVYYCQQYFSTPLTFGQGTKLELKRNDAQPAVYLFQPSPDQLHTGSASVVCLLNSFYPKDINVKWKVDGVIQDTGIQESVTEQDSKDSTYSLSSTLTMSSTEYLSHELYSCEITHKSLPSTLIKSFQRSECQRVD

SEQ ID NO: 91 - DNA, 1338 bp, synthetic construct
  misc_feature 1..1338: note = caninized mouse antibody
  source 1..1338: mol_type = other DNA
gaggtgcagctggtgcagagcggcgccgaggtgaagaagcccggcgccagcgtgaaggtgagctgcaaggccagcggctacaccttcaccacctacggcatgagctgggtgagacaggcccccggcaagggcctgcagtggatgggctggatcaacatctacagcggcatccccacctacgccgacgacttcaagggcagattcgccctgagcctggacaccagcaccagcaccgcctacatggagctgaacagcctgagagccgaggacaccgccgtgtactactgcgccagattcgacggccccgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgcacagcctgagcagcatggtgaccgtgcccagcagcagatggcccagcgagaccttcacctgcaacgtggtgcaccccgccagcaacaccaaggtggacaagcccgtgttcaacgagtgcagatgcaccgacacccccccctgccccgtgcccgagcccctgggcggccccagcgtgctgatcttcccccccaagcccaaggacatcctgagaatcaccagaacccccgaggtgacctgcgtggtgctggacctgggcagagaggaccccgaggtgcagatcagctggttcgtggacggcaaggaggtgcacaccgccaagacccagagcagagagcagcagttcaacggcacctacagagtggtgagcgtgctgcccatcgagcaccaggactggctgaccggcaaggagttcaagtgcagagtgaaccacatcgacctgcccagccccatcgagagaaccatcagcaaggccagaggcagagcccacaagcccagcgtgtacgtgctgccccccagccccaaggagctgagcagcagcgacaccgtgagcatcacctgcctgatcaaggacttctacccccccgacatcgacgtggagtggcagagcaacggccagcaggagcccgagagaaagcacagaatgacccccccccagctggacgaggacggcagctacttcctgtacagcaagctgagcgtggacaagagcagatggcagcagggcgaccccttcacctgcgccgtgatgcacgagaccctgcagaaccactacaccgacctgagcctgagccacagccccggcaag

SEQ ID NO: 92 - protein, 446 aa, synthetic construct
  REGION 1..446: note = caninized mouse antibody
EVQLVQSGAEVKKPGASVKVSCKASGYTFTTYGMSWVRQAPGKGLQWMGWINIYSGIPTYADDFKGRFALSLDTSTSTAYMELNSLRAEDTAVYYCARFDGPDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 93 - DNA, 1338 bp, synthetic construct
  misc_feature 1..1338: note = caninized mouse antibody
  source 1..1338: mol_type = other DNA
gaggtgcagctggtgcagagcggcgccgaggtgaagaagcccggcgccagcgtgaaggtgagctgcaaggccagcggctacaccttcaccacctacggcatgagctgggtgagacaggcccccggcaagggcctgcagtggatgggctggatcaacatctacagcggcatccccacctacgccgacgacttcaagggcagattcgccctgagcctggacaccagcaccagcaccgcctacatggagctgaacagcctgagagccgaggacaccgccgtgtactactgcgccagattcgacggccccgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcaccgtgaccgtgcccagcagcagatggcccagcgagaccttcacctgcaacgtggtgcaccccgccagcaacaccaaggtggacaagcccgtgcccaaggagagcacctgcaagtgcatcagcccctgccccgtgcccgagagcctgggcggccccagcgtgttcatcttcccccccaagcccaaggacatcctgagaatcaccagaacccccgagatcacctgcgtggtgctggacctgggcagagaggaccccgaggtgcagatcagctggttcgtggacggcaaggaggtgcacaccgccaagacccagcccagagagcagcagttcaacagcacctacagagtggtgagcgtgctgcccatcgagcaccaggactggctgaccggcaaggagttcaagtgcagagtgaaccacatcggcctgcccagccccatcgagagaaccatcagcaaggccagaggccaggcccaccagcccagcgtgtacgtgctgccccccagccccaaggagctgagcagcagcgacaccgtgaccctgacctgcctgatcaaggacttcttcccccccgagatcgacgtggagtggcagagcaacggccagcccgagcccgagagcaagtaccacaccaccgccccccagctggacgaggacggcagctacttcctgtacagcaagctgagcgtggacaagagcagatggcagcagggcgacaccttcacctgcgccgtgatgcacgaggccctgcagaaccactacaccgacctgagcctgagccacagccccggcaag

SEQ ID NO: 94 - protein, 446 aa, synthetic construct
  REGION 1..446: note = caninized mouse antibody
EVQLVQSGAEVKKPGASVKVSCKASGYTFTTYGMSWVRQAPGKGLQWMGWINIYSGIPTYADDFKGRFALSLDTSTSTAYMELNSLRAEDTAVYYCARFDGPDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 95 - DNA, 672 bp, synthetic construct; identical to SEQ ID NO: 89
  misc_feature 1..672: note = caninized mouse antibody
  source 1..672: mol_type = other DNA

SEQ ID NO: 96 - protein, 224 aa, synthetic construct; identical to SEQ ID NO: 90
  REGION 1..224: note = caninized mouse antibody

SEQ ID NO: 97 - DNA, 1353 bp, synthetic construct
  misc_feature 1..1353: note = caninized mouse antibody
  source 1..1353: mol_type = other DNA
gaggtgcagctggtgcagagcgtggccgagctggtgaagcccggcgccagcgtgaaggtgagctgcaccgtgagcggcttcaacatcaagaacacctacatgcactgggtgagacaggcccccggcaagggcctgcagtggatcggcagaatcgaccccgccaacgtgaacaccaagtacgcccccaagttccagggcagagccaccatcaccgccgacaccagcaccaacaccgcctacatgcagctgagcagcctgagagccgaggacaccgccgtgtactactgcgccagaatcttctacgactacgacggcgacatcgacgtgtggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgcacagcctgagcagcatggtgaccgtgcccagcagcagatggcccagcgagaccttcacctgcaacgtggtgcaccccgccagcaacaccaaggtggacaagcccgtgttcaacgagtgcagatgcaccgacacccccccctgccccgtgcccgagcccctgggcggccccagcgtgctgatcttcccccccaagcccaaggacatcctgagaatcaccagaacccccgaggtgacctgcgtggtgctggacctgggcagagaggaccccgaggtgcagatcagctggttcgtggacggcaaggaggtgcacaccgccaagacccagagcagagagcagcagttcaacggcacctacagagtggtgagcgtgctgcccatcgagcaccaggactggctgaccggcaaggagttcaagtgcagagtgaaccacatcgacctgcccagccccatcgagagaaccatcagcaaggccagaggcagagcccacaagcccagcgtgtacgtgctgccccccagccccaaggagctgagcagcagcgacaccgtgagcatcacctgcctgatcaaggacttctacccccccgacatcgacgtggagtggcagagcaacggccagcaggagcccgagagaaagcacagaatgacccccccccagctggacgaggacggcagctacttcctgtacagcaagctgagcgtggacaagagcagatggcagcagggcgaccccttcacctgcgccgtgatgcacgagaccctgcagaaccactacaccgacctgagcctgagccacagccccggcaag

SEQ ID NO: 98 - protein, 451 aa, synthetic construct
  REGION 1..451: note = caninized mouse antibody
EVQLVQSVAELVKPGASVKVSCTVSGFNIKNTYMHWVRQAPGKGLQWIGRIDPANVNTKYAPKFQGRATITADTSTNTAYMQLSSLRAEDTAVYYCARIFYDYDGDIDVWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 99 - DNA, 1353 bp, synthetic construct
  misc_feature 1..1353: note = caninized mouse antibody
  source 1..1353: mol_type = other DNA
gaggtgcagctggtgcagagcgtggccgagctggtgaagcccggcgccagcgtgaaggtgagctgcaccgtgagcggcttcaacatcaagaacacctacatgcactgggtgagacaggcccccggcaagggcctgcagtggatcggcagaatcgaccccgccaacgtgaacaccaagtacgcccccaagttccagggcagagccaccatcaccgccgacaccagcaccaacaccgcctacatgcagctgagcagcctgagagccgaggacaccgccgtgtactactgcgccagaatcttctacgactacgacggcgacatcgacgtgtggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcaccgtgaccgtgcccagcagcagatggcccagcgagaccttcacctgcaacgtggtgcaccccgccagcaacaccaaggtggacaagcccgtgcccaaggagagcacctgcaagtgcatcagcccctgccccgtgcccgagagcctgggcggccccagcgtgttcatcttcccccccaagcccaaggacatcctgagaatcaccagaacccccgagatcacctgcgtggtgctggacctgggcagagaggaccccgaggtgcagatcagctggttcgtggacggcaaggaggtgcacaccgccaagacccagcccagagagcagcagttcaacagcacctacagagtggtgagcgtgctgcccatcgagcaccaggactggctgaccggcaaggagttcaagtgcagagtgaaccacatcggcctgcccagccccatcgagagaaccatcagcaaggccagaggccaggcccaccagcccagcgtgtacgtgctgccccccagccccaaggagctgagcagcagcgacaccgtgaccctgacctgcctgatcaaggacttcttcccccccgagatcgacgtggagtggcagagcaacggccagcccgagcccgagagcaagtaccacaccaccgccccccagctggacgaggacggcagctacttcctgtacagcaagctgagcgtggacaagagcagatggcagcagggcgacaccttcacctgcgccgtgatgcacgaggccctgcagaaccactacaccgacctgagcctgagccacagccccggcaag

SEQ ID NO: 100 - protein, 451 aa, synthetic construct
  REGION 1..451: note = caninized mouse antibody
EVQLVQSVAELVKPGASVKVSCTVSGFNIKNTYMHWVRQAPGKGLQWIGRIDPANVNTKYAPKFQGRATITADTSTNTAYMQLSSLRAEDTAVYYCARIFYDYDGDIDVWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 101 - DNA, 654 bp, synthetic construct
  misc_feature 1..654: note = caninized mouse antibody
  source 1..654: mol_type = other DNA
gacatcgtgatgacccagacccccctgagcctgagcgtgagcctgggcgagcccgccagcatcagctgccacgccagccagaacatcaacgtgtggctgagctggtacagacagaagcccggccagatcccccagctgctgatctacaaggccagcaacctgcacaccggcgtgcccgacagattcagcggcagcggcagcggcaccgacttcaccctgagaatcagcagagtggaggccgacgacgccggcgtgtactactgccagcagggccagagctaccccctgaccttcggccagggcaccaaggtggagatcaagagaaacgacgcccagcccgccgtgtacctgttccagcccagccccgaccagctgcacaccggcagcgccagcgtggtgtgcctgctgaacagcttctaccccaaggacatcaacgtgaagtggaaggtggacggcgtgatccaggacaccggcatccaggagagcgtgaccgagcaggacagcaaggacagcacctacagcctgagcagcaccctgaccatgagcagcaccgagtacctgagccacgagctgtacagctgcgagatcacccacaagagcctgcccagcaccctgatcaagagcttccagagaagcgagtgccagagagtggac

SEQ ID NO: 102 - protein, 218 aa, synthetic construct
  REGION 1..218: note = caninized mouse antibody
DIVMTQTPLSLSVSLGEPASISCHASQNINVWLSWYRQKPGQIPQLLIYKASNLHTGVPDRFSGSGSGTDFTLRISRVEADDAGVYYCQQGQSYPLTFGQGTKVEIKRNDAQPAVYLFQPSPDQLHTGSASVVCLLNSFYPKDINVKWKVDGVIQDTGIQESVTEQDSKDSTYSLSSTLTMSSTEYLSHELYSCEITHKSLPSTLIKSFQRSECQRVD

SEQ ID NO: 103 - DNA, 1338 bp, synthetic construct
  misc_feature 1..1338: note = caninized mouse antibody
  source 1..1338: mol_type = other DNA
gaggtgcagctggtgcagagcggcgccgaggtgaagaagcccggcgccagcgtgaaggtgagctgcaaggccagcggctacaccttcaccacctacggcatgagctgggtgagacaggcccccggcaagggcctgcagtggatgggctggatcaacatctacagcggcatgcccacctacgccgacgacttcaagggcagattcgccctgagcctggacaccagcaccagcaccgcctacatggagctgaacagcctgagagccgaggacaccgccgtgtactactgcaccagattcgacggccccgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgcacagcctgagcagcatggtgaccgtgcccagcagcagatggcccagcgagaccttcacctgcaacgtggtgcaccccgccagcaacaccaaggtggacaagcccgtgttcaacgagtgcagatgcaccgacacccccccctgccccgtgcccgagcccctgggcggccccagcgtgctgatcttcccccccaagcccaaggacatcctgagaatcaccagaacccccgaggtgacctgcgtggtgctggacctgggcagagaggaccccgaggtgcagatcagctggttcgtggacggcaaggaggtgcacaccgccaagacccagagcagagagcagcagttcaacggcacctacagagtggtgagcgtgctgcccatcgagcaccaggactggctgaccggcaaggagttcaagtgcagagtgaaccacatcgacctgcccagccccatcgagagaaccatcagcaaggccagaggcagagcccacaagcccagcgtgtacgtgctgccccccagccccaaggagctgagcagcagcgacaccgtgagcatcacctgcctgatcaaggacttctacccccccgacatcgacgtggagtggcagagcaacggccagcaggagcccgagagaaagcacagaatgacccccccccagctggacgaggacggcagctacttcctgtacagcaagctgagcgtggacaagagcagatggcagcagggcgaccccttcacctgcgccgtgatgcacgagaccctgcagaaccactacaccgacctgagcctgagccacagccccggcaag

SEQ ID NO: 104 - protein, 446 aa, synthetic construct
  REGION 1..446: note = caninized mouse antibody
EVQLVQSGAEVKKPGASVKVSCKASGYTFTTYGMSWVRQAPGKGLQWMGWINIYSGMPTYADDFKGRFALSLDTSTSTAYMELNSLRAEDTAVYYCTRFDGPDYWGQGTLVTVSSASTTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 105 - DNA, 1338 bp, synthetic construct
  misc_feature 1..1338: note = caninized mouse antibody
  source 1..1338: mol_type = other DNA
gaggtgcagctggtgcagagcggcgccgaggtgaagaagcccggcgccagcgtgaaggtgagctgcaaggccagcggctacaccttcaccacctacggcatgagctgggtgagacaggcccccggcaagggcctgcagtggatgggctggatcaacatctacagcggcatgcccacctacgccgacgacttcaagggcagattcgccctgagcctggacaccagcaccagcaccgcctacatggagctgaacagcctgagagccgaggacaccgccgtgtactactgcaccagattcgacggccccgactactggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcaccgtgaccgtgcccagcagcagatggcccagcgagaccttcacctgcaacgtggtgcaccccgccagcaacaccaaggtggacaagcccgtgcccaaggagagcacctgcaagtgcatcagcccctgccccgtgcccgagagcctgggcggccccagcgtgttcatcttcccccccaagcccaaggacatcctgagaatcaccagaacccccgagatcacctgcgtggtgctggacctgggcagagaggaccccgaggtgcagatcagctggttcgtggacggcaaggaggtgcacaccgccaagacccagcccagagagcagcagttcaacagcacctacagagtggtgagcgtgctgcccatcgagcaccaggactggctgaccggcaaggagttcaagtgcagagtgaaccacatcggcctgcccagccccatcgagagaaccatcagcaaggccagaggccaggcccaccagcccagcgtgtacgtgctgccccccagccccaaggagctgagcagcagcgacaccgtgaccctgacctgcctgatcaaggacttcttcccccccgagatcgacgtggagtggcagagcaacggccagcccgagcccgagagcaagtaccacaccaccgccccccagctggacgaggacggcagctacttcctgtacagcaagctgagcgtggacaagagcagatggcagcagggcgacaccttcacctgcgccgtgatgcacgaggccctgcagaaccactacaccgacctgagcctgagccacagccccggcaag

SEQ ID NO: 106 - protein, 446 aa, synthetic construct
  REGION 1..446: note = caninized mouse antibody
EVQLVQSGAEVKKPGASVKVSCKASGYTFTTYGMSWVRQAPGKGLQWMGWINIYSGMPTYADDFKGRFALSLDTSTSTAYMELNSLRAEDTAVYYCTRFDGPDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 107 - DNA, 672 bp, synthetic construct
  misc_feature 1..672: note = caninized mouse antibody
  source 1..672: mol_type = other DNA
gacatcgtgatgacccagacccccctgagcctgagcgtgagccccggcgagcccgccagcatgagctgcaagagcagccagagcctgctgaacagcgtgaaccagaagaactacctggcctggtacagacagaagcccggccagagcccccaggtgctggtgtacttcgccagcaccagaatcagcggcgtgcccgacagattcatcggcagcggcagcggcaccgacttcaccctgagaatcagcagagtggaggccgacgacctgggcgtgtactactgccagcagtacttcagcacccccctgaccttcggccagggcaccaagctggagctgaagagaaacgacgcccagcccgccgtgtacctgttccagcccagccccgaccagctgcacaccggcagcgccagcgtggtgtgcctgctgaacagcttctaccccaaggacatcaacgtgaagtggaaggtggacggcgtgatccaggacaccggcatccaggagagcgtgaccgagcaggacagcaaggacagcacctacagcctgagcagcaccctgaccatgagcagcaccgagtacctgagccacgagctgtacagctgcgagatcacccacaagagcctgcccagcaccctgatcaagagcttccagagaagcgagtgccagagagtggac

SEQ ID NO: 108 - protein, 224 aa, synthetic construct
  REGION 1..224: note = caninized mouse antibody
DIVMTQTPLSLSVSPGEPASMSCKSSQSLLNSVNQKNYLAWYRQKPGQSPQVLVYFASTRISGVPDRFIGSGSGTDFTLRISRVEADDLGVYYCQQYFSTPLTFGQGTKLELKRNDAQPAVYLFQPSPDQLHTGSASVVCLLNSFYPKDINVKWKVDGVIQDTGIQESVTEQDSKDSTYSLSSTLTMSSTEYLSHELYSCEITHKSLPSTLIKSFQRSECQRVD

SEQ ID NO: 109 - protein, 17 aa, Canis lupus
  source 1..17: note = Canis familiaris
FNECRCTDTPPCPVPEP

SEQ ID NO: 110 - protein, 22 aa, Canis lupus
  source 1..22: note = Canis familiaris
PKRENGRVPRPPDCPKCPAPEM

SEQ ID NO: 111 - protein, 20 aa, Canis lupus
  source 1..20: note = Canis familiaris
AKECECKCNCNNCPCPGCGL

SEQ ID NO: 112 - protein, 17 aa, synthetic construct
  REGION 1..17: note = modified canine
PKESTCKCIPPCPVPES

SEQ ID NO: 113 - DNA, 795 bp, Canis lupus
  source 1..795: mol_type = unassigned DNA; note = Canis familiaris
ctagactcccctgacaggccctggagcccgctcaccttctccccggcgcagctcacggtgcaggagggagagaacgccacgttcacctgcagcctggccgacatccccgacagcttcgtgctcaactggtaccgcctgagcccccgcaaccagacggacaagctggccgccttccaggaggaccgcatcgagccgggccgggacaggcgcttccgcgtcatgcggctgcccaacgggcgggacttccacatgagcatcgtcgctgcgcgcctcaacgacagcggcatctacctgtgcggggccatctacctgccccccaacacacagatcaacgagagtccccgcgcagagctctccgtgacggagagaaccctggagccccccacacagagccccagccccccacccagactcagcggccagttgcaggggctggtcatcggcgtcacgagcgtgctggtgggtgtcctgctactgctgctgctgacctgggtcctggccgctgtcttccccagggccacccgaggtgcctgtgtgtgcgggagcgaggacgagcctctgaaggagggccccgatgcagcgcccgtcttcaccctggactacggggagctggacttccagtggcgagagaagacgccggagcccccggcgccctgtgccccggagcagaccgagtatgccaccatcgtcttcccgggcaggccggcgtccccgggccgcagggcctcggccagcagcctgcagggagcccagcctccgagccccgaggacggacccggcctgtggcccctctga

SEQ ID NO: 114 - protein, 264 aa, Canis lupus
  source 1..264: note = Canis familiaris
LDSPDRPWSPLTFSPAQLTVQEGENATFTCSLADIPDSFVLNWYRLSPRNQTDKLAAFQEDRIEPGRDRRFRVMRLPNGRDFHMSIVAARLNDSGIYLCGAIYLPPNTQINESPRAELSVTERTLEPPTQSPSPPPRLSGQLQGLVIGVTSVLVGVLLLLLLTWVLAAVFPRATRGACVCGSEDEPLKEGPDAAPVFTLDYGELDFQWREKTPEPPAPCAPEQTEYATIVFPGRPASPGRRASASSLQGAQPPSPEDGPGLWPL

SEQ ID NO: 115 - DNA, 438 bp, Canis lupus
  source 1..438: mol_type = unassigned DNA; note = Canis familiaris
ctggattcccccgacagaccctggagccctctcaccttctcccctgcccagctgaccgtccaggaaggcgagaatgccaccttcacctgcagcctcgccgacatccccgacagcttcgtgctgaactggtacagactgagccccaggaaccagaccgacaagctggccgctttccaggaggacaggatcgaacccggcagggacaggaggtttagggtcatgaggctgcccaacggcagggacttccacatgtccatcgtggccgccagactgaacgactccggcatctacctgtgcggcgctatctacctgccccccaacacccagatcaacgagagccccagggccgaactgagcgtgacagagagaaccctggaacctcccacccagagcccttcccctcctcctagactgagcggacagctgcagggcctggtg

SEQ ID NO: 116 - protein, 146 aa, Canis lupus
  source 1..146: note = Canis familiaris
LDSPDRPWSPLTFSPAQLTVQEGENATFTCSLADIPDSFVLNWYRLSPRNQTDKLAAFQEDRIEPGRDRRFRVMRLPNGRDFHMSIVAARLNDSGIYLCGAIYLPPNTQINESPRAELSVTERTLEPPTQSPSPPPRLSGQLQGLV

SEQ ID NO: 117 - DNA, 1128 bp, synthetic construct
  misc_feature 1..1128: note = canine and human sequence
  source 1..1128: mol_type = other DNA
ctggattcccccgacagaccctggagccctctcaccttctcccctgcccagctgaccgtccaggaaggcgagaatgccaccttcacctgcagcctcgccgacatccccgacagcttcgtgctgaactggtacagactgagccccaggaaccagaccgacaagctggccgctttccaggaggacaggatcgaacccggcagggacaggaggtttagggtcatgaggctgcccaacggcagggacttccacatgtccatcgtggccgccagactgaacgactccggcatctacctgtgcggcgctatctacctgccccccaacacccagatcaacgagagccccagggccgaactgagcgtgacagagagaaccctggaacctcccacccagagcccttcccctcctcctagactgagcggacagctgcagggcctggtgggtaccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 118 - protein, 375 aa, synthetic construct
  REGION 1..375: note = canine and human sequence
LDSPDRPWSPLTFSPAQLTVQEGENATFTCSLADIPDSFVLNWYRLSPRNQTDKLAAFQEDRIEPGRDRRFRVMRLPNGRDFHMSIVAARLNDSGIYLCGAIYLPPNTQINESPRAELSVTERTLEPPTQSPSPPPRLSGQLQGLVGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - DNA, 816 bp, Canis lupus
  source 1..816: mol_type = unassigned DNA; note = Canis familiaris
tttacgatcacagtttctaaggacctgtatgtggtagagtatggtggcaatgtgacaatggaatgcaaattcccggtggaaaaacagttaaacttgtttgcactaatcgtctactgggaaatggaggataaaaaaattatacaatttgtgaatggaaaggaagacctgaaagttcagcacagcagctacagccagagggctcagctattgaaggaccagctcttcttggggaaggctgcgcttcagatcacagatgtgagattgcaggatgcaggggtttactgctgcttgatcggctatggcggtgctgactacaagcggattactttgaaagttcatgccccgtaccgcaacatcagccaaagaatttctgtggatcctgtcacctctgaacatgaactaatgtgtcaggctgagggttaccctgaggctgaagtcatctggacaagcagtgaccaccgagtcctgagtggcaaaaccaccatcactaattccaatagggaagagaagcttttcaatgtgaccagcacgctgaacatcaatgcaacagctaatgagattttctactgcacttttcaaagatcaggtcctgaggaaaacaatactgccgagttggtcatcccagaacgactgcccgttccagcaagtgagaggactcatttcatgattctgggacctttcctgttgcttcttggtgtagtcctggcagtcactttctgtctaaaaaaacatgggagaatgatggatgtggaaaaatgttgcacccgagataggaactcaaagaaacgaaatgatatacaatttgaagagacataa

SEQ ID NO: 120 - protein, 271 aa, Canis lupus
  source 1..271: note = Canis familiaris
FTITVSKDLYVVEYGGNVTMECKFPVEKQLNLFALIVYWEMEDKKIIQFVNGKEDLKVQHSSYSQRAQLLKDQLFLGKAALQITDVRLQDAGVYCCLIGYGGADYKRITLKVHAPYRNISQRISVDPVTSEHELMCQAEGYPEAEVIWTSSDHRVLSGKTTITNSNREEKLFNVTSTLNINATANEIFYCTFQRSGPEENNTAELVIPERLPVPASERTHFMILGPFLLLLGVVLAVTFCLKKHGRMMDVEKCCTRDRNSKKRNDIQFEET

SEQ ID NO: 121 - DNA, 660 bp, Canis lupus
  source 1..660: mol_type = unassigned DNA; note = Canis familiaris
tttaccatcaccgtgtccaaggacctgtacgtggtcgagtacggcggcaatgtgaccatggagtgcaagttccccgtggagaagcagctgaacctgttcgccctcatcgtgtactgggagatggaggacaagaagatcatccagttcgtgaacggcaaggaggacctgaaggtgcagcactccagctactcccagagagcccagctgctgaaggaccagctgttcctgggcaaggccgccctgcagatcaccgacgtgagactgcaggacgccggcgtgtattgctgcctgatcggctacggaggcgccgactacaagaggatcaccctgaaggtgcatgcaccctacaggaacatcagccagaggatcagcgtcgatcccgtgaccagcgagcacgagctgatgtgccaagccgagggctatcccgaggccgaagtgatctggaccagcagcgaccacagggtcctgagcggcaagaccaccatcaccaacagcaacagggaggagaagctgttcaacgtgaccagcaccctcaacatcaacgccaccgccaacgagatcttctactgcaccttccagaggagcggccccgaagagaacaacaccgccgagctggtgatccccgagagactgcctgtgcctgccagcgagaggacccac

SEQ ID NO: 122 - protein, 220 aa, Canis lupus
  source 1..220: note = Canis familiaris
FTITVSKDLYVVEYGGNVTMECKFPVEKQLNLFALIVYWEMEDKKIIQFVNGKEDLKVQHSSYSQRAQLLKDQLFLGKAALQITDVRLQDAGVYCCLIGYGGADYKRITLKVHAPYRNISQRISVDPVTSEHELMCQAEGYPEAEVIWTSSDHRVLSGKTTITNSNREEKLFNVTSTLNINATANEIFYCTFQRSGPEENNTAELVIPERLPVPASERTH

SEQ ID NO: 123 - DNA, 1350 bp, Canis lupus
  source 1..1350: mol_type = unassigned DNA; note = Canis familiaris
tttaccatcaccgtgtccaaggacctgtacgtggtcgagtacggcggcaatgtgaccatggagtgcaagttccccgtggagaagcagctgaacctgttcgccctcatcgtgtactgggagatggaggacaagaagatcatccagttcgtgaacggcaaggaggacctgaaggtgcagcactccagctactcccagagagcccagctgctgaaggaccagctgttcctgggcaaggccgccctgcagatcaccgacgtgagactgcaggacgccggcgtgtattgctgcctgatcggctacggaggcgccgactacaagaggatcaccctgaaggtgcatgcaccctacaggaacatcagccagaggatcagcgtcgatcccgtgaccagcgagcacgagctgatgtgccaagccgagggctatcccgaggccgaagtgatctggaccagcagcgaccacagggtcctgagcggcaagaccaccatcaccaacagcaacagggaggagaagctgttcaacgtgaccagcaccctcaacatcaacgccaccgccaacgagatcttctactgcaccttccagaggagcggccccgaagagaacaacaccgccgagctggtgatccccgagagactgcctgtgcctgccagcgagaggacccacggtaccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 124 - protein, 449 aa, Canis lupus
  source 1..449: note = Canis familiaris
FTITVSKDLYVVEYGGNVTMECKFPVEKQLNLFALIVYWEMEDKKIIQFVNGKEDLKVQHSSYSQRAQLLKDQLFLGKAALQITDVRLQDAGVYCCLIGYGGADYKRITLKVHAPYRNISQRISVDPVTSEHELMCQAEGYPEAEVIWTSSDHRVLSGKTTITNSNREEKLFNVTSTLNINATANEIFYCTFQRSGPEENNTAELVIPERLPVPASERTHGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - DNA, 648 bp, Canis lupus
  source 1..648: mol_type = unassigned DNA; note = Canis familiaris
ctgggcggccccagcgtgctgatcttcccccccaagcccaaggacatcctgagaatcaccagaacccccgaggtgacctgcgtggtgctggacctgggcagagaggaccccgaggtgcagatcagctggttcgtggacggcaaggaggtgcacaccgccaagacccagagcagagagcagcagttcaacggcacctacagagtggtgagcgtgctgcccatcgagcaccaggactggctgaccggcaaggagttcaagtgcagagtgaaccacatcgacctgcccagccccatcgagagaaccatcagcaaggccagaggcagagcccacaagcccagcgtgtacgtgctgccccccagccccaaggagctgagcagcagcgacaccgtgagcatcacctgcctgatcaaggacttctacccccccgacatcgacgtggagtggcagagcaacggccagcaggagcccgagagaaagcacagaatgacccccccccagctggacgaggacggcagctacttcctgtacagcaagctgagcgtggacaagagcagatggcagcagggcgaccccttcacctgcgccgtgatgcacgagaccctgcagaaccactacaccgacctgagcctgagccacagccccggcaag

SEQ ID NO: 126 - protein, 216 aa, Canis lupus
  source 1..216: note = Canis familiaris
LGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 127 - DNA, 648 bp, Canis lupus
  source 1..648: mol_type = unassigned DNA; note = Canis familiaris
ctgggcggccccagcgtgttcatcttcccccccaagcccaaggacatcctgagaatcaccagaacccccgagatcacctgcgtggtgctggacctgggcagagaggaccccgaggtgcagatcagctggttcgtggacggcaaggaggtgcacaccgccaagacccagcccagagagcagcagttcaacagcacctacagagtggtgagcgtgctgcccatcgagcaccaggactggctgaccggcaaggagttcaagtgcagagtgaaccacatcggcctgcccagccccatcgagagaaccatcagcaaggccagaggccaggcccaccagcccagcgtgtacgtgctgccccccagccccaaggagctgagcagcagcgacaccgtgaccctgacctgcctgatcaaggacttcttcccccccgagatcgacgtggagtggcagagcaacggccagcccgagcccgagagcaagtaccacaccaccgccccccagctggacgaggacggcagctacttcctgtacagcaagctgagcgtggacaagagcagatggcagcagggcgacaccttcacctgcgccgtgatgcacgaggccctgcagaaccactacaccgacctgagcctgagccacagccccggcaag

SEQ ID NO: 128 - protein, 216 aa, Canis lupus
  source 1..216: note = Canis familiaris
LGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGK

SEQ ID NO: 129 - DNA, 645 bp, Canis lupus
  source 1..645: mol_type = unassigned DNA; note = Canis familiaris
ctgggcggccccagcgtgttcatcttcccccccaagcccaaggacaccctgctgatcgccagaacccccgaggtgacctgcgtggtggtggacctggaccccgaggaccccgaggtgcagatcagctggttcgtggacggcaagcagatgcagaccgccaagacccagcccagagaggagcagttcaacggcacctacagagtggtgagcgtgctgcccatcggccaccaggactggctgaagggcaagcagttcacctgcaaggtgaacaacaaggccctgcccagccccatcgagagaaccatcagcaaggccagaggccaggcccaccagcccagcgtgtacgtgctgccccccagcagagaggagctgagcaagaacaccgtgagcctgacctgcctgatcaaggacttcttcccccccgacatcgacgtggagtggcagagcaacggccagcaggagcccgagagcaagtacagaaccacccccccccagctggacgaggacggcagctacttcctgtacagcaagctgagcgtggacaagagcagatggcagagaggcgacaccttcatctgcgccgtgatgcacgaggccctgcacaaccactacacccaggagagcctgagccacagccccggcaag

SEQ ID NO: 130 - protein, 215 aa, Canis lupus
  source 1..215: note = Canis familiaris
LGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 131 - DNA, 645 bp, Canis lupus
  source 1..645: mol_type = unassigned DNA; note = Canis familiaris
ctgggcggccccagcgtgttcatcttcccccccaagcccaaggacatcctggtgaccgccagaacccccaccgtgacctgcgtggtggtggacctggaccccgagaaccccgaggtgcagatcagctggttcgtggacagcaagcaggtgcagaccgccaacacccagcccagagaggagcagagcaacggcacctacagagtggtgagcgtgctgcccatcggccaccaggactggctgagcggcaagcagttcaagtgcaaggtgaacaacaaggccctgcccagccccatcgaggagatcatcagcaagacccccggccaggcccaccagcccaacgtgtacgtgctgccccccagcagagacgagatgagcaagaacaccgtgaccctgacctgcctggtgaaggacttcttcccccccgagatcgacgtggagtggcagagcaacggccagcaggagcccgagagcaagtacagaatgacccccccccagctggacgaggacggcagctacttcctgtacagcaagctgagcgtggacaagagcagatggcagagaggcgacaccttcatctgcgccgtgatgcacgaggccctgcacaaccactacacccagatcagcctgagccacagccccggcaag

SEQ ID NO: 132 - protein, 215 aa, Canis lupus
  source 1..215: note = Canis familiaris
LGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 133 - DNA, 867 bp, Canis lupus
  source 1..867: mol_type = unassigned DNA; note = Canis familiaris
atggggagccggcgggggccctggccgctcgtctgggccgtgctgcagctgggctggtggccaggatggctcctagactcccctgacaggccctggagcccgctcaccttctccccggcgcagctcacggtgcaggagggagagaacgccacgttcacctgcagcctggccgacatccccgacagcttcgtgctcaactggtaccgcctgagcccccgcaaccagacggacaagctggccgccttccaggaggaccgcatcgagccgggccgggacaggcgcttccgcgtcatgcggctgcccaacgggcgggacttccacatgagcatcgtcgctgcgcgcctcaacgacagcggcatctacctgtgcggggccatctacctgccccccaacacacagatcaacgagagtccccgcgcagagctctccgtgacggagagaaccctggagccccccacacagagccccagccccccacccagactcagcggccagttgcaggggctggtcatcggcgtcacgagcgtgctggtgggtgtcctgctactgctgctgctgacctgggtcctggccgctgtcttccccagggccacccgaggtgcctgtgtgtgcgggagcgaggacgagcctctgaaggagggccccgatgcagcgcccgtcttcaccctggactacggggagctggacttccagtggcgagagaagacgccggagcccccggcgccctgtgccccggagcagaccgagtatgccaccatcgtcttcccgggcaggccggcgtccccgggccgcagggcctcggccagcagcctgcagggagcccagcctccgagccccgaggacggacccggcctgtggcccctctga

SEQ ID NO: 134 - protein, 288 aa, Canis lupus
  source 1..288: note = Canis familiaris
MGSRRGPWPLVWAVLQLGWWPGWLLDSPDRPWSPLTFSPAQLTVQEGENATFTCSLADIPDSFVLNWYRLSPRNQTDKLAAFQEDRIEPGRDRRFRVMRLPNGRDFHMSIVAARLNDSGIYLCGAIYLPPNTQINESPRAELSVTERTLEPPTQSPSPPPRLSGQLQGLVIGVTSVLVGVLLLLLLTWVLAAVFPRATRGACVCGSEDEPLKEGPDAAPVFTLDYGELDFQWREKTPEPPAPCAPEQTEYATIVFPGRPASPGRRASASSLQGAQPPSPEDGPGLWPL

SEQ ID NO: 135 - DNA, 870 bp, Canis lupus
  source 1..870: mol_type = unassigned DNA; note = Canis familiaris
atgagaatgtttagtgtctttacattcatggcctactgccatttgctaaaagcatttacgatcacagtttctaaggacctgtatgtggtagagtatggtggcaatgtgacaatggaatgcaaattcccggtggaaaaacagttaaacttgtttgcactaatcgtctactgggaaatggaggataaaaaaattatacaatttgtgaatggaaaggaagacctgaaagttcagcacagcagctacagccagagggctcagctattgaaggaccagctcttcttggggaaggctgcgcttcagatcacagatgtgagattgcaggatgcaggggtttactgctgcttgatcggctatggcggtgctgactacaagcggattactttgaaagttcatgccccgtaccgcaacatcagccaaagaatttctgtggatcctgtcacctctgaacatgaactaatgtgtcaggctgagggttaccctgaggctgaagtcatctggacaagcagtgaccaccgagtcctgagtggcaaaaccaccatcactaattccaatagggaagagaagcttttcaatgtgaccagcacgctgaacatcaatgcaacagctaatgagattttctactgcacttttcaaagatcaggtcctgaggaaaacaatactgccgagttggtcatcccagaacgactgcccgttccagcaagtgagaggactcatttcatgattctgggacctttcctgttgcttcttggtgtagtcctggcagtcactttctgtctaaaaaaacatgggagaatgatggatgtggaaaaatgttgcacccgagataggaactcaaagaaacgaaatgatatacaatttgaagagacataa

SEQ ID NO: 136 - protein, 289 aa, Canis lupus
  source 1..289: note = Canis familiaris
MRMFSVFTFMAYCHLLKAFTITVSKDLYVVEYGGNVTMECKFPVEKQLNLFALIVYWEMEDKKIIQFVNGKEDLKVQHSSYSQRAQLLKDQLFLGKAALQITDVRLQDAGVYCCLIGYGGADYKRITLKVHAPYRNISQRISVDPVTSEHELMCQAEGYPEAEVIWTSSDHRVLSGKTTITNSNREEKLFNVTSTLNINATANEIFYCTFQRSGPEENNTAELVIPERLPVPASERTHFMILGPFLLLLGVVLAVTFCLKKHGRMMDVEKCCTRDRNSKKRNDIQFEET

SEQ ID NO: 137 - protein, 401 aa, Canis lupus
  source 1..401: note = Canis familiaris
MNFLLSWVHWSLALLLYLHHAKWSQALDSPDRPWSPLTFSPAQLTVQEGENATFTCSLADIPDSFVLNWYRLSPRNQTDKLAAFQEDRIEPGRDRRFRVMRLPNGRDFHMSIVAARLNDSGIYLCGAIYLPPNTQINESPRAELSVTERTLEPPTQSPSPPPRLSGQLQGLVGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 15 aa, Canis lupus
  VARIANT 9: note = misc_feature - Xaa can be Met or Thr
  source 1..15: note = Canis familiaris
GRDRRFRVXRLPNGR

SEQ ID NO: 139 - protein, 15 aa, Canis lupus
  VARIANT 14: note = MISC_FEATURE - Xaa can be Met or Thr
  source 1..15: note = Canis familiaris
DRIEPGRDRRFRVXR

SEQ ID NO: 140 - protein, 25 aa, Canis lupus
  VARIANT 5: note = MISC_FEATURE - Xaa can be Met or Thr
  source 1..25: note = Canis familiaris
RFRVXRLPNGRDFHMSIVAARLNDS

SEQ ID NO: 141 - protein, 26 aa, Canis lupus
  VARIANT 20: note = MISC_FEATURE - Xaa can be Met or Thr
  source 1..26: note = Canis familiaris
LAAFQEDRIEPGRDRRFRVXRLPNGR

SEQ ID NO: 142 - protein, 31 aa, Canis lupus
  VARIANT 15: note = MISC_FEATURE - Xaa can be Met or Thr
  source 1..31: note = Canis familiaris
EDRIEPGRDRRFRVXRLPNGRDFHMSIVAAR

SEQ ID NO: 143 - protein, 31 aa, Canis lupus
  VARIANT 25: note = MISC_FEATURE - Xaa can be Met or Thr
  source 1..31: note = Canis familiaris
NQTDKLAAFQEDRIEPGRDRRFRVXRLPNGR

SEQ ID NO: 144 - protein, 45 aa, Canis lupus
  VARIANT 25: note = MISC_FEATURE - Xaa can be Met or Thr
  source 1..45: note = Canis familiaris
NQTDKLAAFQEDRIEPGRDRRFRVXRLPNGRDFHMSIVAARLNDS

SEQ ID NO: 145 - protein, 20 aa, Canis lupus
  VARIANT 14: note = MISC_FEATURE - Xaa can be Met or Thr
  source 1..20: note = Canis familiaris
DRIEPGRDRRFRVXRLPNGR

SEQ ID NO: 146 - protein, 11 aa, Mus musculus
IFYDYDGDIDV